SEQUENCE LISTING (31 sequences)

SEQ ID NO: 1 - DNA, 1323 bp, Triticum aestivum
  variation (140)..(140): T=G in Robigus
  variation (1150)..(1150): G=A in Robigus
atgaacctactgcctcaccaccacctgtcgctgccgtctgggcctggccgccgcccctcctctgcggcggaggcggtggagatggacccgcgcgtgtggcgccgcctgccgcagccgctgctggaccgcgtgctggcgttcctcccgacgccgtccttcctccgcgcccgcgccgtctgccgccgcttctaccacctcctcttctcctccccgttcctccactctcacctcctccactccccgcacctccccttcttcgccttcgccgtcccctccgccggccacctcctcctcctcgatcccacctcccagccgcagggaccctcctggttcctcctcccgctcccgatcccaggtcccgccgcggggttctcgccggctcccgcgtccgctggcctgctggcgttcctctccgacgcgtccggccacaagacgctgctcctcgccaaccccatcacgcgcctcctcgccgcgctgccgctcggccccacgcagcgcctctcccccaccgtcggcctggccgcggggtcgacgtccatcatcgccgtcgtggctggcgacgacctcgtgtcccctttcgccgtcaagaacatctccgtcgacaccttcgtcgccgacgccgcctccgtcccgtcctccggcttctgggcccccagctccctcctgccacgcctgtcctccctcgatcctcgcgccggcatggccttcgcctccggaaggttctactgcatgagctcgtcgccgttcgcggttctcgtgttcgacgtggcggcgaacgtctggagcaaggtgcagccgccgatgaggcggttcctgcagtcgccggcgctggtcgagctcggcggcggcagggagggctcgggcaccgcaagggtggggctcgtcgcgtccgtggagaagagccgtctcagcgtgccgcggagcgtgcgcgtctggacactgcgcggcagaggaggctccggcggcggcggcggcgcgtggagcgaggtggcgcggatgccgcaggacgtgcacgcgcagttcgcggcggcggagggcggccgcgggttcgagtgcgcagcgcacggcgacttcgtcgcgctagcgccccgcggcgggccggcagccgtgccggtgccgacgaccgtgctcgtgttcgactcgcgccgcgacgagtggcggtgggcgccaccatgcccatacgtcgggcacggcatggccgcagtggtcaacggcggaggcgcggggttccgggtcctcgcgtacgagccacgcctggcgacgccggccatcggccttctggacgccacgacgccggtggctttgcatgggatgcatggttag

SEQ ID NO: 2 - DNA, 1457 bp, Triticum aestivum
  exon (1)..(715): exon 1
  variation (140)..(140): T=G in Robigus
  exon (850)..(1457): exon 2
  variation (1284)..(1284): G=A in Robigus
atgaacctactgcctcaccaccacctgtcgctgccgtctgggcctggccgccgcccctcctctgcggcggaggcggtggagatggacccgcgcgtgtggcgccgcctgccgcagccgctgctggaccgcgtgctggcgttcctcccgacgccgtccttcctccgcgcccgcgccgtctgccgccgcttctaccacctcctcttctcctccccgttcctccactctcacctcctccactccccgcacctccccttcttcgccttcgccgtcccctccgccggccacctcctcctcctcgatcccacctcccagccgcagggaccctcctggttcctcctcccgctcccgatcccaggtcccgccgcggggttctcgccggctcccgcgtccgctggcctgctggcgttcctctccgacgcgtccggccacaagacgctgctcctcgccaaccccatcacgcgcctcctcgccgcgctgccgctcggccccacgcagcgcctctcccccaccgtcggcctggccgcggggtcgacgtccatcatcgccgtcgtggctggcgacgacctcgtgtcccctttcgccgtcaagaacatctccgtcgacaccttcgtcgccgacgccgcctccgtcccgtcctccggcttctgggcccccagctccctcctgccacgcctgtcctccctcgatcctcgcgccggcatggccttcgcctccggaaggtactctcactctctgtccctcacacatacgcataaatggaaaggggttcatgtacataatttttttccccatacgatccaagaatggacgtgcaaatttctgattatgggcggttgatctgcgtcctttgaaggttctactgcatgagctcgtcgccgttcgcggttctcgtgttcgacgtggcggcgaacgtctggagcaaggtgcagccgccgatgaggcggttcctgcagtcgccggcgctggtcgagctcggcggcggcagggagggctcgggcaccgcaagggtggggctcgtcgcgtccgtggagaagagccgtctcagcgtgccgcggagcgtgcgcgtctggacactgcgcggcagaggaggctccggcggcggcggcggcgcgtggagcgaggtggcgcggatgccgcaggacgtgcacgcgcagttcgcggcggcggagggcggccgcgggttcgagtgcgcagcgcacggcgacttcgtcgcgctagcgccccgcggcgggccggcagccgtgccggtgccgacgaccgtgctcgtgttcgactcgcgccgcgacgagtggcggtgggcgccaccatgcccatacgtcgggcacggcatggccgcagtggtcaacggcggaggcgcggggttccgggtcctcgcgtacgagccacgcctggcgacgccggccatcggccttctggacgccacgacgccggtggctttgcatgggatgcatggttag

SEQ ID NO: 3 - protein, 440 aa, Triticum aestivum
  VARIANT (47)..(47): F=C in Robigus
  VARIANT (384)..(384): D=N in Robigus
MNLLPHHHLSLPSGPGRRPSSAAEAVEMDPRVWRRLPQPLLDRVLAFLPTPSFLRARAVCRRFYHLLFSSPFLHSHLLHSPHLPFFAFAVPSAGHLLLLDPTSQPQGPSWFLLPLPIPGPAAGFSPAPASAGLLAFLSDASGHKTLLLANPITRLLAALPLGPTQRLSPTVGLAAGSTSIIAVVAGDDLVSPFAVKNISVDTFVADAASVPSSGFWAPSSLLPRLSSLDPRAGMAFASGRFYCMSSSPFAVLVFDVAANVWSKVQPPMRRFLQSPALVELGGGREGSGTARVGLVASVEKSRLSVPRSVRVWTLRGRGGSGGGGGAWSEVARMPQDVHAQFAAAEGGRGFECAAHGDFVALAPRGGPAAVPVPTTVLVFDSRRDEWRWAPPCPYVGHGMAAVVNGGGAGFRVLAYEPRLATPAIGLLDATTPVALHGMHG

SEQ ID NO: 4 - DNA, 4999 bp, Triticum aestivum
actcccttcgtctctaaatataagccttttaaatatttcaatagggactacatacaaagcaaaatgagtgaatttacactctaaaatatgtctatatacattcgtatgtagtttgtattgaaatctctaaaaggcttatatttagaaacggagggagtaatacaaaatcacaaagacgaatcacacatgtcattggctacaacagtggtcagctcattttttggcctcaaccgatagctgagcttaacacagtaagttcttgatgttttcctctactatttgaaatttaataaatcaaaatgcaaacttccgaaaatatattaattacctgtaaaaaaatcagattttcttttagtaatgtaatataccctcctgagtactctcaaaaagtatgagcatattttgatgtacgatttgattttttgggggatttttatctactttttggccaaatttgaagttttttaaacttcaaattgctagacaacaatgtgctcaaacttacccaaaatattcaacttaggtatgttgaattaccagaaccgttcaatttttttttaaatggactttgacttcaaatttgattgaaaattacataaaatctccagactttcaaatgactggaaagtttttaatttgcatgaaaccgacgggaaaagttgtgcttgaaattttcaaaacttatgaaatatagattgtaccgatagattttttgcccatttgtcttgaaactttgttcaattttggcaaaaataataattaagataacaatcatttatttcagaacttcatgacctggctatgctcaagctcacctatcagttcgagctgaaaatgagccacgcagtgatttagaaatttagtcaacgacatgtgggccctattgtcatattttgctttagttgattaattaatcgactcggtgatgtgatttttgttactacgagcaagtcattgttgtagtcagacctcatagttaaggtatcataggtagcatcatacggtcttatgcaataccaactaagcatattaatgatgtggcataatattaattaagaaaaaataatatgttagtatcatatccgcatcgtaataaatgttttactagtatacgtgtcttgtataacaataaacaaggtcaatctaaaatactaacctatgatacgatgcactatgaagatcacattagtattatatacatgatgctactgtactatgactccatgagcagcctcggtgaaatgtgagccccctctgttaaattatgttttaattgaataattgactcaagttattgttatgattgagagggcgagcgggctaatttgttctcgtttggccttcctctcacacgactaaaatgttgaactcaacatttttttagcttgtaaacagtagtgttaggtgtttctaaaaatacctacaaagccgtttggttctaaggcttgctatgtcatatcttatcacatttttgggttaaacttgtctaagattagttcaataaaatgaaagccacaacttgacaggcctaagcgaattttggcacactttctaagcgtatgcgacctgaggctctagtgcggaaaaacatatctcgcctaagataaggctttaaatcaaatattctcttaaggtggttaaatttgtctaagattagatgcggcagtccaaggcaatcacaaatcaaacatactttgaatgtgaggtgtaggagaaaatcaactatacatgcatggatggtgctgcaggttggttactggcattagcttgagcattgcgtccttgttactcatcttttactcttacacgacagcttgacaagtacctatcgattagcttgctatcgtgtcatccttgcaagcagattgaaccgtgggcagacagagacgtgtgctgttaggacttgggggcattttagaaagggagaatcactgtgcaaaaatggaaatggagtaacacgaaaatgccgggagaagacgggagggacagcgacaatagagcgcaaatggtaacaggcaaaccggttggtgctcggcgatgggggtgacgatgtatgggcaggcaggcatgcaggtctctctctctctctctctccatctcagtatctcacagtgacagccttttggaatagaaagcctgatggccctgtgataggcttcgagttggctgcttcatgcatgtcaattcgttggagctagagttcggcagctaggaagtgccctagctagctggctaggtttagccacagccgtagccactactcctgcactgcactgtactgtccaaagtactagtgtggttgtcctccacggaagcaacaaaattcctctcaccaagcaacgagatgcatggctctaggttggagcgaatggccggatcctagggaaattttctcctgcacctctttgtaaataaagagcatttgtaggtcttaaatttaactcctcaaaggctcaaacgacctttcaaaacctaactcctcaaatttgaggagtacaacgtgtgggtcatttcctaaaatttaactccgtaattctttatttttggataacttacacattcttttgcatacaaaatcaatttaatatatatttcacaccattgtataaacatcgtactgaaaaaaattggtcattttgtcgaacaccatgtggcgtgtgcgtcggcagccaaaggtgacgattgttgcaggagcctgggaggtgacgataggcaatggcgtggccattaatgggcagggacgtgggagcgggggcggcgcggctgctcacattttgaggtgtcaatcttgtcaagcttctcaaatttgagtcggcggggacaacttctcaaatatttgaggcattcaaggttttgtgaagggctaggtggagtttaactccacaaactctattttacgtgtttgattattaatacctgaaaattttgcttgcgtcagtcaattccaccaaggctgaagatgggggcgagcgccgccggctgcgagtggcggaggcaggcagatcggccggtggtatgtagcgagggaacgagagacgtgaacgcatatagaggaggagggtgaacagtaacaaacggacgaagaagatgcaacgtggaatgttggattttaatttttattgactgaagtaattttttattttcggacgactaagggataagtggtccatttctttttaagaaaataaagagatttgaggaaagtgctacgtgaaccattcatgtgttctgtggatatgtttgattaagtgcatttacctcgaattaagttgtccgattacatatggtgtttgattggttgcatcaaccttcgggtacacctctccccagcaagatctcgtgtaaacatcaaatctattggagatgctctaagaagatgcaagaaaccgtatgtgctctctcttaatccgtgaaaagagcaatgatgcaaaataaggttatcccttgggcctgaggaaacaaagaagatacatcaaagtccttgggggtgatcatcttcctcgtcctgaacgggctgatggtgtgccgctgttgttgcctctccatcgcggagccagcctgtcattattgattatgaaagggaagtcatcaattccacagttttgccagacaagcgtcctggagagaaactatcaaagcaaatgttgcggaagatccacagatccaaaatccaacctccaaaaaaactactatgtagccacttgggagtatttggaacaaaataaatatagttcctcggattcagagtatttgggagtaacaagagagtttctgatgaattttgaagcattggagccatatcaaactttttctatgtagccacttggatacaaaatattagacttctatggaccatataaaaattatgtgcagcatccataagggttgttctggtgtttcaccttggactatatcttaataaggacacataagcaaaaacctggtttggcatcctcttttttttctacaaaaggcatcctttttaatgaggaaagatagagttaccgtatcttaatccagatatagctcttagcatcaaaatcagaacaattcataaattttctatcacaaacatattgaatgaaagttccttttagatacaacaatgcaatccattagaggagctatatatatctaaataaatatttaatgcacatcatatagtttaagatataatgcctagggaattgttcgaattaagacatcatacaatttgaaaagaaacattctcattggaaattgttcgatcccggcaagcgtgaaaagaaaagaccatctccgcctcaattgtgtgaaatacttgttctagtcgattctttcctcagttcttgattgaaatatgacgattgaccttcttcttcaaccacccgatgtgcacggttcctcttcctgctcataaacaatttactctagttgcatcccaacatcgtgcccctacctcgcctccggctaggtcattccaagccctagtcgccgacgtcgcaaccctgtctcatgctcggcggctatctaattcgaggggcagcggaaacgaaggccaccaccacccacccacctacgccggtcaacactcgtcctcgcctccggacacgatgccgcaactaaccacatcatacccagccgcgtcgacccctctttggcctctgagcttaggcaactgataaatagcaaacgcggaaaggaaaaccctaaggcaaacttagaactgccaaaacgaacatttataagtttttcttcatcaagatcaaatgcagaggagatcaatgtttttaggacaataggagagaccattgttcaaaaaaaaattaggggagaccagtgtacatggttcattaactcaacctatcagctagctaggctcctcattgcaagtggagtatttcttgtgccctcttctcctccccggttcccccacttcactcctgcagctcagctcactcactctcactccacgcacttccgggccagctccctgccactctccagctctccgctcacg

SEQ ID NO: 5 - DNA, 4997 bp, Triticum aestivum
actcccttcgtctctaaatataagccttttaaatatttcaatagggactacatacaaagcaaaatgagtgaatttacactctaaaatatgtctatatacattcgtatgtagtttgtattgaaatctctaaaaggcttatatttagaaacggagggagtaatacaaaatcacaaagacgaatcacacatgtcattggctacaacagtggtcagctcattttttggcctcaaccgatagctgagcttaacacagtaagttcttgatgttttcctctactatttgaaatttaataaatcaaaatgcaaacttccgaaaatatattaattacctgtaaaaaaatcagattttcttttagtaatgtaatataccctcctgagtactctcaaaaagtatgagcatattttgatgtacgatttgattttttgggggatttttatctactttttggccaaatttgaagttttttaaacttcaaattgctagacaacaatgtgctcaaacttacccaaaatattcaacttaggtatgttgaattaccagaaccgttcaatttttttttaaatggactttgacttcaaatttgattgaaaattacataaaatctccagactttcaaatgactggaaagtttttaatttgcatgaaaccgacgggaaaagttgtgcttgaaattttcaaaacttatgaaatatagattgtaccgatagattttttgcccatttgtcttgaaactttgttcaattttggcaaaaataataattaagataacaatcatttatttcagaacttcatgacctggctatgctcaagctcacctatcagttcgagctgaaaatgagccacgcagtgatttagaaatttagtcaacgacatgtgggccctattgtcatattttgctttagttgattaattaatcgactcggtgatgtgatttttgttactacgagcaagtcattgttgtagtcagacctcatagttaaggtatcataggtagcatcatacggtcttatgcaataccaactaagcatattaatgatgtggcataatattaattaagaaaaaataatatgttagtatcatatccgcatcgtaataaatgttttactagtatacgtgtcttgtataacaataaacaaggtcaatctaaaatactaacctatgatacgatgcactatgaagatcacattagtattatatacatgatgctactgtactatgactccatgagcagcctcggtgaaatgtgagccccctctgttaaattatgttttaattgaataattgactcaagttattgttatgattgagagggcgagcgggctaatttgttctcgtttggccttcctctcacacgactaaaatgttgaactcaacatttttttagcttgtaaacagtagtgttaggtgtttctaaaaatacctacaaagccgtttggttctaaggcttgctatgtcatatcttatcacatttttgggttaaacttgtctaagattagttcaataaaatgaaagccacaacttgacaggcctaagcgaattttggcacactttctaagcgtatgcgacctgaggctctagtgcggaaaaacatatctcgcctaagataaggctttaaatcaaatattctcttaaggtggttaaatttgtctaagattagatgcggcagtccaaggcaatcacaaatcaaacatactttgaatgtgaggtgtaggagaaaatcaactatacatgcatggatggtgctgcaggttggttactggcattagcttgagcattgcgtccttgttactcatcttttactcttacacgacagcttgacaagtacctatcgattagcttgctatcgtgtcatccttgcaagcagattgaaccgtgggcagacagagacgtgtgctgttaggacttgggggcattttagaaagggagaatcactgtgcaaaaatggaaatggagtaacacgaaaatgccgggagaagacgggagggacagcgacaatagagcgcaaatggtaacaggcaaaccggttggtgctcggcgatgggggtgacgatgtatgggcaggcaggcatgcaggtctctctctctctctctccatctcagtatctcacagtgacagccttttggaatagaaagcctgatggccctgtgataggcttcgagttggctgcttcatgcatgtcaattcgttggagctagagttcggcagctaggaagtgccctagctagctggctaggtttagccacagccgtagccactactcctgcactgcactgtactgtccaaagtactagtgtggttgtcctccacggaagcaacaaaattcctctcaccaagcaacgagatgcatggctctaggttggagcgaatggccggatcctagggaaattttctcctgcacctctttgtaaataaagagcatttgtaggtcttaaatttaactcctcaaaggctcaaacgacctttcaaaacctaactcctcaaatttgaggagtacaacgtgtgggtcatttcctaaaatttaactccgtaattctttatttttggataacttacacattcttttgcatacaaaatcaatttaatatatatttcacaccattgtataaacatcgtactgaaaaaaattggtcattttgtcgaacaccatgtggcgtgtgcgtcggcagccaaaggtgacgattgttgcaggagcctgggaggtgacgataggcaatggcgtggccattaatgggcagggacgtgggagcgggggcggcgcggctgctcacattttgaggtgtcaatcttgtcaagcttctcaaatttgagtcggcggggacaacttctcaaatatttgaggcattcaaggttttgtgaagggctaggtggagtttaactccacaaactctattttacgtgtttgattattaatacctgaaaattttgcttgcgtcagtcaattccaccaaggctgaagatgggggcgagcgccgccggctgcgagtggcggaggcaggcagatcggccggtggtatgtagcgagggaacgagagacgtgaacgcatatagaggaggagggtgaacagtaacaaacggacgaagaagatgcaacgtggaatgttggattttaatttttattgactgaagtaattttttattttcggacgactaagggataagtggtccatttctttttaagaaaataaagagatttgaggaaagtgctacgtgaaccattcatgtgttctgtggatatgtttgattaagtgcatttacctcgaattaagttgtccgattacatatggtgtttgattggttgcatcaaccttcgggtacacctctccccagcaagatctcgtgtaaacatcaaatctattggagatgctctaagaagatgcaagaaaccgtatgtgctctctcttaatccgtgaaaagagcaatgatgcaaaataaggttatcccttgggcctgaggaaacaaagaagatacatcaaagtccttgggggtgatcatcttcctcgtcctgaacgggctgatggtgtgccgctgttgttgcctctccatcgcggagccagcctgtcattattgattatgaaagggaagtcatcaattccacagttttgccagacaagcgtcctggagagaaactatcaaagcaaatgttgcggaagatccacagatccaaaatccaacctccaaaaaaactactatgtagccacttgggagtatttggaacaaaataaatatagttcctcggattcagagtatttgggagtaacaagagagtttctgatgaattttgaagcattggagccatatcaaactttttctatgtagccacttggatacaaaatattagacttctatggaccatataaaaattatgtgcagcatccataagggttgttctggtgtttcaccttggactatatcttaataaggacacataagcaaaaacctggtttggcatcctcttttttttctacaaaaggcatcctttttaatgaggaaagatagagttaccgtatcttaatccagatatagctcttagcatcaaaatcagaacaattcataaattttctatcacaaacatattgaatgaaagttccttttagatacaacaatgcaatccattagaggagctatatatatctaaataaatatttaatgcacatcatatagtttaagatataatgcctagggaattgttcgaattaagacatcatacaatttgaaaagaaacattctcattggaaattgttcgatcccggcaagcgtgaaaagaaaagaccatctccgcctcaattgtgtgaaatacttgttctagtcgattctttcctcagttcttgattgaaatatgacgattgaccttcttcttcaaccacccgatgtgcacggttcctcttcctgctcataaacaatttactctagttgcatcccaacatcgtgcccctacctcgcctccggctaggtcattccaagccctagtcgccgacgtcgcaaccctgtctcatgctcggcggctatctaattcgaggggcagcggaaacgaaggccaccaccacccacccacctacgccggtcaacactcgtcctcgcctccggacacgatgccgcaactaaccacatcatacccagccgcgtcgacccctctttggcctctgagcttaggcaactgataaatagcaaacgcggaaaggaaaaccctaaggcaaacttagaactgccaaaacgaacatttataagtttttcttcatcaagatcaaatgcagaggagatcaatgtttttaggacaataggagagaccattgttcaaaaaaaaattaggggagaccagtgtacatggttcattaactcaacctatcagctagctaggctcctcattgcaagtggagtatttcttgtgccctcttctcctccccggttcccccacttcactcctgcagctcagctcactcactctcactccacgcacttccgggccagctccctgccactctccagctctccgctcacg

SEQ ID NO: 6 - DNA, 1323 bp, Triticum aestivum
atgaacctactgcctcaccaccacctgtcgctgccgtctgggcctggccgccgcccctcctctgcggcggaggcggtggagatggacccgcgcgtgtggcgccgcctgccgcagccgctgctggaccgcgtgctggcgtgcctcccgacgccgtccttcctccgcgcccgcgccgtctgccgccgcttctaccacctcctcttctcctccccgttcctccactctcacctcctccactccccgcacctccccttcttcgccttcgccgtcccctccgccggccacctcctcctcctcgatcccacctcccagccgcagggaccctcctggttcctcctcccgctcccgatcccaggtcccgccgcggggttctcgccggctcccgcgtccgctggcctgctggcgttcctctccgacgcgtccggccacaagacgctgctcctcgccaaccccatcacgcgcctcctcgccgcgctgccgctcggccccacgcagcgcctctcccccaccgtcggcctggccgcggggtcgacgtccatcatcgccgtcgtggctggcgacgacctcgtgtcccctttcgccgtcaagaacatctccgtcgacaccttcgtcgccgacgccgcctccgtcccgtcctccggcttctgggcccccagctccctcctgccacgcctgtcctccctcgatcctcgcgccggcatggccttcgcctccggaaggttctactgcatgagctcgtcgccgttcgcggttctcgtgttcgacgtggcggcgaacgtctggagcaaggtgcagccgccgatgaggcggttcctgcagtcgccggcgctggtcgagctcggcggcggcagggagggctcgggcaccgcaagggtggggctcgtcgcgtccgtggagaagagccgtctcagcgtgccgcggagcgtgcgcgtctggacactgcgcggcagaggaggctccggcggcggcggcggcgcgtggagcgaggtggcgcggatgccgcaggacgtgcacgcgcagttcgcggcggcggagggcggccgcgggttcgagtgcgcagcgcacggcgacttcgtcgcgctagcgccccgcggcgggccggcagccgtgccggtgccgacgaccgtgctcgtgttcgactcgcgccgcaacgagtggcggtgggcgccaccatgcccatacgtcgggcacggcatggccgcagtggtcaacggcggaggcgcggggttccgggtcctcgcgtacgagccacgcctggcgacgccggccatcggccttctggacgccacgacgccggtggctttgcatgggatgcatggttag

SEQ ID NO: 7 - DNA, 1457 bp, Triticum aestivum
atgaacctactgcctcaccaccacctgtcgctgccgtctgggcctggccgccgcccctcctctgcggcggaggcggtggagatggacccgcgcgtgtggcgccgcctgccgcagccgctgctggaccgcgtgctggcgtgcctcccgacgccgtccttcctccgcgcccgcgccgtctgccgccgcttctaccacctcctcttctcctccccgttcctccactctcacctcctccactccccgcacctccccttcttcgccttcgccgtcccctccgccggccacctcctcctcctcgatcccacctcccagccgcagggaccctcctggttcctcctcccgctcccgatcccaggtcccgccgcggggttctcgccggctcccgcgtccgctggcctgctggcgttcctctccgacgcgtccggccacaagacgctgctcctcgccaaccccatcacgcgcctcctcgccgcgctgccgctcggccccacgcagcgcctctcccccaccgtcggcctggccgcggggtcgacgtccatcatcgccgtcgtggctggcgacgacctcgtgtcccctttcgccgtcaagaacatctccgtcgacaccttcgtcgccgacgccgcctccgtcccgtcctccggcttctgggcccccagctccctcctgccacgcctgtcctccctcgatcctcgcgccggcatggccttcgcctccggaaggtactctcactctctgtccctcacacatacgcataaatggaaaggggttcatgtacataatttttttccccatacgatccaagaatggacgtgcaaatttctgattatgggcggttgatctgtgtcctttgaaggttctactgcatgagctcgtcgccgttcgcggttctcgtgttcgacgtggcggcgaacgtctggagcaaggtgcagccgccgatgaggcggttcctgcagtcgccggcgctggtcgagctcggcggcggcagggagggctcgggcaccgcaagggtggggctcgtcgcgtccgtggagaagagccgtctcagcgtgccgcggagcgtgcgcgtctggacactgcgcggcagaggaggctccggcggcggcggcggcgcgtggagcgaggtggcgcggatgccgcaggacgtgcacgcgcagttcgcggcggcggagggcggccgcgggttcgagtgcgcagcgcacggcgacttcgtcgcgctagcgccccgcggcgggccggcagccgtgccggtgccgacgaccgtgctcgtgttcgactcgcgccgcaacgagtggcggtgggcgccaccatgcccatacgtcgggcacggcatggccgcagtggtcaacggcggaggcgcggggttccgggtcctcgcgtacgagccacgcctggcgacgccggccatcggccttctggacgccacgacgccggtggctttgcatgggatgcatggttag

SEQ ID NO: 8 - protein, 440 aa, Triticum aestivum
MNLLPHHHLSLPSGPGRRPSSAAEAVEMDPRVWRRLPQPLLDRVLACLPTPSFLRARAVCRRFYHLLFSSPFLHSHLLHSPHLPFFAFAVPSAGHLLLLDPTSQPQGPSWFLLPLPIPGPAAGFSPAPASAGLLAFLSDASGHKTLLLANPITRLLAALPLGPTQRLSPTVGLAAGSTSIIAVVAGDDLVSPFAVKNISVDTFVADAASVPSSGFWAPSSLLPRLSSLDPRAGMAFASGRFYCMSSSPFAVLVFDVAANVWSKVQPPMRRFLQSPALVELGGGREGSGTARVGLVASVEKSRLSVPRSVRVWTLRGRGGSGGGGGAWSEVARMPQDVHAQFAAAEGGRGFECAAHGDFVALAPRGGPAAVPVPTTVLVFDSRRNEWRWAPPCPYVGHGMAAVVNGGGAGFRVLAYEPRLATPAIGLLDATTPVALHGMHG

SEQ ID NO: 9 - DNA, 4886 bp, Triticum aestivum
actcccttcgtctctaaatataagccttttaggtatttcaatagggactacatacaaagcaaaatgagtgaatttacactctaaaatatgtctatatacattcgtatgtagtttgtattgaaatctctaaaaggcttatatttagaaacggagggagtaatacaaaatcacaaagacgaatcacacatgtcattggctacaacagtggtcagctcattttttggcctcaaccgatagctgagcttaacacagtaagttcttgatgttttcctctactatttgaaatttaataaatcaaaatgcaaacttccgaaaatatattaattacctgtaaaaaaatcagattttcttttagtaatgtaatataccctcctgagtactctcaaaaagtatgagcatattttgatgtacgatttgattttttgggggatttttatctactttttggccaaatttgaagttttttaaacttcaaattgctagacaacaatgtgctcaaacttacccaaaatattcaacgtaggtatgttgaattaccagaaccgttcaattttttttaaatggactttgacttcaaatttgattgaaaattacataaaatctccagactttcaaatgactggaaagtttttaatttgcatgaaaccgacaggaaaagttgtgcttgaaattttcaaaacttatgaaatatagattgtaccgatagattttttgcccatttgtcttgaaactttgttcaattttggcaaaaataataattaagataacaatcatttatttcagaacttcatgacctggctatgctcaagctcacctatcagttcgagctgaaaatgagccacgcagtgatttagaaatttagtcaacgacatgtgggccctattgtcatattttgctttagttgattaattaatcgactcggtgatgtgatttttgttactacgagcaagtcattgttgtagtcagacctcatagttaaggtatcataggtagcatcatacggtcttatgcaataccaactaagcatattaatgatgtggcataatattaattaagaaaaaaataatatgttagtatcatatccgcatcgtaataaatgttttactagtatacgtgtcttgtataacaataaacaaggtcaatctaaaatactaacctatgatacgatgcactatgaagatcacattagtattatatacatgatgctactgtactatgactccatgagcagcctcggtgaaatgtgagccccctctgttaaattatgttttaattgaataattgactcaagttattgttatgattgagagggcgagcgggctaatttgttctcgtttggccttcctctcacacgactaaaatgttgaactcaacatttttttagcttgtaaacagtagtgttaggtgtttctaaaaatacctacaaagccgtttggttctaaggcttgctatgtcatatcttatcacatttttgggttaaatttgtctaagattagttcaataaaatgaaagccacaacttgacaggcctaagcgaattttggcacactttctaagcgtatgcgacctgaggctctagtgcggaaaaacatatctcgcctaagataaggctttaaatcaaatattctcttaaggtggtcaaatttgtctaagattagatgcggcagtccaaggcaatcacaaatcaaacatactttgaatgtgaggtgtaggagaaaatcaactatacatgcatggatggtgctgcaggttggttactggcattagcttgagcattgcgtccttgttactcatcttttactcttacacgacagcttgacaagtacctatcgattagcttgctatcgtgtcatccttgcaagcagattgaaccgtgggcagacagagacgtgtgctgttaggacttgggggcattttagaaagggagaatcactgtgcaaaaatggaaatggagtaacacgaaaatgccgggagaagacgggagggacagcgacaatagagcgcaaatggtaacaggcaaaccggttggtgctcggcgatgggggtgacgatgtatgggcaggcaggcatgcaggtctctctctctctctctctctctccatctcagtatctcacagtgacagccttttggaatagaaagcctgatggccctgtgataggcttcgagttggctgcttcatgcatgtcaattcgttggagctagagttcggcagctaggaagtgccctagctagctggctaggtttagccacagccgtagccactactcctgcactgcactgtactgtccaaagtactagtgtggttgtcctccacggaagcaacaaaattcctctcaccaagcaacgagatgcatggctctaggttggagcgaatggccggatcctagggaaattttctcctgcacctctttgtaaataaagagcatttgtaggtcttaaatttaactcctcaaaggctcaaacgacctttcaaaacctaactcctcaaatttgaggagtacaacgtgtgggtcatttcctaaaatttaactccgtaattctttatttttggataacttacacattcttttgcatacaaaatcaatttaatatatatttcacaccattgtataaacatcgtactgaaaaaaattggtcattttgtcgaacaccatgtggcatgtgcgtcggcagccaaaggtgacgattgttgcaggagcctgggaggtgacgataggcaatggcgtggcaattaatgggcagggacatgggagcggggtcggcgcggctgctcacattttgaggcgtcaattttgtcaagcttctcaaatttgagtcggcggggacaacttctcaaatatttgaggcattcaaggttttgtgaagggctaggtggagtttaactccacaaactctattttacgtgtttgattattaatacctgaaaattttgcttgcgtcagtcaattccaccaaggctgaagatgggggcgagcgccgccggctgcgagtggcggaggtaggcagatcggccggtggtatgtagcgagggaacgagagacgtgaacgcatatagaggaggagggtgaacagtaacaaacggacgaagaagatgcaacgtggaatgttggattttaatttttattgactgaagtaattttttattttcggacgactaagggataagtggtccatttctttttaagaaaataaagagatttgaggaaagtgctacgtgaaccattcatgtgttctgtggatgtgtttgattaagtgcatttacctcgaattaagttgtccgattacatatggtgtttgattggttgcatcaaccttcgggtacacctctccccagcaagatctcgtgtaaacatcaaatctattggagatgctctaagaagatgcaagaaaccgtatgtgctctctcttaatccgtgaaaagagcaatgatgcaaaataaggttatcccttgggcccgaggaaacaaagaagatacatcaaagtccttgggggcgatcatcttcctcgtcctgaacgggctgatggtgtgccgctgttgttgcctctccatcgcggagccagcctgtcattattgattatgaaagggaagtcatcaattccacggttttgccagacaagcgtcctggagagaaactatcaaagcaaatgttgcggaagatccacagatccaaaatccaacctccaaaaaaactactatgtagccacttgggagtatttggaacaaaataaatatagttcctcggattcagagtatttgggagtaacaagagagtttctgatgaattttgaagcattggagccatatcaaactttttctatgtagccacttggatacaaaatattagacttctatggaccatataaaaattatgtgcagcatccataagggttgttctggtgtttcaccttggactatatcttaataaggacacataagcaaaaacctggtttggcatcctcttttttttctacaaaaggcatcctttttaatgaggaaagatagagttaccgtatcttaatccagatatagctcttagcatcaaaatcagaacaattcataaattttctatcacaaacatattgaatgaaagttccttttagatacaacaataatccattagaggagctatatatatctaaataaatatttaatgcacatcatatagtttaagatataatgcctagggaattgttcgaattaagacatcatacaatttgaaaagaaacattctcattggaaattgttcgatcccggcaagcgtgaaaagaaaagaccatctccgcctcaattgtgtgaaatacttgttctagtcgattctttcctcagttcttgattgaaatatgacgattgaccttcttcttcaaccacccgatgtgcacggttcctcttcctgctcataaaagaggggcagcggaaacgaaggccgccaccacccacccacctacgccggtcaacactcgtcctcgcctccggacacgatgccgcaactaaccacatcatacccagccgcgtcgacccctctttggcctctgagcttaggcaactgataaatagcaaacgcggaaaggaaaaccctaaggcaaacttagaactgccaaaacgaacatttataagtttttcttcatcaagatcaaatgcagaagagatcaatgtttttaggacaataggagagaccattgttcaaaaaaaaattaggggagaccagtgtacatggttcattaactcaacctatcagctagctaggctcctcagtgcaagtggagtatttcttgtgccctcttctcctccccggttcccccacttcactcctgcagctcagctcactcactctcactccacgcacttccgggccagctccctgccactctccagctctccgctcacg

SEQ ID NO: 10 - DNA, 201 bp, Triticum aestivum
ccccaatgttcgactcctgtgattcagaggactacactcgcacgagttacctcaatgatctggacaagcacaagcagcttgcgttggagcagtgatctttytagccgactttggacagattcggcatctttttgtacatcgtgactatcttcattgtgatcctcccgctttccagagatgtttatagagtgtatggataga

SEQ ID NO: 11 - DNA, 101 bp, Triticum aestivum
cccaagaagccgaaggcgacttgaaggaaatagtttaatacttcgtcgtgygttgtgacggtctgtcgttaattagtactaggtcctaggtatggtgatat

SEQ ID NO: 12 - DNA, 101 bp, Triticum aestivum
tcctccctgacctcgaggccgcgcaggcatggctcaccaagaacccgacckaggaacccgctgaggagccactaggggccgcgctgctgcgcaggatggcc

SEQ ID NO: 13 - DNA, 294 bp, Triticum aestivum
ggttgtgttgctgatccctttgaaactttctattttttggatcattggaccagttcgagttatcctaagtgccagccctaacarcaaagcattttatcatgtctagcaagcccaacaagactaatagggcacacatggacatgtggcggcggcgacaacaacgaaatcaacacggggctacgacgacgacgatggagacatcgacgtcgacaaggggctgcggaggcaacaacgacgacggcatcgtcgcggtgctgtgggggcggcgacgacgatggcatcatcacggtgcta

SEQ ID NO: 14 - DNA, 295 bp, Triticum aestivum
ggttgtgttgctgatccctttgaaactttctattttttggatcattggaccagttcgagttatcctaagtgccagccctaacaacaaagcattttatcatgtctagcaagcccaacaagactaatagggcacacatggacatgtggcggcggcgacaacaacgaaatcaacacggggctacgacgacracgatggagacatcgacgtcgacaaggggctgcggaggcaacaacgacgacggcatcgtcgcggtgctgtgggggcggcgacgacgatggcatcatcacggtgctac

SEQ ID NO: 15 - DNA, 1452 bp, Triticum aestivum
atgcaagtggagtatttcttgtgccctcttctcctcccggttccccctcttcacacctgcagctcagctcactcactctccctcacactccgggccagctgcctcccactctccatccctcagctcacgatgaacccacaccctcaccaccacctgtccctgccgtctgggcctggccgccgcccctcctctgcggcggaggcggtggagatggacccgcgggtgtggcgccggctgccgcagccgctgctggaccgcgtgctggcgtgcctcccgacgccgtccttcctccgcgcccgcgccgtctgccgccgcttctaccacctcctcttctcctccccattcctccactctcacctcctccactcgccgcacctccccttcttcgccttcgccgtcccctccgccggccacctcctcctcctcgaccccacctcccagccgcagggaccgtcctggttcctcctcccgctcccgatcccgggccccgccgcggggttctcgccggctgccgcggccgctggcctgctggcgtttctctccgacgcctccggccataaaacgctgctcctcgccaaccccatcacgcgcctccttgccgcgctgccgctcggccccacgcagcgcctctcccccaccgtcggcctggccgcggggtcgacgtccatcattgccgtcgtggctggcgacgacctcgtgtcccctttcgccgtcaagaatatctccgtcgacaccttcgtcgccgacgccgcctccgtcccgtcctccggcttctgggcccccagctccctcctgccacgcctgtcctccctcgatcctcgcgccggcatggccttcgcctccggaaggttctactgcatgagctcgtcgccgttcgcggttctcgtgttcgacgtggcggcgaacgtctggagcaaggtgcagccgccgatgaggcggttcctgcggtcgccggcgctggtggagctcggtggcggcagggagggctcgggcaccgcaagggtggggctcgtcgcgtccgtggagaagagccgtctcagcgtgccgcggagcgtgcgcgtctggacactgcgcggcagaggagcctccggcggcggcggcggcgcgtggagcgaggtggcgcggatgccgcaggacgtgcacgcgcagttcgcggccgcggagggcgggcgagggttcgagtgcgccgcccacggcgacttcgtcgtgctcgcgccccgcggcgggccggcagccatgccggtgccgacgaccgtgctggtgttcgactcgcgccgcgacgagtggcggtgggcgccaccatgcccatacgtcgggcacggcatggccgcagtggtcaacggcggaggcaacgggttccgggtcctcgcgtacgagccacgcctggcgacgccggccatcggccttctggatgccacgacgccggtggctttgcatgggatgcatggttag

SEQ ID NO: 16 - DNA, 1323 bp, Triticum aestivum
atgaacctactgcctcaccaccacctgtcgctgccgtctgggcctggccgccgcccctctccagcggcggcggaggtggagatggacccgcgcgtgtggcgccggctgccgcagccgctgctggaccgcgtgctggcgtgcctcccgacgccgtccttcctccgcgcccgcgccgtctgccgccgcttctaccacctcctcttctcctccccgttcctccactctcacctcctgcactccccgcacctccccttcttcgccttcgccgtcccctccgccggccacctcctcctcctcgaccccacctcccagccgcagggaccgtcctggttcctcctcccgctcccgatcccgggcccggccgcggggttctcgccggctgccgcttccgctggcctgctggcgttcctctccgacgcgtccgggcacaagacgctgctcctcgccaacccaatcacgcgcctcctcgccgcgctgccgctcggccccacgcagcgcctctcccccaccgtcggcctggccgcgggctcgacgtccatcatcgccgtcgtggctggcgacgacctcgtgtcccctttcgccgtcaagaacatctccgtcgacaccttcgtcgccgacgccgcctccgtcccgtcctccggcttctgggtccccagctccctcctgccacgcctgtcctccctcgaccctcgcgccggcatggccttcgcctccggaaggttctactgcatgagctcgtcgccgttcgcggttctcgtgttcgacgtggcggcgaacgtctggagcaaggtgcagccgccgatgaggcggttcctgcggtcgccggcgctggtggagctcggcggcggcagagagggctcgggcaccgcaagggtggggctcgtcgcgtccgtggagaagagccgtctcagcgtgccgaggagcgtgcgcgtctggacactgcgcggcagaggagcctccggcggcggtggcggcgcgtggagcgaggtggggcggatgccgcaggacgtgcacacgcagttcgcggccgcggagggcgggcgagggttcgagtgcgccgctcacggcgacttcgtcgtgctcgcgccccgcggcgggccggcaaccgtgccggtgccgacgaccgtgctcgtgttcgactcgcgccgcgacgagtggcggtgggcgccaccttgcccatacgtcgggcacggcatggccgcagtggccaacggcggaggcgcgggcttccgggtcctcgcgtacgagccacgcctggcgacgccggccatcggcctcctggacgccacgacgccggtggctttgcatgggatgcatggttag

SEQ ID NO: 17 - protein, 483 aa, Triticum aestivum
  VARIANT (47)..(47): H=R in Robigus
  VARIANT (173)..(173): A=S in Robigus
MQVEYFLCPLLLPVPPLHTCSSAHSLSLTLRASCLPLSIPQLTMNPHPHHHLSLPSGPGRRPSSAAEAVEMDPRVWRRLPQPLLDRVLACLPTPSFLRARAVCRRFYHLLFSSPFLHSHLLHSPHLPFFAFAVPSAGHLLLLDPTSQPQGPSWFLLPLPIPGPAAGFSPAAAAAGLLAFLSDASGHKTLLLANPITRLLAALPLGPTQRLSPTVGLAAGSTSIIAVVAGDDLVSPFAVKNISVDTFVADAASVPSSGFWAPSSLLPRLSSLDPRAGMAFASGRFYCMSSSPFAVLVFDVAANVWSKVQPPMRRFLRSPALVELGGGREGSGTARVGLVASVEKSRLSVPRSVRVWTLRGRGASGGGGGAWSEVARMPQDVHAQFAAAEGGRGFECAAHGDFVVLAPRGGPAAMPVPTTVLVFDSRRDEWRWAPPCPYVGHGMAAVVNGGGNGFRVLAYEPRLATPAIGLLDATTPVALHGMHG

SEQ ID NO: 18 - protein, 440 aa, Triticum aestivum
MNLLPHHHLSLPSGPGRRPSPAAAEVEMDPRVWRRLPQPLLDRVLACLPTPSFLRARAVCRRFYHLLFSSPFLHSHLLHSPHLPFFAFAVPSAGHLLLLDPTSQPQGPSWFLLPLPIPGPAAGFSPAAASAGLLAFLSDASGHKTLLLANPITRLLAALPLGPTQRLSPTVGLAAGSTSIIAVVAGDDLVSPFAVKNISVDTFVADAASVPSSGFWVPSSLLPRLSSLDPRAGMAFASGRFYCMSSSPFAVLVFDVAANVWSKVQPPMRRFLRSPALVELGGGREGSGTARVGLVASVEKSRLSVPRSVRVWTLRGRGASGGGGGAWSEVGRMPQDVHTQFAAAEGGRGFECAAHGDFVVLAPRGGPATVPVPTTVLVFDSRRDEWRWAPPCPYVGHGMAAVANGGGAGFRVLAYEPRLATPAIGLLDATTPVALHGMHG

SEQ ID NO: 19 - DNA, 8413 bp, Triticum aestivum
  misc_feature (900)..(901): insertion in Robigus
  misc_feature (6139)..(6148): CarG motif
  misc_feature (7816)..(7930): deletion in Robigus
cagactcaaatcggcgggtcggccacggctacgactctgctgccggcggcagggttggggatgggccgacgggcgacggcgacgcatttcagggcaaaaatcaccggcgggcgggctggcggaagtgcaacgaagacagttgggcagttgacgcgctgccgtctgttttacatctcctagagatgtagatgcaaatttgcaccgcaaggtgttgtagtttgtatcactgggaggcgaaaatgtaataaattttttttgcatctacaccatctgttgggaggtatttttggcctcagagatgcaaaagatagttattttgcatacgtctacgtagtgagatgggcatgtgcagtggaagcgagctggttcgcgtacgtctatgtagtgagatgggcatgtgctgggcaacacatcaatcaagctgccagtttgtgtgctttcgatctaacttatgtgcttcatctagctttgtttggatgaatcaatctagtgtggtctaaaactaacgagcagtatatgtttacatttccatgaagaaaagtatatgcttatatatataaaaagttggtgccaaatcaaggtgtggcggctgccacgccttgcccactggactcctccgccagtgagagggactgtgatatccctagcccaccagggttcaaattctggtgctcgcatttatttctggatttatttcaggatttccggcgatgcgcatttagtggaaggagacgtttctgtcgacgacgaggcgcctacggtgacttcgtaaatctcaagatgatatgccggttcagtctttcgaaggtgttcatagggataaggtgtgcgtgtgtgcattcataaaggtgagtgtatacacgtgtgtatgagcgcttgtgtactgatgttaaaaaaaaggccgcggcttgccatggacggtgcattggcgaatagctctcccgtagcgaggcacgcggaaggttctgccgttttgcatgcggcgatcatgcgtgcccccacccccgaccccccacggcgaagccccgtccgatcccacagctacgaagccagtgaagtacatagtgagctgtactggtgtagcctgcagggacggtatggtcaagagacgcaaggatggcacgcgcagcgtagcctttcaagcacaggcccgaatcatagggatgtgtgaccaaaaacaagtgccaggattctccctcttttgcatgtaaaccaaaggaaaggttagctccttcctgtctccctgcatccatgcgcccatgcatgcaagtggccactagcttgcagtgcagcaacgggatagtattaaaacgtctgctagggcgcaagggaacaaagcaaaagatatcatgatccacctgggagggaagtacgtgcaagcatccttctccctacctccgctgagccacgcacgagacccctacgtactccgtagagagccggtaggcaatacgtgagctctggtgctaaaccgatgccattgcgtcgtcgtaaaagctactccgtattagcctagcactatactatactgctcctataatatagcctgacggagaactactggtggtgacatcgaacatgtgtagtttgccctgtgacatcatatgtttttgggtctgtgttagtgcaaaactatgagggcatctccaatgcgaaccaacaaaccgctattaatctactgagctgtccaaacgccgcaagccatccaactttgtcctgcatttgttcgcggaccagtttgggagttcattttcccgtaaatcgaaagcaaagcaagggtggatatgcgagagtccggacacccgccatgaagcactccgacacccccggccctcccaaaaccaaggcggaccccgaacatttgaaccggtctgcaccttggttgttggttccatgagcacaagaattttgagctctaccgtgatgtgaaggaggaaaagaatgccaggagatttagcatcatgattggcggcgactaagaaggagacggatggtgatgaagaggaggcagcatagtgagtgcggaggctgaggtagccaatccggcagggcaaaaaaaaatccatttttctacatggactattggactgatacttctatgatcaggcgcatgtaaaaactatgacatctgcctctttttggctgtgatggacgctatgaaagccagactttaacttgctctaatattgctatgcatttgaaatgaaaatgggtggacctgaagggaggtttaggaaatgccattggatggccagcttccgcacccgtgtccgtgggctgctccgaaccagcccgcagaccaatacaatgttcgttttagggcatagcattagagatgccctaatagcagttttcaagtgatatcatatattctaaggttatgtagtaataaagaaaactcttggagaacctatttagcagattccccaaagctgtatttcagggcaaattttcctacatagaaaactaacctctagctagcataaaccctaaacatctattgatagtgtaaaaaaatggccttgcattttgggatggagggtaactctttacaattataagtttgcaccccttaaaataccaccttagcctatatttacttcaaggaggtcaactctcaagtaaaagtagggagatggcagtgtgtgccgccgccgttcatcttgcctccggcctaccccgctaaccatggcgcctcccctcagccctgcctccttccccccatcgtctctggtggcattccagaaagatactaacaatttttttcttcattttttgtctaccccaccatcttcgtccatggcggggcttctccccggcgtccctggcccaacggcgctcctctacccgccacaaggagaatagcaggacgagcagataaactcctactgaggagaaacatcatgtaaaatttttagttacatagaaacatggcagtcggcttccggctccgatttcaatgattcattgcgtttcatgcaacaccatcatgccatcaggagaaggaagacgagccattcagaagaagagtccaacccgtggctcacggaggacgaattttgcctactttctcgacttcttcttcgcatgcgctcggtgagccttcaaatttttacttttacaaatcatatatcctaggctctagctagctcatagacatcatctagtgtcatgaagtcatcactgaaccgcgtgaaaagacgacgaagaaagaagatgaagagcgccttggatgtgggggtcaaaccattatgtttaggacatctactagatgaagaaaaaaaattaattctgtaaaaaattgccttggtataagggatacgtttgggcaacatagtatagggaatctaccagagatgctcttagtcgagtcttttatttctgagagaactcataatcgagtccattagtcaactactcccttcgtctctaaatataagccttttaaatatttcaatagggactacatacaaagcaaaatgagtgaatttacactctaaaatatgtctatatacattcgtatgtagtttgtattgaaatctctaaaaggcttatatttagaaacggagggagtaatacaaaatcacaaagacgaatcacacatgtcattggctacaacagtggtcagctcattttttggcctcaaccgatagctgagcttaacacagtaagttcttgatgttttcctctactatttgaaatttaataaatcaaaatgcaaacttccgaaaatatattaattacctgtaaaaaaatcagattttcttttagtaatgtaatataccctcctgagtactctcaaaaagtatgagcatattttgatgtacgatttgattttttgggggatttttatctactttttggccaaatttgaagttttttaaacttcaaattgctagacaacaatgtgctcaaacttacccaaaatattcaacttaggtatgttgaattaccagaaccgttcaatttttttttaaatggactttgacttcaaatttgattgaaaattacataaaatctccagactttcaaatgactggaaagtttttaatttgcatgaaaccgacgggaaaagttgtgcttgaaattttcaaaacttatgaaatatagattgtaccgatagattttttgcccatttgtcttgaaactttgttcaattttggcaaaaataataattaagataacaatcatttatttcagaacttcatgacctggctatgctcaagctcacctatcagttcgagctgaaaatgagccacgcagtgatttagaaatttagtcaacgacatgtgggccctattgtcatattttgctttagttgattaattaatcgactcggtgatgtgatttttgttactacgagcaagtcattgttgtagtcagacctcatagttaaggtatcataggtagcatcatacggtcttatgcaataccaactaagcatattaatgatgtggcataatattaattaagaaaaaataatatgttagtatcatatccgcatcgtaataaatgttttactagtatacgtgtcttgtataacaataaacaaggtcaatctaaaatactaacctatgatacgatgcactatgaagatcacattagtattatatacatgatgctactgtactatgactccatgagcagcctcggtgaaatgtgagccccctctgttaaattatgttttaattgaataattgactcaagttattgttatgattgagagggcgagcgggctaatttgttctcgtttggccttcctctcacacgactaaaatgttgaactcaacatttttttagcttgtaaacagtagtgttaggtgtttctaaaaatacctacaaagccgtttggttctaaggcttgctatgtcatatcttatcacatttttgggttaaacttgtctaagattagttcaataaaatgaaagccacaacttgacaggcctaagcgaattttggcacactttctaagcgtatgcgacctgaggctctagtgcggaaaaacatatctcgcctaagataaggctttaaatcaaatattctcttaaggtggttaaatttgtctaagattagatgcggcagtccaaggcaatcacaaatcaaacatactttgaatgtgaggtgtaggagaaaatcaactatacatgcatggatggtgctgcaggttggttactggcattagcttgagcattgcgtccttgttactcatcttttactcttacacgacagcttgacaagtacctatcgattagcttgctatcgtgtcatccttgcaagcagattgaaccgtgggcagacagagacgtgtgctgttaggacttgggggcattttagaaagggagaatcactgtgcaaaaatggaaatggagtaacacgaaaatgccgggagaagacgggagggacagcgacaatagagcgcaaatggtaacaggcaaaccggttggtgctcggcgatgggggtgacgatgtatgggcaggcaggcatgcaggtctctctctctctctctccatctcagtatctcacagtgacagccttttggaatagaaagcctgatggccctgtgataggcttcgagttggctgcttcatgcatgtcaattcgttggagctagagttcggcagctaggaagtgccctagctagctggctaggtttagccacagccgtagccactactcctgcactgcactgtactgtccaaagtactagtgtggttgtcctccacggaagcaacaaaattcctctcaccaagcaacgagatgcatggctctaggttggagcgaatggccggatcctagggaaattttctcctgcacctctttgtaaataaagagcatttgtaggtcttaaatttaactcctcaaaggctcaaacgacctttcaaaacctaactcctcaaatttgaggagtacaacgtgtgggtcatttcctaaaatttaactccgtaattctttatttttggataacttacacattcttttgcatacaaaatcaatttaatatatatttcacaccattgtataaacatcgtactgaaaaaaattggtcattttgtcgaacaccatgtggcgtgtgcgtcggcagccaaaggtgacgattgttgcaggagcctgggaggtgacgataggcaatggcgtggccattaatgggcagggacgtgggagcgggggcggcgcggctgctcacattttgaggtgtcaatcttgtcaagcttctcaaatttgagtcggcggggacaacttctcaaatatttgaggcattcaaggttttgtgaagggctaggtggagtttaactccacaaactctattttacgtgtttgattattaatacctgaaaattttgcttgcgtcagtcaattccaccaaggctgaagatgggggcgagcgccgccggctgcgagtggcggaggcaggcagatcggccggtggtatgtagcgagggaacgagagacgtgaacgcatatagaggaggagggtgaacagtaacaaacggacgaagaagatgcaacgtggaatgttggattttaatttttattgactgaagtaattttttattttcggacgactaagggataagtggtccatttctttttaagaaaataaagagatttgaggaaagtgctacgtgaaccattcatgtgttctgtggatatgtttgattaagtgcatttacctcgaattaagttgtccgattacatatggtgtttgattggttgcatcaaccttcgggtacacctctccccagcaagatctcgtgtaaacatcaaatctattggagatgctctaagaagatgcaagaaaccgtatgtgctctctcttaatccgtgaaaagagcaatgatgcaaaataaggttatcccttgggcctgaggaaacaaagaagatacatcaaagtccttgggggtgatcatcttcctcgtcctgaacgggctgatggtgtgccgctgttgttgcctctccatcgcggagccagcctgtcattattgattatgaaagggaagtcatcaattccacagttttgccagacaagcgtcctggagagaaactatcaaagcaaatgttgcggaagatccacagatccaaaatccaacctccaaaaaaactactatgtagccacttgggagtatttggaacaaaataaatatagttcctcggattcagagtatttgggagtaacaagagagtttctgatgaattttgaagcattggagccatatcaaactttttctatgtagccacttggatacaaaatattagacttctatggaccatataaaaattatgtgcagcatccataagggttgttctggtgtttcaccttggactatatcttaataaggacacataagcaaaaacctggtttggcatcctcttttttttctacaaaaggcatcctttttaatgaggaaagatagagttaccgtatcttaatccagatatagctcttagcatcaaaatcagaacaattcataaattttctatcacaaacatattgaatgaaagttccttttagatacaacaatgcaatccattagaggagctatatatatctaaataaatatttaatgcacatcatatagtttaagatataatgcctagggaattgttcgaattaagacatcatacaatttgaaaagaaacattctcattggaaattgttcgatcccggcaagcgtgaaaagaaaagaccatctccgcctcaattgtgtgaaatacttgttctagtcgattctttcctcagttcttgattgaaatatgacgattgaccttcttcttcaaccacccgatgtgcacggttcctcttcctgctcataaacaatttactctagttgcatcccaacatcgtgcccctacctcgcctccggctaggtcattccaagccctagtcgccgacgtcgcaaccctgtctcatgctcggcggctatctaattcgaggggcagcggaaacgaaggccaccaccacccacccacctacgccggtcaacactcgtcctcgcctccggacacgatgccgcaactaaccacatcatacccagccgcctcgacccctctttggcctctgagcttaggcaactgataaatagcaaacgcggaaaggaaaaccctaaggcaaacttagaactgccaaaacgaacatttataagtttttcttcatcaagatcaaatgcagaggagatcaatgtttttaggacaataggagagaccattgttcaaaaaaaaattaggggagaccagtgtacatggttcattaactcaacctatcagctagctaggctcctcattgcaagtggagtatttcttgtgccctcttctcctccccggttcccccacttcactcctgcagctcagctcactcactctcactccacgcacttccgggccagctccctgccactctccagctctccgctcacg

SEQ ID NO: 20 - DNA, 1242 bp, Triticum aestivum
  variation (140)..(140): A=G in Robigus
  variation (517)..(517): G=T in Robigus
  variation (594)..(594): C=G in Robigus
  variation (762)..(762): G=A in Robigus
atggacccgcgggtgtggcgccggctgccgcagccgctgctggaccgcgtgctggcgtgcctcccgacgccgtccttcctccgcgcccgcgccgtctgccgccgcttctaccacctcctcttctcctccccattcctccactctcacctcctccactcgccgcacctccccttcttcgccttcgccgtcccctccgccggccacctcctcctcctcgaccccacctcccagccgcagggaccgtcctggttcctcctcccgctcccgatcccgggccccgccgcggggttctcgccggctgccgcggccgctggcctgctggcgtttctctccgacgcctccggccataaaacgctgctcctcgccaaccccatcacgcgcctccttgccgcgctgccgctcggccccacgcagcgcctctcccccaccgtcggcctggccgcggggtcgacgtccatcattgccgtcgtggctggcgacgacctcgtgtcccctttcgccgtcaagaatatctccgtcgacaccttcgtcgccgacgccgcctccgtcccgtcctccggcttctgggcccccagctccctcctgccacgcctgtcctccctcgatcctcgcgccggcatggccttcgcctccggaaggttctactgcatgagctcgtcgccgttcgcggttctcgtgttcgacgtggcggcgaacgtctggagcaaggtgcagccgccgatgaggcggttcctgcggtcgccggcgctggtggagctcggtggcggcagggagggctcgggcaccgcaagggtggggctcgtcgcgtccgtggagaagagccgtctcagcgtgccgcggagcgtgcgcgtctggacactgcgcggcagaggagcctccggcggcggcggcggcgcgtggagcgaggtggcgcggatgccgcaggacgtgcacgcgcagttcgcggccgcggagggcgggcgagggttcgagtgcgccgcccacggcgacttcgtcgtgctcgcgccccgcggcgggccggcagccatgccggtgccgacgaccgtgctggtgttcgactcgcgccgcgacgagtggcggtgggcgccaccatgcccatacgtcgggcacggcatggccgcagtggtcaacggcggaggcaacgggttccgggtcctcgcgtacgagccacgcctggcgacgccggccatcggccttctggatgccacgacgccggtggctttgcatgggatgcatggttag

SEQ ID NO: 21 - DNA, 1597 bp, Triticum aestivum
  exon 1 (1)..(844): exon
  variation (140)..(140): A=G in Robigus
  variation (517)..(517): G=T in Robigus
  variation (594)..(594): C=G in Robigus
  variation (762)..(762): G=A in Robigus
  exon 2 (990)..(1597)
atgcaagtggagtatttcttgtgccctcttctcctcccggttccccctcttcacacctgcagctcagctcactcactctccctcacactccgggccagctgcctcccactctccatccctcagctcacgatgaacccacaccctcaccaccacctgtccctgccgtctgggcctggccgccgcccctcctctgcggcggaggcggtggagatggacccgcgggtgtggcgccggctgccgcagccgctgctggaccgcgtgctggcgtgcctcccgacgccgtccttcctccgcgcccgcgccgtctgccgccgcttctaccacctcctcttctcctccccattcctccactctcacctcctccactcgccgcacctccccttcttcgccttcgccgtcccctccgccggccacctcctcctcctcgaccccacctcccagccgcagggaccgtcctggttcctcctcccgctcccgatcccgggccccgccgcggggttctcgccggctgccgcggccgctggcctgctggcgtttctctccgacgcctccggccataaaacgctgctcctcgccaaccccatcacgcgcctccttgccgcgctgccgctcggccccacgcagcgcctctcccccaccgtcggcctggccgcggggtcgacgtccatcattgccgtcgtggctggcgacgacctcgtgtcccctttcgccgtcaagaatatctccgtcgacaccttcgtcgccgacgccgcctccgtcccgtcctccggcttctgggcccccagctccctcctgccacgcctgtcctccctcgatcctcgcgccggcatggccttcgcctccggaaggtactcccgctctctctgtccctcacagatacgcataaatggaaaggggttcttgcacataacgtttttccccatacgatccaagaatggaccgggttcttgtgcaaatttctgattatgagctgctgatctgtgtcctttgaaggttctactgcatgagctcgtcgccgttcgcggttctcgtgttcgacgtggcggcgaacgtctggagcaaggtgcagccgccgatgaggcggttcctgcggtcgccggcgctggtggagctcggtggcggcagggagggctcgggcaccgcaagggtggggctcgtcgcgtccgtggagaagagccgtctcagcgtgccgcggagcgtgcgcgtctggacactgcgcggcagaggagcctccggcggcggcggcggcgcgtggagcgaggtggcgcggatgccgcaggacgtgcacgcgcagttcgcggccgcggagggcgggcgagggttcgagtgcgccgcccacggcgacttcgtcgtgctcgcgccccgcggcgggccggcagccatgccggtgccgacgaccgtgctggtgttcgactcgcgccgcgacgagtggcggtgggcgccaccatgcccatacgtcgggcacggcatggccgcagtggtcaacggcggaggcaacgggttccgggtcctcgcgtacgagccacgcctggcgacgccggccatcggccttctggatgccacgacgccggtggctttgcatgggatgcatggttag

SEQ ID NO: 22 - DNA, 3871 bp, Triticum aestivum
  misc_feature (2970)..(3380): n is a, c, g, or t
cacttgtatcaccatctcttcgccgaactagtacacctatacaatttaccattgtattggatgtgttggggacacaagagactctttgttatttggttgcagggttgtttgagagagaccaccttcatcctacgcctcccacggattgataaaccttaggtcttccacttgagggaaatctgctactgtcctacaaacctctgcacttggaggcccaacaatgtctacaagaagaaggttgcgtagtagacatcacttctctcaagacagaaaataatacggtgggtgaacaaattactgccgagcaattgatagaaagcgcaaagttatgatgatatctaaggaaatgatcatgaatatagacatcacgtccgtgtcaagtagaccgactcctgcctgcatctactactattactccatacatcgactgctatcgagcatgcatctagagtattaagttcataaaagaacggagtaacgcattaagtaagatggcatgatgtagaggaattaactcaagcagtatgacgaaaaccccatctttttatccccgatggcaacaatacaatacgtgccttgatgctcctactgtcactgggaaaggacaccgcaagattgaacccaaagctaagcacttctcccattgcaagaaaaaccaatctagttggccaaaccaaactgataattcgaagagaattacaaagatatcaaatcatgcatataatatttcagagaagattcaaataatattcatagataagctgatcatagatccataattcatcggatctcggcaaacacaccgccaaaaaagtattacatcgaatagatctccaagaacatcgaggagagcatggtattgagaatcaaagagagagaagaagccatctagctactagctatggacccgtaggtctgtggtaaactactcacgcttcatcggaagggaaatggtgttgatgtagaagccctccgtgatcaaatccccctccggcaggacgccggaaaaggcccctagatgggatctcatgggtatagaaggttacgacggcggaaaagtgttttcgtggatgcctctgttagtttggggatatatgggaatatataggcgaaagaattaggtcaggaggtgcacgaggggcccacaagggtgggggcacgccctccgtccttgtggccgcctcgtggctcctccaagtctcctggttgtcttctggtccaagaaaaatatcgcgaaggtttttttccgtttggactctgtttggtattccttttccgcaaagctcaaaaacagggaaaaaacaggaactggcactgggctctaggctaatagattagtcccaaaaataatataaaatagcatattaatgcagataaaacatccaaaacagataatataatagcatggaacaatcaaaaattatagatacattggagacgtatcaattccctttgtccatcggtatgttacttggccgagattcggtcatcgttatctttatacctagttcaatctcgctaccggcaaatctctttactctcgttctgtaatacatcacctcgtaactaaccccttagtcatttgcttgcaagcttatgatgtgtattaccgagagggcccagatatatacctctccgatactcggagtgacaaacccttatcttgatctatgccaactcaacaaacaccttcagagatgcatgtagagcctctttatgatgacccagttacgttgtgacatttgatagcacacaaggcattcctccggtattcgggagttgcataatctcatagtcgaaggaatatgtatttgacatgaagaaagcaatagcaataaactgaacgatcattatgctaagctaacggatgggtcttgcccatcacatcattcttctaatgatgtgatcccgttatcgaatgaaaactcatgtctatggttaggaaaccttaaccatgtttgatcaacgagctagtctagtagaggatcactaggaacacggtattagtttatgtattcacacatgtcattaagtttctgatcaatacaattctagcatgaataataaacgtttatcatgaataagaaaatataaaataactactttattattacctctagggcatatttctttcagaatgtgggaccgatattctcgttaactttatggcgatgttattttttgcccatacgtttctccctcccatctggtttgtaaggtccgcgcgtaattcaagaccgataattcggccagtgtaatataatatgagttatatatcataaaaaagttacactattacaaatttctttaggatacgaattcaatagtacattttgtgtgtcataagactaatattttattggtccaactaagagtgtaattttattgatactacacgtaattcaactaagagtagtatctagggacacattcccacacattgttaacctgaagagagggcgtattgcatagtaatgagtctagagttgcactataaagatgtttaaacatttttggaccattttgtgtctggtatttaagacatcatacaatgtagaaataaacaatcctgtaagaaagtgttagatcccggcaaacgtgaaaggaagggagcacctactcctcaattgtgtgatataatcgttctagtcgaatttttttctaagccgttgattgtaatatggcgactgtccttcattttcaacctcctgatgtgcacggttcctcttcctccttgcaacacaatttcgtctagtcgcagccaacgtcgtgcccaagcctcgcctccggcaaggtgttgtccaagctctcatcaaagacgttgcagcccatctcgttctcgacagctatctaacatgagtggcagcgaaaactaacccccccccccgcatccacccacacaggtcaaccctcgtccttgccttcatacgcgatgccacagcttatcctgtcgtccctgtcgccgatgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctctctctctctctctctctctctctctctctctctctctctctctccaagttgtatttcgaaaatgctaaaaatctcacgtcggatttttaggcattgcaatatgaatgttttttagggcaaattatgatgttacgaagatgagatatttgcttagcaagcatggcaattcctggcaaaaaattgaacatgacgatgatttaccatgtttgctaaaagaaattgccatccttatgacaatataatttgtcataaaaattgtcatcctcgcgacaacataatttgccataaaagacatttgatttctagcaaaatccctttcttaggcaacttagaattggcatagtaaccaacaataattaattaagtttttcccatcaagatcaaatgcagaagagatcaatgtttctaggacaatagcggagagccggagaccaatgtacaaggttcattaaaactcaaccatttagctagctagctaggctcctca

SEQ ID NO: 23 - DNA, 101 bp, Artificial Sequence
  marker
tagtaagctcttcaacgaggatggatgttgtgtaatttggacaagtgcgaygtatgtcacatcttttttttaatgatcctaatctatgatcgaagttcgtt

SEQ ID NO: 24 - DNA, 201 bp, Artificial Sequence
  marker
tgccggcctgcaagccgatccttactccaaartgggttgtctcggtgtttttccttgtcggcgtcgtctttgtcccagttggtgtcgtttcgctactagcygcacaagatgttgttgagatcattgatcggtatgatcatgcatgtgtcccacctaacatgactgataacaagcttgcgtacatccagaatgagactatac

SEQ ID NO: 25 - DNA, 101 bp, Artificial Sequence
  marker
tccacaagaaaagagcaagacactccggccgttgtagagctgatggtgcgyggtgatttcaccatagacatggtagacggcgcccgtcctcgtggcatcat

SEQ ID NO: 26 - DNA, 101 bp, Artificial Sequence
  marker
ggcacgtactccctttcaggacccgacgaacaacggcaattcaggtaaatrcatacatcacgtactcttacatacttcaatcttgtaaatccataatatat

SEQ ID NO: 27 - DNA, 101 bp, Artificial Sequence
  marker
atcccagggggcgagattcagagcttctcggccatcctgcgcagcagcgcrgcccctagtggctcctcggtcgggttcttggtgagccatgcctgcgcggc

SEQ ID NO: 28 - DNA, 1242 bp, Triticum aestivum
  variation (59)..(59): T=G in Robigus
  variation (1069)..(1069): G=A in Robigus
atggacccgcgcgtgtggcgccgcctgccgcagccgctgctggaccgcgtgctggcgttcctcccgacgccgtccttcctccgcgcccgcgccgtctgccgccgcttctaccacctcctcttctcctccccgttcctccactctcacctcctccactccccgcacctccccttcttcgccttcgccgtcccctccgccggccacctcctcctcctcgatcccacctcccagccgcagggaccctcctggttcctcctcccgctcccgatcccaggtcccgccgcggggttctcgccggctcccgcgtccgctggcctgctggcgttcctctccgacgcgtccggccacaagacgctgctcctcgccaaccccatcacgcgcctcctcgccgcgctgccgctcggccccacgcagcgcctctcccccaccgtcggcctggccgcggggtcgacgtccatcatcgccgtcgtggctggcgacgacctcgtgtcccctttcgccgtcaagaacatctccgtcgacaccttcgtcgccgacgccgcctccgtcccgtcctccggcttctgggcccccagctccctcctgccacgcctgtcctccctcgatcctcgcgccggcatggccttcgcctccggaaggttctactgcatgagctcgtcgccgttcgcggttctcgtgttcgacgtggcggcgaacgtctggagcaaggtgcagccgccgatgaggcggttcctgcagtcgccggcgctggtcgagctcggcggcggcagggagggctcgggcaccgcaagggtggggctcgtcgcgtccgtggagaagagccgtctcagcgtgccgcggagcgtgcgcgtctggacactgcgcggcagaggaggctccggcggcggcggcggcgcgtggagcgaggtggcgcggatgccgcaggacgtgcacgcgcagttcgcggcggcggagggcggccgcgggttcgagtgcgcagcgcacggcgacttcgtcgcgctagcgccccgcggcgggccggcagccgtgccggtgccgacgaccgtgctcgtgttcgactcgcgccgcgacgagtggcggtgggcgccaccatgcccatacgtcgggcacggcatggccgcagtggtcaacggcggaggcgcggggttccgggtcctcgcgtacgagccacgcctggcgacgccggccatcggccttctggacgccacgacgccggtggctttgcatgggatgcatggttag

SEQ ID NO: 29 - protein, 413 aa, Triticum aestivum
  VARIANT (20)..(20): F=C in Robigus
  VARIANT (357)..(357): D=N in Robigus
MDPRVWRRLPQPLLDRVLAFLPTPSFLRARAVCRRFYHLLFSSPFLHSHLLHSPHLPFFAFAVPSAGHLLLLDPTSQPQGPSWFLLPLPIPGPAAGFSPAPASAGLLAFLSDASGHKTLLLANPITRLLAALPLGPTQRLSPTVGLAAGSTSIIAVVAGDDLVSPFAVKNISVDTFVADAASVPSSGFWAPSSLLPRLSSLDPRAGMAFASGRFYCMSSSPFAVLVFDVAANVWSKVQPPMRRFLQSPALVELGGGREGSGTARVGLVASVEKSRLSVPRSVRVWTLRGRGGSGGGGGAWSEVARMPQDVHAQFAAAEGGRGFECAAHGDFVALAPRGGPAAVPVPTTVLVFDSRRDEWRWAPPCPYVGHGMAAVVNGGGAGFRVLAYEPRLATPAIGLLDATTPVALHGMHG

SEQ ID NO: 30 - DNA, 1242 bp, Triticum aestivum; identical to SEQ ID NO: 20
  variation (307)..(307): G=T in Robigus
  variation (384)..(384): C=G in Robigus
  variation (552)..(552): G=A in Robigus

SEQ ID NO: 31 - protein, 413 aa, Triticum aestivum
  VARIANT (103)..(103): A=S in Robigus
MDPRVWRRLPQPLLDRVLACLPTPSFLRARAVCRRFYHLLFSSPFLHSHLLHSPHLPFFAFAVPSAGHLLLLDPTSQPQGPSWFLLPLPIPGPAAGFSPAAAAAGLLAFLSDASGHKTLLLANPITRLLAALPLGPTQRLSPTVGLAAGSTSIIAVVAGDDLVSPFAVKNISVDTFVADAASVPSSGFWAPSSLLPRLSSLDPRAGMAFASGRFYCMSSSPFAVLVFDVAANVWSKVQPPMRRFLRSPALVELGGGREGSGTARVGLVASVEKSRLSVPRSVRVWTLRGRGASGGGGGAWSEVARMPQDVHAQFAAAEGGRGFECAAHGDFVVLAPRGGPAAMPVPTTVLVFDSRRDEWRWAPPCPYVGHGMAAVVNGGGNGFRVLAYEPRLATPAIGLLDATTPVALHGMHG

(19) United States
(12) Patent Application Publication
(10) Pub. No.: US 2020/0362366 A1
(43) Pub. Date: Nov. 19, 2020

(54) GENE UNDERLYING THE NUMBER OF SPIKELETS PER SPIKE QTL IN WHEAT ON CHROMOSOME 7A

(71) Applicants: BASF SE, Ludwigshafen am Rhein (DE); COMMONWEALTH SCIENTIFIC AND INDUSTRIAL RESEARCH ORGANISATION, Acton (AU)

(72) Inventors: Mark DAVEY, Gent (BE); Ruvini ARIYADASA, Gent (BE); Colin Robert CAVANAGH, Queensland (AU); Matthew HANNAH, Gent (BE); Xi WANG, Gent (BE); Lukas WITTERN, London (GB); Keith GARDNER, Cambridge (GB); William BOVILL, Acton (AU); Jose BARRERO SANCHEZ, Acton (AU); Klara VERBYLA, Acton (AU); Andrew SPRIGGS, Acton (AU); Alex ARUNDELL WEBB, Cambridge (GB)

(21) Appl. No.: 16/961,396

(22) PCT Filed: Jan. 11, 2019
(86) PCT No.: PCT/EP2019/050711

§ 371 Date: Jul. 10, 2020

(30) Foreign Application Priority Data
Jan. 12, 2018 (EP) 18151432.4
Jan. 12, 2018 (EP) 18151436.5

(51) Int. Cl. C12N 15/82; C07K 14/415; A21D 13/02; A23L 7/117

(52) U.S. Cl. CPC C12N 15/8262; C07K 14/415; A23V 2002/00; A23L 7/117; C12N 2510/00; A21D 13/02

(57) ABSTRACT

The present invention relates to the field of agriculture. In particular the invention provides a protein, a nucleic acid, a recombinant gene, plants comprising the recombinant gene and methods for altering the number of spikelets per spike of a wheat plant.

### FIELD OF THE INVENTION

The present invention concerns the field of plant optimization through molecular biology methods, marker technology and gene technology. Provided are technical means such as nucleic acid molecules, vectors and methods and uses thereof to produce and identify non-transgenic and transgenic wheat plants with altered “total spikelet number per spike” (“SPS” herein) phenotypes.

### BACKGROUND

Grain yield in wheat is determined predominantly by three yield components including productive spikes or ears per unit area, number of grains per spike and grain weight. One of the major factors that have contributed to wheat yield improvement is increase in kernels per spike or increase in both kernels per spike and number of spikes per unit area. The total kernel number may be further influenced by traits such as productive tillers per plant, spikelet number per spike, number of viable florets per spikelet. Gains in any of the yield components or traits can theoretically increase the yield potential of wheat. However, as these may compete for assimilates during spike growth stage, compensation effects may occur, and increase in one of the traits or components does not necessarily lead to an increase in total grain yield.

The genetics determining wheat inflorescence architecture remain largely unknown. Only the photoperiod sensitivity gene Ppd-1 has so far been shown to affect spikelet number [Shaw, L. M., et al., PLoS One, 2013. 8(11): p. e79459]. This represents a great source of untapped genetic potential to contribute to the efforts to meet the 70% crop yield increase needed by 2050 to feed a growing world population [United Nations, F.a.A.O.o.t.U. How to Feed the World in 2050. in Rome: High-Level Expert Forum. 2009]. The wheat inflorescence (commonly called the spike, ear or head) is composed of spikelets which are attached at rachis nodes. Each of the spikelets in turn is made up of two glumes and a number of florets of which usually two to four form a grain after fertilization. The final number of spikelets is determined by the formation of a terminal spikelet. This occurs when the last initiated primordia, instead of becoming spikelet primordia, develop into glume and floret primordia [Kirby, E. J. M. and M. Appleyard, F. G. H. Lupton, Editor. 1987, Springer Netherlands: Dordrecht. p. 287-311].

The development of permanent mapping populations in wheat in the last years, accompanied by the construction of genome-wide marker maps based on a large amount of molecular markers, opened the possibility to identify, analyze and use QTL for agronomical traits including spikelet number per spike.

Tian et al. (2015 Genetic analyses of wheat and molecular marker-assisted breeding volume 1 Science Press Beijing) summarize information for QTL related to spike morphology and (on page 167, Table 1.37) specifically for spikelet number. QTLs are identified on chromosome 2D, 2DS, 3AS, 3B, 3DL, 4AL, 4DS, 5A, 5B, 5D, 7A, 7AL and 7D.

Jantasuriyarat et al. (2004, Theor. Appl Genet. 108: 261-273) reported two QTL using recombinant inbred lines of the International Triticeae Mapping Initiative mapping population which were associated with spikelet number on chromosome 7A amongst other QTLs. One QTL as delimited by markers Xfba69-XksuH9 (182.7-213.4 cM—peak marker 196.3 cM—nearest locus Xmwg938) or as delimited by markers Xfba350-Xfbb18—188.5.3-201.3 cM—peak marker 196.3 cM—nearest locus Xmwg938) was significant on two locations in two years, while another was significant in one year on one location only (markers Xfbg354-Xfba350—160.1-174.9 cM—peak marker 164.9 cM—nearest locus Xfba69). Spikelet number was increased by alleles of Opata 85 in all cases.

Ma et al. (2007, Mol. Gen. Genomics 277: 31-42) reported two QTL for spikelet number per spike on chromosome 7A in a population of recombinant inbred lines (“RILs”) developed through single-seed descent from a cross between Nanda2419 and Wangshuibai, or in an immortalized F2 population generated by randomly permutated intermating of these RILs. In the RILs population, the QTL interval was delineated by markers Xbarc154-Xwmc83e, while in the IF2 population, the QTL was delineated by markers Xwmc83-Xwmc17. The Wangshuibai alleles contributed to more spikelets per spike.

Xu et al. (2014, Theor. Appl. Genet. 127: 59-72) reported the identification of a QTL for SPS on chromosome 7A in a population of RILs from a cross between Xiaoyan 54 and Jing 411) identified by markers Xgwm276-Xbarc192-Xbarc253. The parent Jing 411 contributed the favorable allele.

Zhai et al. (2016 Frontiers in Plant Science, Volume 7, article 1617) referred to the region on chromosome 7A identified by Xu et al. 2014, and indicated the region to be located between 123.50-137.50 cM interval.

Saarah Noriko Kuzay et al. (P0848 International Plant and Animal Genome XXV, Jan. 14-18, 2017 San Diego) referred in a poster abstract to the identification of a QTL for SPS on the long arm of chromosome 7AL using genome wide association studies. Validation of this QTL in the biparental population BerkutxRC875 allowed precise genetic mapping to a 2 Mb region of chromosome 7AL. On average, lines carrying the Berkut allele for SPS had 2.4 more spikelets per spike compared to the lines carrying the RAC875 allele for the peak region of the QTL. They also report development of a large high density population from two heterozygous inbred families to precisely map and eventually clone the gene underlying this QTL.

Zhang et al. (2015, Scientific Reports DOI 10:1038/srep12211) report that a putative MOC1 ortholog from wheat (MOC1 stands for MONCULM1 in rice) might be involved in wheat spikelet development. TaMoc1-A was mapped to a region flanked by WMC488 (4.7 cM) and P2071-180 (11.6 cM) on chromosome 7A in a population of doubled haploids from a cross between Hanxuan 10 and Lumai 14. TaMoc1-7A haplotype HapH was associated with a modest increase in spikelet number per spike in 10 environments over 3 years and 2 sites. However, this TaMOC1 orthologue is not the gene underlying the herein described QTL for SPS on chromosome 7A. Upon alignment of TaMOC1-7A to the NRgene-HiC reference genome of Chinese Spring (abbreviated herein at times as “CS”) wheat, TaMOC1-7A maps at 557,480,502 bp on chromosome 7A, which is more than 100 Mb distance from the herein described and analyzed 7A QTL for SPS and therefore appears to be different. As indicated below, the left and right markers identifying the QTL interval in the MAGIC mapping population map at 671,146,796 and 674,103,435 respectively, while the markers identifying the QTL interval in a GWAS study map at position 674,203,435 and 674,203,741 on wheat chromosome 7A (positions refer to the NRgene-HiC Chinese Spring reference genomic sequence).

There thus remains a need for further genetic dissection of the SPS QTL located on the chromosomes 7 of wheat, particularly 7A, to identify the underlying gene(s) in order to facilitate optimization of the number of spikelets per spike, in an attempt to achieve the maximum yield potential of wheat.

### SUMMARY OF THE INVENTION

In one aspect, the invention provides a protein involved in determining the number of spikelets per spike in wheat which is orthologous to “Aberrant panicle organization 1” (Apo1) protein from rice. This protein comprises an amino acid sequence selected from the group consisting of a) an amino acid sequence of SEQ ID NO: 3, 15 or 17 or a functional variant thereof, and b) an amino acid sequence having at least 85% sequence identity with the amino acid sequence of SEQ ID NO: 3, 15 or 17, or a functional variant thereof.

It is another object of the present invention to provide an isolated nucleic acid encoding the protein according to the invention, which may comprise a nucleotide sequence selected from the group consisting of a) a nucleic acid sequence of SEQ ID NO: 1 or SEQ ID NO: 2, b) a nucleic acid sequence having at least 80% identity to the nucleic acid sequence of SEQ ID NO: 1 or SEQ ID NO: 2, c) a nucleic acid having a complementary sequence to the nucleic acid of a) or b). The nucleic acid according to the invention may localize within an interval on wheat chromosome 7A comprising the nucleotide sequence comprised between the nucleotide at position 674,081,462 in the NRgene-HiC Chinese Spring reference genomic sequence and the nucleotide at position 674,082,918 in the NRgene-HiC Chinese Spring reference genomic sequence and flanked by markers of SEQ ID NO: 10 and SEQ ID NO: 11 or flanked by markers of SEQ ID NO:12 and either SEQ ID NO: 13 or SEQ ID NO: 14, or flanked by the markers of SEQ ID NO: 23 and SEQ ID NO: 24, or may localize within an interval on wheat chromosome 7B flanked by the markers of SEQ ID NO: 26 and 27. In one embodiment, an isolated nucleic acid encoding the protein according to the invention, may comprise a nucleotide sequence selected from the group consisting of a) a nucleic acid sequence of SEQ ID NO: 1, 2, 6, 7, 15, 16, 20, 21, 28, or 30, b) a nucleic acid sequence having at least 80% identity to the nucleic acid sequence of SEQ ID NO: 1, 2, 6, 7, 15, 16, 20, 21, 28, or 30, c) a nucleic acid having a complementary sequence to the nucleic acid of a) or b). In one embodiment, an isolated nucleic acid encoding the protein according to the invention, may comprise a nucleotide sequence selected from the group consisting of a) a nucleic acid sequence of SEQ ID NO: 1, 2, 6, 7, or 28, b) a nucleic acid sequence having at least 80% identity to the nucleic acid sequence of SEQ ID NO: 1, 2, 6, 7, or 28, c) a nucleic acid having a complementary sequence to the nucleic acid of a) or b). In one embodiment, any of such nucleic acid sequences is an isolated or artificial nucleic acid.

The present invention furthermore provides a recombinant gene comprising a plant expressible promoter operably linked to a nucleic acid sequence encoding the protein according to the invention and optionally, a transcription termination and polyadenylation sequence, preferably a transcription termination and polyadenylation region functional in plants. In another embodiment, the plant expressible promoter may be selected from a constitutive promoter, an inducible promoter or a tissue specific promoter. The plant expressible promoter may be a CaMV35S promoter, a Ubiquitin promoter or the native promoter of the APO1 gene according to the invention retrieved from a wheat variety with a relative high number of spikelets per spike.

In another aspect, the invention provides a wheat plant, plant part or seed consisting of wheat plant cells comprising the recombinant gene described herein.

In alternative embodiments, methods are provided for producing wheat plants with altered number of spikelets per spike or for altering the number of spikelets per spike of a wheat plant, both methods comprising the step of altering the abundance of the protein according to the invention within the wheat plant. In one embodiment, the abundance of the protein is increased and the number of spikelets per spike is increased compared to the number of spikelets per spike of the wheat plant wherein the abundance of the protein is not altered, particularly wherein the wheat plant has an initial low (relative) number of spikelets per spike. The abundance of the protein of the invention may be increased by providing said wheat plant with a) a recombinant gene according to the invention, or b) a heterologous gene encoding the protein according to the invention, wherein the heterologous gene is higher expressed than the corresponding endogenous gene. The heterologous gene may comprise the nucleotide sequence of SEQ ID NO: 4, 5, 9, 19 or SEQ ID NO: 22 or a nucleotide sequence having at least 90% sequence identity to any one of those sequences. In one embodiment, the heterologous gene may comprise the nucleotide sequence of SEQ ID NO: 4, 5, 9, 19, or a nucleotide sequence having at least 90% sequence identity to any one of those sequences, wherein said sequence is characterized by an about 115 nucleotide deletion (such as 100-130 nucleotides, or 115 nucleotides) at a position about 500 nucleotides upstream of the ATG start codon (corresponding to the start codon in the reference sequence of SEQ ID NO: 1).

In yet another embodiment, the abundance of the protein is decreased and the number of spikelets per spike is decreased compared to the number of spikelets per spike of the wheat plant where the abundance of the protein is not altered, particularly wherein the wheat plant has an initial high (relative) number of spikelets per spike. The abundance of the protein according to the invention may be decreased by providing the wheat plant with a) a heterologous gene encoding the protein according to the invention, wherein the promoter of said heterologous gene has a lower promoter activity than the promoter of the endogenous gene, or b) a mutant allele of the endogene encoding the protein according to the invention. The heterologous gene may comprise the nucleotide sequence of SEQ ID NO: 9 or a nucleotide sequence having at least 90% sequence identity thereto, and preferably not comprising the nucleotide sequence from position 4399 to position 4513 of SEQ ID NO: 5, or a nucleotide sequence having at least 90% sequence identity thereto. The heterologous gene may also comprise the nucleotide sequence of SEQ ID NO: 19 or a nucleotide sequence having at least 90% sequence identity thereto, preferably devoid of the nucleotide sequence from position 7816 to position 7930 of SEQ ID NO: 19, or a nucleotide sequence having at least 90% sequence identity thereto. The mutant allele may be a knock out allele. The mutant allele may also be a substitution mutant allele or deletion or insertion mutant allele preferably with lower activity.

In yet another embodiment, in the methods described above, the step of providing comprises providing by transformation, crossing, backcrossing, introgressing, genome editing or mutagenesis.

Further embodiments disclose methods for identifying and/or selecting a wheat plant comprising an allele of a gene contributing positively or negatively to the number of spikelets per spike, respectively comprising the step of identifying the presence or absence, respectively, in the genome of the wheat plant of a nucleic acid having the nucleotides from position 4399 to position 4513 of SEQ ID NO: 5, or of a nucleotide sequence having at least 90% sequence identity thereto or a nucleic acid having the nucleotide sequence from position 7816 to position 7930 of SEQ ID NO: 19, or of a nucleotide sequence having at least 90% sequence identity thereto.

### BRIEF DESCRIPTION OF THE FIGURES

FIG. 1: APO1 RNA expression level in different spring wheat varieties (MAGIC Founders) and contrasting HIFs with and without an allele contributing to SPS. TS: Terminal Spikelet, DR: Double Ridge. Baxter, Chara, Westonia and Yitpi are the parents of the 4-way MAGIC population. Fam1_A_1, Fam1_B_1, Fam2_B_1, Fam2_C_1, Fam2_H_1, Fam3_E_1, Fam3_I_1, Fam4_A, Fam4_G, Fam5_C_1 and Fam5_F_1 are eleven HIFs analysed. The lines having high a number of spikelets per spike are marked with an asterisk.

FIG. 2: A. Distribution of mean phenotypes of all lines from the 2014 winter wheat population phenotyped for total Spikelet Number per Spike (SPS) and indication of SPS for the founder wheat varieties. B. Summary of variation in SPS phenotypes and associated heritabilities.

FIG. 3: Finemapping of QTsn.jbl-7. a) Mpwgaim QTL model b) MAGIC genetic map alignment c) IWGSCv1 physical map with annotated MEGAP gene models d) Sequence polymorphisms between Robigus and Claire/Chinese Spring in an APO1 orthologue.

FIG. 4: Syntenic relationships of the QTsn.jbl-7A QTL to QTsn.jbl-7B QTL and the rice qPBN6 QTL.

FIG. 5: a) Expression of TaAPO1-7A transcript relative to the housekeeping genes TaRP15 [Shaw, L. M., A. S. Turner, and D. A. Laurie, Plant J, 2012. 71(1): p. 71-84] Ta2291 [Paolacci, A. R., et al., BMC Molecular Biology, 2009. 10(1): p. 11] and normalized to TaAPO1-7A expression in Brompton. b) Regression of expression of TaAPO1-7A on BLUP of Total Spikelet number for the MAGIC Founder lines in the 2014 field trial. All varieties were sampled at stage GS32 except Soissons which was at GS34 due to the accelerated flowering caused by the Ppd-D1 allele. The reasons for low TaAPO1-7A expression in Soissons is therefore likely different than that linked to the sequence variation observed in Robigus and Brompton.

### DETAILED DESCRIPTION

The present invention is based on the insight that the wheat ortholog of the rice Apo1 is involved in determining the number of spikelets per spike in wheat varieties, including spring and winter wheat varieties.

In one aspect, the invention provides a protein involved in determining the number of spikelets per spike in wheat which is orthologous to “aberrant panicle organization 1” (Apo1) from rice. This protein comprises an amino acid sequence selected from the group consisting of a) an amino acid sequence of SEQ ID NO: 3, 15 or 17 or a functional fragment thereof, and b) an amino acid sequence having at least 85% sequence identity with the amino acid sequence of SEQ ID NO: 3, 15 or 17, or a functional variant thereof.

The number of spikelets per spike is both genetically and environmentally controlled. Different wheat varieties have different average number of spikelets per spike in a given environment. The observed number of spikelets per spike on a primary stem varies between about 17 and about 40 depending on the observed wheat line. Spring wheat varieties, in general, have lower number of spikelets per spike (18-24) while winter wheat varieties typically have higher number of spikelets per spike. Where wheat lines contain a positively contributing allele of the SPS QTL, the number of spikelets is increased at least by 1, but sometimes 2 or 3 when compared to a similar line without the positively contributing allele, regardless of the remaining genetic make-up or the environment.

The term “protein” interchangeably used with the term “polypeptide” as used herein describes a group of molecules consisting of more than 30 amino acids, whereas the term “peptide” describes molecules consisting of up to 30 amino acids. Proteins and peptides may further form dimers, trimers and higher oligomers, i.e. consisting of more than one (poly)peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is effected e.g. by glycosylation, acetylation, phosphorylation and the like. Such modifications are well known in the art.

Ikeda et al. 2005 (Developmental Biology, 282:349-360) identified the ABERRANT PANICLE ORGANIZATION 1 (APO1) gene as a key floral regulator of rice. Loss of function of APO1 led to the precocious conversion of inflorescence meristems into spikelet meristems, resulting in a reduced number of spikelets (Ikeda et al 2005, Ikeda et al. 2007, Plant Journal 51, 1030-1040). Gain of function mutation in APO1 led to a delayed conversion of inflorescence meristems into spikelet meristems, resulting in an increased number of spikelets (Ikeda et al. 2007, Ikeda Kawakatsu et al. 2009, Plant physiol. 150:736-747). APO1 was furthermore identified by Terao et al. 2010 (Theor Appl Genet, 120:875-893) as the gene responsible for the quantitative trait locus positively controlling the number of primary rachis branches, the number of grains per panicle and the grain yield per rice plant.

A “gene orthologous to APO1” as used herein is a gene which is found in a different species but evolved from a common ancestral gene by speciation and retained the same function. APO1 encodes an F-box protein, and known orthologous genes include a gene from Arabidopsis named UNUSUAL FLORAL ORGANS (UFO) and a gene from petunia named DOUBLE TOP (DOT) which have also been shown to control the timing of the transition to flowering and the architecture of the inflorescence.

SEQ ID NO: 3 represents the amino acid sequence of the APO1 gene from the wheat variety Chinese Spring. The varieties Baxter and Westonia produce an APO1 protein having an amino acid sequence identical to the one of SEQ ID NO: 3. SEQ ID NO: 8 represents the amino acid sequence of the APO1 gene from the wheat variety Chara. The variety Yitpi produces an APO1 protein having an amino acid sequence identical to the one of SEQ ID NO: 8. An APO1 protein having the amino acid sequence of SEQ ID NO: 8 is a functional variant of the APO1 protein having the amino acid sequence of SEQ ID NO: 3. The variety Claire produces an APO1 protein having an amino acid sequence identical to the one of SEQ ID NO: 3. The varieties Robigus, Cadenza and Paragon produce an APO1 protein having an amino acid sequence of SEQ ID NO: 3, where the Phenylalanine at position 47 is substituted with a Cysteine and the Aspartic acid at position 384 is substituted with an Asparagine. An APO1 protein having the amino acid sequence of SEQ ID NO: 3, where the Phenylalanine at position 47 is substituted with a Cysteine and the Aspartic acid at position 384 is substituted with an Asparagine, is a functional variant of the APO1 protein having the amino acid sequence of SEQ ID NO: 3. SEQ ID NO: 29 represents the amino acid sequence of the APO1 gene on chromosome 7A from the wheat variety Chinese Spring according to an alternative gene model and lacks the 27 N-terminal amino acids of SEQ ID NO: 3. SEQ ID NO: 17 represents the amino acid sequence of the APO1 gene on chromosome 7B from the wheat varieties Chinese Spring and Claire. In Robigus, the protein is characterized by a H47R and A173S substitution. SEQ ID NO: 31 represents the amino acid sequence of the APO1 gene on chromosome 7B from the wheat variety Chinese Spring according to an alternative gene model and lacks the 71 N-terminal amino acids of SEQ ID NO: 17. SEQ ID NO: 3 shares 89% sequence identity with SEQ ID NO: 17. SEQ ID NOs: 29 and 31 share 98% sequence identity.

Suitable for the invention are APO1 proteins which comprise an amino acid sequence having at least 85%, or at least 90%, or at least 95%, or at least 98%, or at least 99% sequence identity or are identical to the herein described protein, also referred to as variants. The term “variant” with respect to the amino acid sequences SEQ ID NO: 3 or SEQ ID NO: 8 of the invention is intended to mean substantially similar sequences. In one embodiment, a variant of the protein of the invention is an artificial protein as defined, or is a variant protein that does not include any naturally-occurring protein.

As used herein, the term “percent sequence identity” refers to the percentage of identical amino acids between two segments of a window of optimally aligned amino acid sequences or to the percentage of identical nucleotides between two segments of a window of optimally aligned nucleotide sequences. Optimal alignment of sequences for aligning a comparison window are well-known to those skilled in the art and may be conducted by tools such as the local homology algorithm of Smith and Waterman (Waterman, M. S., Chapman & Hall. London, 1995), the homology alignment algorithm of Needleman and Wunsch (1970), the search for similarity method of Pearson and Lipman (1988), and preferably by computerized implementations of these algorithms such as GAP, BESTFIT, FASTA, and TFASTA available as part of the GCG (Registered Trade Mark), Wisconsin Package (Registered Trade Mark from Accelrys Inc., San Diego, Calif.). An “identity fraction” for aligned segments of a test sequence and a reference sequence is the number of identical components that are shared by the two aligned sequences divided by the total number of components in the reference sequence segment, i.e., the entire reference sequence or a smaller defined part of the reference sequence. Percent sequence identity is represented as the identity fraction times 100. The comparison of one or more amino acid or DNA sequences may be to a full-length amino acid or DNA sequence or a portion thereof, or to a longer amino acid or DNA sequence. Sequence identity is calculated based on the shorter nucleotide or amino acid sequence.

Furthermore, it is clear that variants of the wheat APO1 proteins, wherein one or more amino acid residues have been deleted, substituted or inserted, can also be used to the same effect in the methods according to the invention, provided that the F-box domain (SEQ ID NO: 3 from amino acid position 33 to amino acid position 77 (as defined in the Pfam database) is not affected by the deletion, substitution or insertion of amino-acid.

Examples of substitutions are the conservative substitutions, i.e. substitutions of one amino-acid by another having similar physiochemical properties. These substitutions are known not to affect the structure of a protein. Such substitutions are achieved by replacing one amino acid by another amino acid belonging to the same group as follows:
Group 1: Cysteine (C);
Group 2: Phenylalanine (F), Tryptophan (W) and Tyrosine (Y);
Group 3: Histidine (H), Lysing K) and Arginine (R);
Group 4: Aspartic acid (D), Glutamic acid (E), Asparagine (N) and Glutamine (Q);
Group 5: Isoleucine (I), Leucine (L), Methionine (M) and Valine (V);
Group 6: Alanine (A), Glycine (G), Proline (P), Serine (S) and Threonine (T).

It is another object of the present invention to provide a nucleic acid, including an isolated or artificial nucleic acid, encoding the protein according to the invention, which may comprise a nucleotide sequence selected from a) a nucleic acid sequence of any one of SEQ ID NO: 1, 2, 6, 7 or 28 , b) a nucleic acid sequence having at least 80% identity to the nucleic acid sequence of SEQ ID NO: 1, 2, 6, 7, or 28 and c) a nucleic acid having a complementary nucleotide sequence to the nucleic acid of a) or b). The nucleic acid according to the invention may localize within an interval on wheat chromosome 7A comprising the nucleotide sequence included between the nucleotide at position 674,081,462 and the nucleotide at position 674,082,918 of the Chinese Spring wheat reference genome (NRgene-HiC), and flanked by markers of SEQ ID NO: 10 and SEQ ID NO: 11 or flanked by markers of SEQ ID NO: 12 and either SEQ ID NO: 13 or SEQ ID NO: 14, or flanked by the markers of SEQ ID NO: 23 and SEQ ID NO: 24, or may localize within an interval on wheat chromosome 7B flanked by the markers of SEQ ID NO: 26 and 27.

“Isolated nucleic acid”, used interchangeably with “isolated DNA” as used herein refers to a nucleic acid not occurring in its natural genomic context, irrespective of its length and sequence. Isolated DNA can, for example, refer to DNA which is physically separated from the genomic context, such as a fragment of genomic DNA. Isolated DNA can also be an artificially produced DNA, such as a chemically synthesized DNA, or such as DNA produced via amplification reactions, such as polymerase chain reaction (PCR) well-known in the art. Isolated DNA can further refer to DNA present in a context of DNA in which it does not occur naturally. For example, isolated DNA can refer to a piece of DNA present in a plasmid. Further, the isolated DNA can refer to a piece of DNA present in another chromosomal context than the context in which it occurs naturally, such as for example at another position in the genome than the natural position, in the genome of another species than the species in which it occurs naturally, or in an artificial chromosome. An “artificial DNA”, or “artificial nucleic acid”, as used herein is a DNA or nucleic acid that differs from a naturally-occurring DNA or nucleic acid (either in sequence or in some other way, e.g., having one or more internal nucleotide deletions (excluding deletions at either end) that do not occur in nature, or nucleotide substitutions or insertions that do not occur in nature, having a different nucleotide sequence compared to the naturally-occurring sequence, being linked to a label or molecule to which the DNA or nucleic acid is not linked in nature (such as a linkage to a heterologous or artificial promoter or 3′ untranslated region), etc.). Similarly, an “artificial protein” of the invention is a protein that differs from a naturally-occurring protein (either in sequence or in any other way, e.g., having one or more amino acid deletions (in one embodiment these are internal amino acid deletions (not a deletion at either protein end)) not occurring in nature, or amino acid substitutions or insertions that do not occur in the protein in nature, having a different amino acid sequence compared to the naturally-occurring sequence, being linked to a label or molecule to which the protein is not linked in nature, etc.). The sequence of an artificial DNA or nucleic acid has been altered by man compared to the naturally-occurring form, such as by (chemical or other) mutagenesis, recombination, targeted genome or base editing using sequence-specific nucleases, and the like.

Suitable for the invention are nucleic acids, encoding a wheat APO1 protein, which comprise a nucleotide sequence having at least 40%, at least 50%, or at least 60%, or at least 70%, or at least 80%, or at least 85%, or at least 90%, or at least 95%, or at least 98% sequence identity to the herein described gene, and are also referred to as variants. The term “variant” with respect to any one of the nucleotide sequences SEQ ID NOs: 1, 2, 6, 7, or 28 of the invention is intended to mean substantially similar nucleotide sequences encoding amino acid sequences substantially similar to any one of the amino acid sequences of SEQ ID NO: 3, 8, or 29. The term “variant” with respect to any one of the nucleotide sequences SEQ ID Nos: 15, 20, 21 or 30 of the invention is intended to mean substantially similar nucleotide sequences encoding amino acid sequences substantially similar to any one of the amino acid sequences of SEQ ID No: 17 or 31. The term “variant” with respect to the nucleotide sequence of SEQ ID Nos: 16 of the invention is intended to mean substantially similar nucleotide sequences encoding amino acid sequences substantially similar to any one of the amino acid sequences of SEQ ID No: 18. Naturally occurring allelic variants can be identified with the use of well-known molecular biology techniques, as, for example, with polymerase chain reaction (PCR) and hybridization techniques as herein outlined. Variant nucleotide sequences also include synthetically derived nucleotide sequences, such as those generated, for example, by using site-directed mutagenesis of any one of SEQ ID NO: 1, 2, 6, 7, 15, 16, 20, 21, 28 or 30. Generally, nucleotide sequence variants of the invention will have at least 40%, 50%, 60%, to 70%, e.g., preferably 71%, 72%, 73%, 74%, 75%, 76%, 77%, 78%, to 79%, generally at least 80%, e.g., 81% to 84%, at least 85%, e.g., 86%, 87%, 88%, 89%, 90%, 91%, 92%, 93%, 94%, 95%, 96%, 97%, to 98% and 99% nucleotide sequence identity to any one SEQ ID NOs: 1, 2, 6, 7, 15, 16, 20, 21, 28 or 30. Derivatives of the DNA molecules disclosed herein may include, but are not limited to, deletions of sequence, single or multiple point mutations, alterations at a particular restriction enzyme site, addition of functional elements, or other means of molecular modification. Techniques for obtaining such derivatives are well-known in the art (see, for example, J. F. Sambrook, D. W. Russell, and N. Irwin (2000) Molecular Cloning: A Laboratory Manual, 3^{rd} edition Volumes 1, 2, and 3. Cold Spring Harbor Laboratory Press). Those of skill in the art are familiar with the standard resource materials that describe specific conditions and procedures for the construction, manipulation, and isolation of macromolecules (e.g., DNA molecules, plasmids, etc.), as well as the generation of recombinant organisms and the screening and isolation of DNA molecules. In one embodiment, a variant of the DNA or nucleic acid of the invention is an artificial DNA or nucleic acid, or is a variant DNA or nucleic acid that does not include any naturally-occurring DNA or nucleic acid.

SEQ ID NO: 1 represents the nucleotide sequence of the coding DNA of APO1 from the wheat variety Chinese Spring. SEQ ID NO: 2 represents the corresponding genomic DNA of APO1 from the variety Chinese Spring. SEQ ID NO: 28 represents the nucleotide sequence of the coding DNA of APO 1 on chromosome 7A from the wheat variety Chinese Spring according to an alternative gene model. The varieties Baxter and Westonia comprise an APO1 gene having a nucleotide sequence identical to SEQ ID NO:1 as the nucleotide sequence of the coding DNA, and a nucleotide sequence identical to SEQ ID NO: 2 for the corresponding genomic DNA of APO1. SEQ ID NO: 6 represents the nucleotide sequence of the coding DNA of APO1 from the wheat variety Chara. SEQ ID NO: 7 represents the corresponding genomic DNA of APO1 from the variety Chara. The variety Yitpi comprises an APO1 gene having a nucleotide sequence identical to SEQ ID NO: 6 as the nucleotide sequence of the coding DNA, and a nucleotide sequence identical to SEQ ID NO: 7. The variety Claire comprises an APO1 gene having as nucleotide sequence of the coding DNA and the corresponding genomic DNA of APO1 a sequence identical to the one of SEQ ID NO: 1 and SEQ ID NO: 2, respectively. The varieties Robigus, Cadenza and Paragon comprise an APO1 gene having as nucleotide sequence of the coding DNA the nucleotide sequence of SEQ ID NO: 1, where the Thymine at position 140 is substituted with a Guanine, the Guanine at position 1150 is substituted with an Alanine, and having as nucleotide sequence of the genomic DNA the nucleotide sequence of SEQ ID NO: 2 where the Thymine at position 140 is substituted with a Guanine, the Guanine at position 1284 is substituted with an Alanine. SEQ ID NO: 20 represents the nucleotide sequence of the coding DNA of APO1 from the wheat variety Chinese Spring on chromosome 7B. SEQ ID NO: 30 represents the nucleotide sequence of the coding DNA of APO 1 on chromosome 7B from the wheat variety Chinese Spring according to an alternative gene model. SEQ ID NO: 21 represents the corresponding genomic DNA of APO1-7B from the variety Chinese Spring. When looking at the key conserved SNPs and indels in the APO1 allele of Robigus (2 SNPs in the coding sequence (changing 2 amino acids), and 1 SNP in the intron) related to the SPS-phenotype, Brompton had the same conserved SNPs and indels as Robigus.

The Apo1 SPS-gene or allele of the invention (as in Robigus or Yitpi, e.g.) has the following key differences to the Chinese Spring reference Apo1 sequence, which differences are characteristic for all Apo1 SPS-alleles tested across different populations of spring or winter wheat. These characteristics differences to the Chinese Spring reference Apo1-7A sequence are selected from the group of: a) a 115 bp deletion about 500 nt upstream of ATG start codon, 2 missense SNPs (wherein a missense SNP is a single nucleotide change resulting in a codon that encodes a different amino acid) in the coding sequence, an about 5-7.5 kb deletion about 7.5 kp upstream of start codon, the SNPs and indels present in the about 5 kb promoter (such as the SNPs and indels shown in Table 2 below, for Yitpi/Chara), and a SNP in the intron, b) a 115 bp deletion about 500 nt upstream of ATG start codon, 2 missense SNPs in the coding sequence, an about 5-7.5 kb deletion about 7.5 kp upstream of start codon, the SNPs and indels present in the about 5 kb promoter (such as the SNPs and indels shown in Table 2 below, for Yitpi/Chara), c) a 115 bp deletion about 500 nt upstream of ATG start codon, 2 missense SNPs in the coding sequence, an about 5-7.5 kb deletion about 7.5 kp upstream of start codon, d) a 115 bp deletion about 500 nt upstream of ATG start codon, 2 missense SNPs in the coding sequence, or e) a 115 bp deletion about 500 nt upstream of ATG start codon. These differences conserved in the tested SPS-lines may contribute to the observed SPS phenotype. Of course, some other small differences (such as SNPs/indels) can occur between SPS-Apo1 alleles in different wheat plant backgrounds, but these are not believed to be biologically significant.

A nucleic acid comprising a nucleotide sequence having at least 80% sequence identity to SEQ ID NO: 1 or SEQ ID NO: 2 can thus be a nucleic acid comprising a nucleotide sequence having at least 80%, or at least 85%, or at least 90%, or at least 95%, or at least 98%, or at least 99% or 100% sequence identity to SEQ ID NO: 1 or SEQ ID NO: 2 respectively. The nucleotide sequence of SEQ ID NO: 6 has at least 99% sequence identity with the nucleotide sequence of SEQ ID NO: 1. The nucleotide sequence of SEQ ID NO: 7 has at least 99% sequence identity with the nucleotide sequence of SEQ ID NO: 2.

The present invention furthermore provides a recombinant gene comprising a plant expressible promoter, including a heterologous or artificial plant -expressible promoter, operably linked to an Apo1 nucleic acid sequence encoding an APO1 protein according to the invention and optionally, a transcription termination and polyadenylation sequence, preferably a transcription termination and polyadenylation region functional in plants. In one embodiment, the plant expressible promoter may be a constitutive promoter, inducible promoter or a tissue specific promoter. The plant expressible promoter may be the CaMV35S promoter, the Ubiquitin promoter or the native promoter of the Apo1 gene according to the invention retrieved from a wheat variety with a high number of spikelets per spike. In yet another embodiment the Apo1 nucleic acid is selected from a) a nucleic acid sequence of SEQ ID NO: 1, SEQ ID NO: 2, SEQ ID NO: 6, SEQ ID NO: 7, or SEQ ID NO: 28; or b) a nucleic acid sequence having at least 80% identity to the nucleic acid sequence of SEQ ID NO: 1, SEQ ID NO: 2, SEQ ID NO: 6, SEQ ID NO: 7, or SEQ ID NO: 28, or c) a nucleic acid having a complementary sequence to the nucleic acid of a) or b), such as an artificial nucleic acid.

As used herein, a “recombinant gene” is an artificial gene constructed by operably linking fragments of unrelated genes or other nucleic acid sequences. In other words, “recombinant gene” denotes a gene which is not normally found in a plant species or refers to any gene in which the promoter or one or more other regulatory regions of the gene are not associated in nature with a part or all of the transcribed nucleic acid, i.e. are heterologous with respect to the transcribed nucleic acid. More particularly, a recombinant gene is an artificial, i.e. non-naturally occurring, gene produced by operable linking a plant expressible promoter with a nucleic acid sequence encoding an APO1 protein.

As used herein, “plant-expressible promoter” means a region of DNA sequence that is essential for the initiation of transcription in a plant cell. This includes any promoter of plant origin, but also any promoter of non-plant origin which is capable of directing transcription in a plant cell, i.e. certain promoters of viral or bacterial origin such as such as the CaMV35S, the subterranean clover virus promoter No 4 or No 7 (WO9606932) or T-DNA gene promoters and the like.

Examples of constitutive promoters include promoters of bacterial origin, such as the octopine synthase (OCS) and nopaline synthase (NOS) promoters from Agrobacterium, but also promoters of viral origin, such as that of the cauliflower mosaic virus (CaMV) 35S transcript (Hapster et al., 1988, Mol. Gen. Genet. 212: 182-190) or 19S RNAs genes (Odell et al., 1985, Nature. 6; 313(6005):810-2; U.S. Pat. No. 5,352,605; WO 84/02913; Benfey et al., 1989, EMBO J. 8:2195-2202), the enhanced 2x35S promoter (Kay at al., 1987, Science 236:1299-1302; Datla et al. (1993), Plant Sci 94:139-149) promoters of the cassava vein mosaic virus (CsVMV; WO 97/48819, U.S. Pat. No. 7,053,205), 2xCsVMV (WO2004/053135) the circovirus (AU 689 311) promoter, the sugarcane bacilliform badnavirus (ScBV) promoter (Samac et al., 2004, Transgenic Res. 13(4):349-61), the figwort mosaic virus (FMV) promoter (Sanger et al., 1990, Plant Mol Biol. 14(3):433-43), the subterranean clover virus promoter No 4 or No 7 (WO 96/06932) and the enhanced 35S promoter as described in U.S. Pat. Nos. 5,164,316, 5,196,525, 5,322,938, 5,359,142 and 5,424,200. Among the promoters of plant origin, mention will be made of the promoters of the plant ribulose-biscarboxylase/oxygenase (Rubisco) small subunit promoter (U.S. Pat. No. 4,962,028; WO99/25842) from Zea mays and sunflower, the promoter of the Arabidopsis thaliana histone H4 gene (Chaboutéet aI., 1987), the Rice actin 1 promoter (Act-1, U.S. Pat. No. 5,641,876), the histone promoters as described in EP 0 507 698 A1, the Zea mays alcohol dehydrogenase 1 promoter (Adh-1) (from http://www.patentlens.net/daisy/promoters/242.html)). Also the small subunit promoter from Chrysanthemum may be used if that use is combined with the use of the respective terminator (Outchkourov et al., Planta, 216: 1003-1012, 2003). Particularly mentioned are the ubiquitin promoters (Holtorf et al., 1995, Plant Mol. Biol. 29:637-649, U.S. Pat. No. 5,510,474) of corn, rice and sugarcane, such as those described by Christensen and Quail (1996, Transgenic Research Vol 5 issue 3, pp 213-218).

Examples of inducible promoters include promoters regulated by application of chemical compounds, including alcohol-regulated promoters (see e.g. EP637339), tetracycline regulated promoters (see e.g. U.S. Pat. No. 5,464,758), steroid-regulated promoters (see e.g. U.S. Pat. Nos. 5,512,483; 6,063,985; 6,784,340; 6,379,945; WO01/62780), metal-regulated promoters (see e.g. U.S. Pat. No. 4,601,978).

Examples of tissue specific promoters include meristem specific promoters such as the rice OSH1 promoter (Sato et al. (1996) Proc. Natl. Acad. Sci. USA 93:8117-8122) rice metallothein promoter (BAD87835.1) WAK1 and WAK2 promoters (Wagner & Kohorn (2001) Plant Cell 13(2): 303-318, spike tissue specific promoter D5 from barley (US6291666), the lemma/palea specific Lem2 promoter from barley (Abebe et al. (2005) Planta, 221, 170-183), the early inflorescence specific Pvm1 promoter from barley (Alonse Peral et al. 2011, PLoS ONE 6(12) e29456), the early inflorescence specific Pcrs4/PrA2 promoter from barley (Koppolu et al. 2013, Proc. Natl. Acad. Sci USA, 110(32) 13198-13203), the meristem specific pkn1 promoter with the Act1 intron from rice (Zhang et al., 1998, Planta 204: 542-549, Postma-Haarsma et al. 2002, Plant Molecular Biology 48: 423-441) the SAM/inflorescence specific promoter from Dendrobium sp. Pdomads1 (Yu et al. 2002).

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence. “Functionally linked” is an equivalent term.

The term “heterologous” refers to the relationship between two or more nucleic acid or protein sequences that are derived from different sources. For example, a promoter is heterologous with respect to an operably linked nucleic acid sequence, such as a coding sequence, if such a combination is not normally found in nature. In addition, a particular sequence may be “heterologous” with respect to a cell or organism into which it is inserted (i.e. does not naturally occur in that particular cell or organism). For example, the recombinant gene disclosed herein is a heterologous nucleic acid.

Modulating the expression of the wheat APO1 gene, including increasing the expression thereof, leading to a modulated level of APO1 protein, including an increase of the APO1 protein, may also be achieved by providing the (wheat) plant with transcription factors that e.g. (specifically) recognize the APO1 promoter region and promote transcription, such as TALeffectors, dCas, dCpf1 etc coupled to transcriptional enhancers (see e.g. Moore et al. 2014 ACS Synth Biol. 3(10) 708-716; Qi et al. (2013) Cell 152(5) 1173-118, Liu et al. 2017 Nature Communications 8 Article Number 2095).

As used herein, the term “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the ones actually cited, i.e., they may be embedded in a larger nucleic acid or protein. A recombinant gene comprising a DNA region which is functionally or structurally defined may comprise additional DNA regions etc. However, in the context of the present disclosure, the term “comprising” also includes “consisting of”.

The recombinant genes as herein described optionally comprise a DNA region involved in transcription termination and polyadenylation. A variety of DNA regions involved in transcription termination and polyadenylation functional in plants are known in the art and those skilled in the art will be aware of terminator and polyadenylation sequences that may be suitable in performing the methods herein described. The polyadenylation region may be derived from a natural gene, from a variety of other plant genes, from T-DNA genes or even from plant viral genomes. The 3′ end sequence to be added may be derived from, for example, the nopaline synthase or octopine synthase genes, or alternatively from another plant gene, or from any other eukaryotic gene.

The phrases “DNA”, “DNA sequence,” “nucleic acid sequence,” “nucleic acid molecule” “nucleotide sequence” and “nucleic acid” refer to a physical structure comprising an orderly arrangement of nucleotides. The DNA sequence or nucleotide sequence may be contained within a larger nucleotide molecule, vector, or the like. In addition, the orderly arrangement of nucleic acids in these sequences may be depicted in the form of a sequence listing, figure, table, electronic medium, or the like.

In another aspect, the invention provides a wheat plant, plant part or seed consisting of wheat plant cells comprising the recombinant gene described herein.

“Wheat” or “wheat plant” as used herein can be any variety useful for growing wheat. Examples of wheat include, but are not limited to, Triticum aestivum, Triticum aethiopicum, Triticum compactum, Triticum dicoccoides, Triticum dicoccum, Triticum durum, Triticum monococcum, Triticum spelta, Triticum turgidum. “Wheat” furthermore encompasses spring and winter wheat varieties, with the winter wheat varieties being defined by a vernalization requirement to flower while the spring wheat varieties do not require such vernalization to flower.

“Plant parts” as used herein are parts of the plant, which can be cells, tissues or organs, such as seeds, severed parts such as roots, leaves, flowers, pollen, fibers etc.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents, such as seed obtained by selfing or crossing, e.g. hybrid seed (obtained by crossing two inbred parental lines), hybrid plants and plant parts derived there from are encompassed herein, unless otherwise indicated.

In some embodiments, the plant cells of the invention as well as plant cells generated according to the methods of the invention, may be non-propagating cells.

The plants obtained according to the invention can be used in a conventional breeding scheme to produce more plants with the same characteristics or to introduce the same characteristic into other varieties of the same or related plant species, or in hybrid plants. The plants obtained can further be used for creating propagating material. Plants according to the invention can further be used to produce gametes, seeds (including crushed seeds and seed cakes), seed oil, fibers, yarn, embryos, either zygotic or somatic, progeny or hybrids of plants obtained by methods of the invention. Seeds obtained from the plants according to the invention are also encompassed by the invention.

“Creating propagating material”, as used herein, relates to any means known in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

In some embodiments, methods are provided for producing wheat plant with an altered number of spikelets per spike or for altering the number of spikelets per spike of a wheat plant, both methods comprising the step of altering the abundance of the protein according to the invention within the wheat plant. In another embodiment, the abundance of the protein is increased and the number of spikelets per spike is increased compared to the number of spikelets per spike of the wheat plant where the abundance of the protein is not altered, particularly where the wheat plant has an initial low number of spikelets per spike. The abundance of the protein of the invention may be increased by providing said wheat plant with a) the recombinant or modified gene according to the invention, or b) a heterologous gene encoding the protein according to the invention, wherein the heterologous gene is higher expressed than the corresponding endogenous gene or c) as elsewhere described in this application through use of recombinant transcription effectors. The heterologous gene may comprise the nucleotide sequence of SEQ ID NO: 4, SEQ ID NO: 5, SEQ ID NO: 9, or SEQ ID NO: 19, or a nucleotide sequence having at least 90% sequence identity thereto.

In one embodiment, the abundance of the APO1-7A protein is increased, or the abundance of the APO1-7A protein and APO1-7B protein is increased, or the abundance of the APO1-7A protein and APO1-7D protein is increased, or the abundance of the APO1-7A, APO1-7B and APO1-7D proteins is increased.

In yet another embodiment, the abundance of the protein is decreased and the number of spikelets per spike is decreased compared to the number of spikelets per spike of the wheat plant where the abundance of the protein is not altered, particularly where the wheat plant has an initial high number of spikelets per spike. The abundance of the protein according to the invention may be decreased by providing the wheat plant with a) a heterologous gene encoding the protein according to the invention, wherein said heterologous gene is lower expressed than the corresponding endogenous gene, or b) a mutant allele of the endogene encoding the protein according to the invention. The heterologous gene may comprise the nucleotide sequence of SEQ ID NO: 9 or a nucleotide sequence having at least 90% sequence identity thereto, and preferably is devoid of the nucleotide sequence from position 4399 to position 4513 of SEQ ID NO: 4 or, SEQ ID NO: 5, or is devoid of the nucleotide sequence from position 7816 to position 7930 in SEQ ID NO: 19, or a nucleotide sequence having at least 90% sequence identity thereto. The mutant allele may be a knock out allele or a substitution allele with lower activity than the wild type allele. In one embodiment, the abundance of the APO1-7A protein is decreased, or the abundance of the APO1-7A protein and APO1-7B protein is decreased, or the abundance of the APO1-7A protein and APO1-7D protein is decreased, or the abundance of the APO1-7A, APO1-7B and APO1-7D proteins is decreased.

A wheat plant having an initial low number of spikelets per spike means a wheat plant from a variety which has an average number of spikelets per spike of less than about 23, less than about 22, less than about 21, less than about 20, less than about 19, or less than about 18 spikelets per spike. Said variety may have an average number of spikelets per spike between about 17 and about 23, between about 17 and about 22, between about 17 and about 21, between about 17 and about 20, between about 17 and about 19, between about 17 and about 18, between about 18 and about 23, between about 18 and about 22, between about 18 and about 21, between about 18 and about 20, between about 18 and about 19, between about 19 and about 23, between about 19 and about 22, between about 19 and about 21, between about 19 and about 20, between about 20 and about 23, between about 20 and about 22, between about 20 and about 21, between about 21 and about 23, between about 21 and about 22, between about 22 and about 23 spikelets per spike.

A wheat plant having an initial high number of spikelets per spike means a wheat plant from a variety which has an average number of spikelets per spike of at least about 23, at least about 24, at least about 25, or at least about 26, at least about 27, at least about 28, or at least about 29 or at least about 30 spikelets per spike. Said variety may have an average number of spikelets per spike between about 23 and about 30, between about 24 and about 30, between about 25 and about 30, between about 26 and about 30, between about 27 and about 30, between about 28 and about 30, between about 29 and about 30, between about 23 and about 29, between about 24 and about 29, between about 25 and about 29, between about 26 and about 29, between about 27 and about 29, between about 28 and about 29, between about 23 and about 28, between about 24 and about 28, between about 25 and about 28, between about 26 and about 28, between about 27 and about 28, between about 23 and about 27, between about 24 and about 27, between about 25 and about 27, between about 26 and about 27, between about 23 and about 26, between about 24 and about 26, between about 25 and about 26, between about 23 and about 25, between about 24 and about 25, or between about 23 and about 24 spikelets per spike.

“Altering the number of spikelets per spike” as used herein means to significantly increase or significantly decrease the average number of spikelets per spike of a wheat plant.

An increase of the number of spikelets per spike refers to an increase of at least about 1, at least about 2, at least about 3, at least about 5 spikelets per spike compared to the number of spikelets per spike of the wheat plant, particularly a wheat plant having an initial low number of spikelets per spike.

A decrease of the number of spikelets per spike refers to a decrease of at least about 3, at least 2, or at least 1 spikelets per spike compared to the number of spikelets per spike of the wheat plant, particularly in a wheat plant having an initial high number of spikelets per spike.

“Altering the abundance of the protein” as used herein means to (significantly) increase or (significantly) decrease the abundance of the protein described herein.

An increase refers to an increase by at least 10% at least 20%, at least 30%, at least 40%, 50%, at least 60%, at least 70%, at least 80%, at least 90%, at least 100% as compared to the amount of the protein produced by the cell of the wheat plant, particularly a wheat plant having initial low number of spikelets per spike.

A decrease refers to a decrease by at least 10%, at least 20%, at least 25%, at least 30%, at least 35%, at least 40%, at least 45% or at least 50% as compared to the amount of the protein produced by the cell of the wheat plant, particularly a wheat plant having initial high number of spikelets per spike.

In one embodiment, decreasing the expression and/or activity of the APO1 gene and/or protein can be by decreasing the amount of functional APO1 protein produced. Said decrease can be a decrease with at least 30%, 40%, 50%, 60%, 70%, 80%, 90%, 95% or 100% (i.e., no functional APO1 protein is produced by the cell) as compared to the amount of functional APO1 protein produced by a cell with wild type APO1 expression levels and activity. Said decrease in expression and/or activity can be a constitutive decrease in the amount of functional APO1 protein produced. Said decrease can also be a temporal/inducible decrease in the amount of functional APO1 protein produced.

Decreased expression and/or activity of the APO1 gene of the invention can also be achieved by using an RNA molecule that results in decreased expression and/or activity of the APO1 gene. An RNA molecule that results in a decreased expression and/or activity of an APO1 gene and/or protein can be an RNA encoding a protein which inhibits expression and/or activity of said APO1 protein. Further, said RNA molecule that results in a decreased expression and/or activity of an APO1 gene and/or protein can also be an RNA molecule which inhibits expression of a gene which is an activator of expression and/or activity of said APO1 protein. Said RNA molecule that inhibits the expression and/or activity of an APO1 gene and/or protein may also be an RNA molecule that directly inhibits expression and/or activity of an APO1 gene and/or protein, such as an RNA which mediates silencing of said APO1 gene.

The expression and/or activity of the APO1 gene and/or protein can conveniently be reduced or eliminated by transcriptional or post-transcriptional silencing of the expression of endogenous APO1 genes. To this end, a silencing RNA molecule can be introduced in the plant cells targeting the endogenous APO1 encoding genes. As used herein, “silencing RNA” or “silencing RNA molecule” refers to any RNA molecule, which upon introduction into a plant cell, reduces the expression of a target gene.

Silencing RNA may also be artificial micro-RNA molecules as described e.g. in WO2005/052170, WO2005/047505 or US 2005/0144667, or ta-siRNAs as described in WO2006/074400 (all documents incorporated herein by reference). In some embodiments, the nucleic acid expressed by the chimeric gene of the invention is catalytic RNA or has ribozyme activity specific for the target sequence. Thus, the polynucleotide causes the degradation of the endogenous messenger RNA transcribed from the target gene/sequence, resulting in reduced expression of the protein present in the plant. In one embodiment, the nucleic acid expressed by the chimeric gene of the invention encodes a zinc finger protein that binds to the gene encoding said protein, resulting in reduced expression of the target gene. In particular embodiments, the zinc finger protein binds to a regulatory region of said gene. In other embodiments, the zinc finger protein binds to a messenger RNA encoding said protein, thereby preventing its translation.

In alternative embodiments, decreasing the expression and/or activity of an APO1 gene and/or protein can be achieved by inhibition of the expression said APO1 protein present in the plant. Inhibition of the expression of said APO1 gene and/or protein can be induced at the desired moment using a spray (systemic application) with inhibitory nucleic acids, such as RNA or DNA molecules that function in RNA-mediated gene silencing, as e.g. described in WO2011/112570 (incorporated herein by reference).

In one embodiment of the invention, a yield increase can be obtained when wheat plants having a lower number of spikelets per spike (the SPS− allelic form of APO1-7A), are grown in certain environments, but the same plants when grown in another environment, can show a yield increase when having a higher number of spikelets per spike (the SPS+ allelic form of APO1). Whilst the yield effects can hence be reversed in different growing environments, the effects for SPS are consistent across environments. Such rank changes across environments (for yield in this case) is referred to as Genotype by Environment (G×E) interaction and is a major constraint on genetic gain in crops. By identifying the underlying gene it is possible to exploit the appropriate allele for each target environment.

SEQ ID NO: 4 represents the nucleotide sequence of the about 5 kb non coding DNA 5′ upstream of APO1 from the wheat variety Westonia. SEQ ID NO: 5 represents the nucleotide sequence of the about 5 kb non coding DNA 5′ upstream of APO1 from the wheat variety Baxter. SEQ ID NO: 4 and SEQ ID NO: 5 are functional variants and share 99% sequence identity. SEQ ID NO: 9 represents the nucleotide sequence of the corresponding non coding DNA 5′ upstream of APO1 from the wheat variety Chara. The variety Yitpi comprise a corresponding non coding DNA 5′ upstream of APO1 having a nucleotide sequence identical to SEQ ID NO: 9. SEQ ID NO: 19 represents the nucleotide sequence of the about 8kb non coding DNA 5′ upstream of APO1 from the wheat variety Chinese Spring on chromosome 7A. The variety Robigus comprises a corresponding non coding DNA 5′ upstream of APO1 having a nucleotide sequence of SEQ ID NO: 19, with a deletion of the nucleotides from position 7816 to 7930 of SEQ ID NO: 19 and an insertion of about 5-7.7 Kb nucleotides at nucleotide position 901 on SEQ ID NO: 19 (more specifically, between nucleotide position 900 and nucleotide position 901 of SEQ ID NO: 19—see first misc_feature in SEQ ID NO: 19). In addition Robigus has the same SNPs and indels as varieties Yitpi/Chara in Table 2, while Claire has the same SNPs and indels as Westonia in Table 2.

A nucleic acid comprising a nucleotide sequence having at least 90% sequence identity to SEQ ID NO: 4, 5, 9, or 19, can thus be a nucleic acid comprising a nucleotide sequence having at least 90%, or at least 95%, or at least 98%, or at least 99% or 100% sequence identity to SEQ ID NO: 4, 5, 9, or 19 respectively. A nucleotide sequence having 100% sequence identity to SEQ ID NO: 4, 5 or 9, is also referred to a nucleotide sequence being identical to SEQ ID NO: 4, 5 or 9, respectively. The nucleotide sequence of SEQ ID NO: 9 has 97% identity with the nucleotide sequence of SEQ ID NO: 4 or 5 but does not comprise the nucleotide sequence from position 4399 to position 4513 of SEQ ID NO: 5, or the nucleotide sequence from position 4401 to position 4516 of SEQ ID NO: 4.

In yet another embodiment, in the methods described above the “step of providing” may mean providing by transformation, crossing, backcrossing, introgressing, genome editing or mutagenesis.

The term “providing” may refer to introduction of an exogenous DNA molecule to a plant cell by transformation, optionally followed by regeneration of a plant from the transformed plant cell. The term may also refer to introduction of the recombinant DNA molecule by crossing of a transgenic plant comprising the recombinant DNA molecule with another plant and selecting progeny plants which have inherited the recombinant DNA molecule or transgene. Yet another alternative meaning of providing refers to introduction of the recombinant DNA molecule by techniques such as protoplast fusion, optionally followed by regeneration of a plant from the fused protoplasts.

It will be clear that the methods of transformation used are of minor relevance to the current invention. Transformation of plants is now a routine technique. Advantageously, any of several transformation methods may be used to introduce the nucleic acid/gene of interest into a suitable ancestor cell. Transformation methods include the use of liposomes, electroporation, chemicals that increase free DNA uptake, injection of the DNA directly into the plant(cell) such as microinjection, particle gun bombardment, transformation using viruses or pollen and microprojection. Methods may be selected from the calcium/polyethylene glycol method for protoplasts (Krens et al. (1982) Nature 296: 72-74; Negrutiu et al. (1987) Plant. Mol. Biol. 8: 363-373); electroporation of protoplasts (Shillito et al. (1985) Bio/Technol. 3: 1099-1102); microinjection into plant material (Crossway et al. (1986) Mol. Gen. Genet. 202: 179-185); DNA or RNA-coated particle bombardment (Klein et al. (1987) Nature 327: 70) infection with (non-integrative) viruses and the like.

Methods to transform wheat plants are also well known in the art. Different transformation systems could be established for various cereals: the electroporation of tissue, the transformation of protoplasts and the DNA transfer by particle bombardment in regenerable tissue and cells (for an overview see Jane, Euphytica 85 (1995), 35-44). The transformation of wheat has been described several times in literature (for an overview see Maheshwari, Critical Reviews in Plant Science 14 (2) (1995), 149-178, Nehra et al., Plant J. 5 (1994), 285-297). An efficient Agrobacterium-mediated transformation method has been described by Ishida et al. 2015 Agrobacterium protocols: Volume 1, Methods in Molecular Biology, vol. 1223 : 189-198.

“Mutagenesis”, as used herein, refers to the process in which plant cells (e.g., a plurality of wheat seeds or other parts) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), T-DNA insertion mutagenesis (Azpiroz-Leehan et al. (1997) Trends Genet 13:152-156), transposon mutagenesis (McKenzie et al. (2002) Theor Appl Genet 105:23-33), or tissue culture mutagenesis (induction of somaclonal variations), or a combination of two or more of these. Thus, the desired mutagenesis of one or more APO1 alleles may be accomplished by use of one of the above methods. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, wheat plants are regenerated from the treated cells using known techniques. For instance, the resulting wheat seeds may be planted in accordance with conventional growing procedures and following self-pollination seed is formed on the plants. Additional seed that is formed as a result of such self-pollination in the present or a subsequent generation may be harvested and screened for the presence of mutant apo1 alleles. Several techniques are known to screen for specific mutant alleles, e.g., Deleteagene™ (Delete-a-gene; Li et al., 2001, Plant J 27: 235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc.

The term “gene targeting” refers herein to directed gene modification that uses mechanisms such as homologous recombination, mismatch repair or site-directed mutagenesis. The method can be used to replace, insert and delete endogenous sequences or sequences present or previously introduced in plant cells. Methods for gene targeting can be found in, for example, WO 2006/105946 or WO2009/002150. Gene targeting can be used to create mutant or artificial apo1 alleles.

Gene targeting can also be used to create novel haplotypes or haplotype blocks. E.g. haplotype blocks comprising an APO1 gene on chromosome 7A, which may be beneficial for the yield potential in several ways, but comprise the upstream deletion and/or insertion associated with low SPS numbers, may be engineered through gene targeting to replace the upstream deletion and/or insertion.

“Wild type” (also written “wildtype” or “wild-type”), as used herein, refers to a typical form of a plant or a gene as it most commonly occurs in nature. A “wild type plant” refers to a plant with the most common phenotype of such plant in the natural population. A “wild type allele” refers to an allele of a gene required to produce the wild-type phenotype. By contrast, a “mutant plant” refers to a plant with a different rare phenotype of such plant produced by human intervention, e.g. by mutagenesis, and a “mutant allele” refers to an allele of a gene required to produce the mutant phenotype.

“Mutant” as used herein refers to a form of a plant or a gene which is different from such plant or gene in the natural population, and which is produced by human intervention, e.g. by mutagenesis, and a “mutant allele” refers to an allele which is not found in plants in the natural population or breeding population, but which is produced by human intervention such as mutagenesis or gene targeting.

As used herein, the term “wild type allele” (e.g. wild type APO1 allele), means a naturally occurring allele found within plants, in particular wheat plants, which encodes a functional protein (e.g. a functional APO1 protein). In contrast, the term “mutant allele” (e.g. mutant apo1 allele), as used herein, refers to an allele, which does not encode a functional protein, i.e. an apo1 allele encoding a non-functional APO1 protein, which, as used herein, refers to an APO1 protein having no biological activity or a significantly reduced biological activity as compared to the corresponding wild-type functional APO1 protein, or encoding no APO1 protein at all.

A “full knock-out” or “null” mutant allele, as used herein, refers to a mutant allele, which encodes a protein having no biological activity as compared to the corresponding wild-type functional protein or which encodes no protein at all. Such a “full knock-out mutant allele” is, for example, a wild-type allele, which comprises one or more mutations in its nucleic acid sequence, for example, one or more non-sense or mis-sense mutations. In particular, such a full knock-out mutant apo1 allele is a wild-type APO1 allele, which comprises a mutation that preferably result in the production of an APO1 protein lacking at least one functional domain, such as the F-box domain, or lacking at least one amino acid critical for its function, such that the biological activity of the APO1 protein is completely abolished, or whereby the mutation(s) preferably result in no production of an APO1 protein.

A “partial knock-out” mutant allele, as used herein, refers to a mutant allele, which encodes a protein having a significantly reduced biological activity as compared to the corresponding wild-type functional protein. Such a “partial knock-out mutant allele” is, for example, a wild-type allele, which comprises one or more mutations in its nucleic acid sequence, for example, one or more mis-sense mutations. In particular, such a partial knockout mutant allele is a wild-type allele, which comprises a mutation that preferably result in the production of an protein wherein at least one conserved and/or functional amino acid is substituted for another amino acid, such that the biological activity is significantly reduced but not completely abolished.

The expression level of a gene may be determined by those skilled in the art, for example using analysis of RNA accumulation produced from the nucleic acid. The RNA accumulation, or levels of RNA, such as mRNA, can be measured either at a single time point or at multiple time points, in a single tissue or in several tissues, and as such the fold increase can be average fold increase or an extrapolated value derived from experimentally measured values. The expression level may be determined by techniques such RT-qPCR, or by using hybridization based microarrays. The expression level may also be estimated by whole transcriptome shotgun sequencing, using next-generation sequencing to reveal the presence and quantity of RNA, which may be selected for polyadenylated RNA, or depleted of ribosomal RNA.

In certain embodiments, the step of modifying an endogenous Apo1 gene may comprise performing nucleotide modifications in an endogenous Apo1 gene in order to increase or decrease SPS in a plant.

In certain embodiments of the plants or methods as taught herein, the endogenous Apo1 gene may be modified by genome editing. In certain embodiments, genome editing may be performed with one or more engineered nucleases selected from the group consisting of RNA-guided nucleases, meganucleases, zinc finger nucleases (ZFNs), and transcription activator-like effector-based nucleases (TALEN).

In certain embodiments, the step of providing the plant may comprise: providing a wild type plant; and modifying an endogenous Apo1 gene in the plant by genome editing to obtain a plant comprising a nucleic acid as defined herein.

The term “genome editing” or “genome editing with engineered nucleases” generally refer to a type of genetic engineering in which DNA is inserted, deleted or replaced in the genome of a living organism using (engineered) nucleases. The nucleases create site-specific breaks, such as double-strand breaks (DSBs) at desired locations in the genome.

In certain embodiments, the endogenous Apo1 gene may be modified by creating site-specific breaks, such as double-strand breaks (DSBs), at one or more desired locations in the genome. The induced double-strand breaks may be repaired through non-homologous end joining (NHEJ) or homology directed repair (HDR).

In certain embodiments, the endogenous Apo1 gene may be modified by a method for genome editing, i.e., a method for modifying the genome, preferably the nuclear genome, of a plant cell at a preselected site, the method comprising the steps of:
inducing a double stranded DNA break (DSB) in the genome of said cell at a cleavage site at or near a recognition site for a double stranded DNA break inducing (DSBI) enzyme by expressing in said cell a DSBI enzyme recognizing said recognition site and inducing said DSB at said cleavage site;
introducing into said cell a repair nucleic acid molecule comprising an upstream flanking region having homology to the DNA region upstream of said preselected site and/or a downstream flanking DNA region having homology to the DNA region downstream of said preselected site for allowing homologous recombination between said flanking region or regions and said DNA region or regions flanking said preselected site; and
selecting a cell wherein said repair nucleic acid molecule has been used as a template for making a modification of said genome at said preselected site.
wherein said modification is selected from a replacement of at least one nucleotide, a deletion of at least one nucleotide, an insertion of at least one nucleotide, or any combination thereof.

As used herein, a “double stranded DNA break inducing enzyme” is an enzyme capable of inducing a double stranded DNA break at a particular nucleotide sequence, called the “recognition site”.

Rare-cleaving endonucleases are DSBI enzymes that have a recognition site of about 14 to 70 consecutive nucleotides, and therefore have a very low frequency of cleaving, even in larger genomes such as most plant genomes. Homing endonucleases, also called meganucleases, constitute a family of such rare-cleaving endonucleases. They may be encoded by introns, independent genes or intervening sequences, and present striking structural and functional properties that distinguish them from the more classical restriction enzymes, usually from bacterial restriction-modification Type II systems. Their recognition sites have a general asymmetry which contrast to the characteristic dyad symmetry of most restriction enzyme recognition sites. Several homing endonucleases encoded by introns or inteins have been shown to promote the homing of their respective genetic elements into allelic intronless or inteinless sites. By making a site-specific double strand break in the intronless or inteinless alleles, these nucleases create recombinogenic ends, which engage in a gene conversion process that duplicates the coding sequence and leads to the insertion of an intron or an intervening sequence at the DNA level.

A list of other rare cleaving meganucleases and their respective recognition sites is provided in Table I of WO03/004659 (pages 17 to 20) (incorporated herein by reference). These include I-Sce I, I-Chu I, I-Dmo I, I-Cre I, I-Csm I, PI-Fli I, Pt-Mtu I, I-Ceu I, I-Sce II, I-Sce III, HO, PI-Civ I, PI-Ctr I, PI-Aae I, PI-BSU I, PI-DhaI, PI-Dra I, PI-May I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tho I or PI-Tsp I.

Furthermore, methods are available to design custom-tailored rare-cleaving endonucleases that recognize basically any target nucleotide sequence of choice. Briefly, chimeric restriction enzymes can be prepared using hybrids between a zinc-finger domain designed to recognize a specific nucleotide sequence and the non-specific DNA-cleavage domain from a natural restriction enzyme, such as FokI. Such methods have been described e.g. in WO 03/080809, WO94/18313 or WO95/09233 and in Isalan et al., 2001, Nature Biotechnology 19, 656-660; Liu et al. 1997, Proc. Natl. Acad. Sci. USA, 94, 5525-5530).

Custom-made meganucleases can be produced by selection from a library of variants, is described in WO2004/067736. Custom made meganucleases with altered sequence specificity and DNA-binding affinity may also be obtained through rational design as described in WO2007/047859.

Another example of custom-designed endonucleases include the so-called TALE nucleases (TALENs), which are based on transcription activator-like effectors (TALEs) from the bacterial genus Xanthomonas fused to the catalytic domain of a nuclease (e.g. FOKI). The DNA binding specificity of these TALEs is defined by repeat-variable diresidues (RVDs) of tandem-arranged 34/35-amino acid repeat units, such that one RVD specifically recognizes one nucleotide in the target DNA. The repeat units can be assembled to recognize basically any target sequences and fused to a catalytic domain of a nuclease create sequence specific endonucleases (see e.g. Boch et al., 2009, Science, 326:p 1509-1512; Moscou and Bogdanove, 2009, Science, 326:p 1501; Christian et al., 2010, Genetics, 186:p 757-761; and WO10/079430, WO11/072246, WO2011/154393, WO11/146121, WO2012/001527, WO2012/093833, WO2012/104729, WO2012/138927, WO2012/138939). WO2012/138927 further describes monomeric (compact) TALENs and TALENs with various catalytic domains and combinations thereof.

Another customizable endonuclease system has been described; the so-called Clustered Regularly Interspaced Short Palindromic Repeats (CRISPR)/Cas system, which employs a special RNA molecule (crRNA) conferring sequence specificity to guide the cleavage of an associated RNA-guided endonuclease. Such custom designed rare-cleaving endonucleases are also referred to as non-naturally occurring rare-cleaving endonucleases.

An RNA-guided nuclease or RNA-guided endonuclease (RGEN), as used herein, is an RNA-guided DNA modifying polypeptide having (endo)nuclease activity.

RGENs are typically derived from the Clustered Regularly Interspaced Short Palindromic Repeats (CRISPR) systems, which are a widespread class of bacterial systems for defense against foreign nucleic acid. CRISPR systems are found in a wide range of eubacterial and archaeal organisms. CRISPR systems include type I, II, III and V sub-types (see e.g. WO2007025097; WO2013098244; WO2014022702; WO2014093479; WO2015155686; EP3009511; US2016208243). Wild-type type II CRISPR/Cas systems utilize an RNA-guided nuclease, e.g. Cas9, in complex with guide and activating RNA to recognize and cleave foreign nucleic acid (Jinek et al., 2012, Science, 337(6096):816-21).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Further Cas9 proteins, homologs and variants thereof and methods for use in genome editing or are described in, e.g., Chylinksi, et al., 2013, RNA Biol., 10(5): 726-737; Makarova et al., 2011, Nat. Rev. Microbiol., 9(6): 467-477; Hou, et al., 2013, Proc Natl Acad Sci USA, 110(39):15644-9; Sampson et al., 2013, Nature, 497(7448):254-7; Jinek, et al., 2012, supra; WO2013142578; WO2013176772; WO2014065596; WO2014089290; WO2014093709; WO2014093622; WO2014093655; WO2014093701; WO2014093712; WO2014093635; WO2014093595; WO2014093694; WO2014093661; WO2014093718; WO2014093709; WO2014099750; WO2014113493; WO2014190181; WO2015006294; WO2015071474; WO2015077318; WO2015089406; WO2015103153; WO201621973; WO201633298; WO201649258, all incorporated herein by reference.

Further RNA-guided nucleases include e.g. Cpf1 and homologues and variants thereof (as e.g. described in Zetsche et al., 2015, Cell, Volume 163, Issue 3, 759-771; EP3009511; US2016208243; Kleinstiver et al., 2016, Nat Biotechnol., 34(8):869-74; Gao et al., 2016, Cell Res., 6(8):901-13; Hur et al., 2016, Nat Biotechnol., 34(8):807; Kim et al., 2016, Nat Biotechnol., 34(8):863-8.; Yamano et al., 2016, Cell, 165(4):949-62), and also C2c1 and C2c3 (Shmakov et al., 2015, Mol Cell., 60(3):385-97), all incorporated herein by reference.

Further RNA-guided nucleases can include Argonaut-like proteins, for instance as described in WO2015157534.

Further RNA-guided nucleases and other polypeptides are described in WO2013088446.

In one embodiment, the RGEN can also be an RNA-guided nicking enzyme (nickase), or a pair of RNA-guided nicking enzymes, that each introduces a break in only one strand of the double stranded DNA at or near the preselected site. Of a pair of nickases, the one enzyme introduces a break in one strand of the DNA at or near the preselected site, while the other enzyme introduces a break in the other strand of the DNA at or near the preselected site. The two single-stranded breaks can be introduced at the same nucleotide position on both strands, resulting in a blunt ended double stranded DNA break, but the two single stranded breaks can also be introduced at different nucleotide positions in each strand, resulting in a 5′ or 3′ overhang at the break site (“sticky ends” or “staggered cut”). Nicking mutants and uses thereof are e.g. described in the above documents and specifically in WO2014191518, WO2014204725, and WO201628682. Also a single nicking mutant, which introduced a break in only one of the two strands of the DNA (i.e. a single-stranded DNA break), can enhance homology directed repair (HDR) with a donor polynucleotide (Richardson et al. 2016, Nature Biotechnology 34, 339-344; US62/262,189).

As an alternative to a nuclease or nickase, also nuclease deficient (also referred to as “dead” or catalytically inactive) variants of the above described nucleases, such as dCas9, can be used to increase targeted insertion of a donor polynucleotide, as e.g. described in Richardson et al. 2016, Nature Biotechnology 34, 339-344; US62/262,189). Such variants lack the ability to cleave or nick DNA but are capable of being targeted to and bind DNA (see e.g. WO2013176772, EP3009511). These “dead” nucleases are believed to induce strand displacement by binding to one of the two strands (“DNA melting”), thereby enhancing recombination with the donor polynucleotide by allowing the donor polynucleotide to anneal with the other “free” DNA strand.

Nicking mutants have been described of various RGENs and involve one or more mutations in a catalytic domain, such as the HNH and RuvC domains (e.g. Cas9) of the RuvC-like domain (e.g. Cpf1). For example, SpCas9 can be converted into a nickase by mutating DlOA in the RuvC and 863A in the HNH nuclease domain converts SpCas9 into a DNA nickase, while inactivation of both nuclease domain results in a catalytically inactive protein (Jinek et al., 2012, supra, Gasiunas et al., 2012, Proc. Natl. Acad. Sci. USA 109, E2579-E2586). In Cpf1, it was found that the D917A as well as the E1006A mutation completely inactivated the DNA cleavage activity of FnCpf1, and while D1255A significantly reduced nucleolytic activity (Zetsche et al., 2015, supra). Corresponding residues of other RGEN (e.g. Cas9 or Cpf1) variants can be determined by optimal alignment.

The cleavage site of a DSBI enzyme relates to the exact location on the DNA where the double-stranded DNA break is induced. The cleavage site may or may not be comprised in (overlap with) the recognition site of the DSBI enzyme and hence it is said that the cleavage site of a DSBI enzyme is located at or near its recognition site. The recognition site of a DSBI enzyme, also sometimes referred to as binding site, is the nucleotide sequence that is (specifically) recognized by the DSBI enzyme and determines its binding specificity. For example, a TALEN or ZNF monomer has a recognition site that is determined by their RVD repeats or ZF repeats respectively, whereas its cleavage site is determined by its nuclease domain (e.g. FOKI) and is usually located outside the recognition site. In case of dimeric TALENs or ZFNs, the cleavage site is located between the two recognition/binding sites of the respective monomers, this intervening DNA region where cleavage occurs being referred to as the spacer region. For meganucleases on the other hand, DNA cleavage is effected within its specific binding region and hence the binding site and cleavage site overlap.

A person skilled in the art would be able to either choose a DSBI enzyme recognizing a certain recognition site and inducing a DSB at a cleavage site at or in the vicinity of the preselected site or engineer such a DSBI enzyme. Alternatively, a DSBI enzyme recognition site may be introduced into the target genome using any conventional transformation method or by crossing with an organism having a DSBI enzyme recognition site in its genome, and any desired DNA may afterwards be introduced at or in the vicinity of the cleavage site of that DSBI enzyme.

As used herein, a repair nucleic acid molecule, is a single-stranded or double-stranded DNA molecule or RNA molecule that is used as a template for modification of the genomic DNA at the preselected site in the vicinity of or at the cleavage site. As used herein, use as a template for modification of the genomic DNA, means that the repair nucleic acid molecule is copied or integrated at the preselected site by homologous recombination between the flanking region(s) and the corresponding homology region(s) in the target genome flanking the preselected site, optionally in combination with non-homologous end-joining (NHEJ) at one of the two end of the repair nucleic acid molecule (e.g. in case there is only one flanking region). Integration by homologous recombination will allow precise joining of the repair nucleic acid molecule to the target genome up to the nucleotide level, while NHEJ may result in small insertions/deletions at the junction between the repair nucleic acid molecule and genomic DNA.

As used herein, “a modification of the genome”, means that the genome has been changed by at least one nucleotide (in one embodiment that change does not occur in an unmodified/wild type plant). This can occur by replacement of at least one nucleotide and/or a deletion of at least one nucleotide and/or an insertion of at least one nucleotide, as long as it results in a total change of at least one nucleotide compared to the nucleotide sequence of the preselected genomic target site before modification, thereby allowing the identification of the modification, e.g. by techniques such as sequencing or PCR analysis and the like, of which the skilled person will be well aware.

Further embodiments disclose methods for identifying and/or selecting a wheat plant comprising an allele of a gene contributing positively or negatively to the number of spikelets per spike, respectively comprising the step of identifying the presence or absence, respectively, in the genome of the wheat plant of a nucleic acid having the nucleotides from position 4399 to position 4513 of SEQ ID NO: 5, or of a nucleic acid having the nucleotides from position 7816 to position 7930 in SEQ ID NO: 19, or of a nucleotide sequence having at least 90% sequence identity thereto.

The wheat plants of the present invention may be grown or harvested for grain, primarily for use as food for human consumption or as animal feed, or for fermentation or industrial feedstock production such as ethanol production, among other uses. Alternatively, the wheat plants may be used directly as feed. The plant of the present invention is preferably useful for food production and in particular for commercial food production. Such food production might include the making of flour, dough, semolina or other products from the grain that might be an ingredient in commercial food production. The invention also provides flour, meal or other products produced from the grain. These may be unprocessed or processed, for example by fractionation or bleaching.

The present invention also provides products produced from the plants or grain/seed of the present invention, such as a food product, which may be a food ingredient. Examples of food products include flour, starch, leavened or unleavened breads, pasta, noodles, animal fodder, breakfast cereals, snack foods, cakes, malt, pastries and foods containing flour-based sauces. The food product may be a bagel, a biscuit, a bread, a bun, a croissant, a dumpling, an English muffin, a muffin, a pita bread, a quickbread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food. The food product may be prepared by mixing the grain, or flour, wholemeal or bran from said grain, with another ingredient. Another product is animal feed such as harvested grain, hay, straw or silage. The plants of the invention may be used directly as animal feed, for example when growing in the field.

In one embodiment, the invention provides a method of producing wheat flour, wholemeal, starch, starch granules or bran, the method comprising obtaining the grain of the plant of the invention and processing the grain to produce the flour, wholemeal, starch, starch granules or bran, as well as the wheat flour, wholemeal, starch, starch granules or bran produced by that method or comprising the Apo1 nucleic acid molecule of the invention and/or the APO1 polypeptide of the invention.

Also provided herein is a method of producing a food product, comprising mixing the grain of the plants of the invention or the above wheat flour, wholemeal, starch, starch granules or bran with at least one other food ingredient to produce the food product. Also provided is a method of producing starch, the method comprising obtaining the grain of the plants of the invention and processing the grain to produce the starch, as well as a method of producing ethanol, the method comprising fermenting said starch, thereby producing the ethanol.

Further provided herein is a method of feeding an animal, comprising providing to the animal the wheat plant of the invention, the wheat grain of the invention, the wheat cell of the invention or a feed product comprising the above wheat flour, wholemeal, starch, starch granules or bran.

Also provided is a food product comprising the wheat plant of the invention or a part thereof, the wheat grain of the invention, the wheat cell of the invention, the nucleic acid molecule of the invention, the polypeptide of the invention, or an ingredient which is the above wheat flour, wholemeal, starch, starch granules or bran, such as said food product, wherein the food product is leavened or unleavened bread, pasta, noodle, breakfast cereal, snack food, cake, pastry or a flour-based sauces.

Further provided herein are seeds of the plants of the invention, comprising the Apo1 allele of the invention, as well as a wheat products produced from such seeds, wherein said wheat product comprises the Apo1 allele. Such wheat product can be or can comprise meal, ground seeds, flour, flakes, etc. Particularly, such wheat product comprises a nucleic acid that produces an amplicon diagnostic or specific for the Apo1 allele of the invention.

Also provided herein is a method of altering the number of spikelets per spike of a wheat plant comprising the step of altering the abundance of the APO1 protein of the invention within said wheat plant, particularly such method, wherein the abundance of said protein is increased and the number of spikelets per spike is increased compared to the number of spikelets per spike of said wheat plant where the abundance of said protein is not altered.

The method according to the above paragraph, wherein the abundance of said protein is decreased and the number of spikelets per spike is decreased compared to the number of spikelets per spike of said wheat plant where the abundance of said protein is not altered, such as said method wherein the abundance of said protein is increased by providing said wheat plant with:

a. the recombinant gene of the invention, or
b. a heterologous gene encoding the APO1 protein of the invention, wherein said heterologous gene is higher expressed than the corresponding endogenous gene, e.g., when said heterologous gene comprises the nucleotide sequence of SEQ ID NO: 4 or SEQ ID NO: 5 or a nucleotide sequence having at least 90% sequence identity thereto.

Also provided here is the method of the above 2 paragraphs, wherein the abundance of said protein is decreased by providing said wheat plant with:

a. a heterologous gene encoding the APO1 protein according to the invention, wherein said heterologous gene is lower expressed than the endogenous gene, or
b. a mutant allele of the endogenous gene encoding the protein APO1 according of the invention.

The method of the above paragraph, wherein the promoter of said heterologous gene comprises the nucleotide sequence of SEQ ID NO: 9 or a nucleotide sequence having at least 90% sequence identity thereto, and does not comprise the nucleotide sequence from nucleotide position 4399 to nucleotide position 4513 of SEQ ID NO: 5, nor a nucleotide sequence having at least 90% sequence identity thereto, e.g., wherein said mutant allele is a knock out allele.

The method according to the above paragraphs, wherein the step of providing comprises providing by transformation, crossing, backcrossing, introgressing, genome editing or mutagenesis.

The transformed plant cells and plants obtained by the methods described herein may be further used in breeding procedures well known in the art, such as crossing, selfing, and backcrossing. Breeding programs may involve crossing to generate an F1 (first filial) generation, followed by several generations of selfing (generating F2, F3, etc.). The breeding program may also involve backcrossing (BC) steps, whereby the offspring is backcrossed to one of the parental lines, termed the recurrent parent.

In certain jurisdictions, plants according to the invention, which however have been obtained exclusively by essentially biological processes, wherein a process for the production of plants is considered essentially biological if it consists entirely of natural phenomena such as crossing or selection, may be excluded from patentability. Plants according to the invention thus also encompass those plants not exclusively obtained by essentially biological processes.

The sequence listing contained in the file named “BCS18-2001-WO1_ST25.txt”, which is 87 kilobytes, contains 31 sequences SEQ ID NO: 1 through SEQ ID NO: 31 is filed herewith by electronic submission and is incorporated by reference herein.

In the description and examples, reference is made to the following sequences:

SEQ ID No. 1: nucleotide sequence of the coding DNA of Apo1-7A from Chinese Spring, Westonia or Baxter.
SEQ ID No. 2: nucleotide sequence of the genomic DNA of Apo1-7A from Chinese Spring, Westonia or Baxter.
SEQ ID No. 3: amino acid sequence of the protein APO1-7A from Chinese Spring, Westonia or Baxter.
SEQ ID No. 4: nucleotide sequence of the 5′ upstream sequence of Apo1-7A from Westonia.
SEQ ID No. 5: nucleotide sequence of the 5′ upstream sequence of Apo1-7A from Baxter.
SEQ ID No. 6: nucleotide sequence of the coding DNA of Apo1-7A from Chara or Yitpi.
SEQ ID No. 7: nucleotide sequence of the genomic DNA of Apo1-7A from Chara or Yitpi.
SEQ ID No. 8: amino acid sequence of the protein APO1-7A from Chara or Yitpi.
SEQ ID No. 9: nucleotide sequence of the 5′ upstream sequence of Apo1-7A from Chara or Yitpi.
SEQ ID No. 10: nucleotide sequence of the molecular marker wsnp_Ku_c19943_29512612.
SEQ ID No. 11: nucleotide sequence of the molecular marker Excalibur_c95707_285.
SEQ ID No. 12: nucleotide sequence of the molecular marker mTRI00073530.
SEQ ID No. 13: nucleotide sequence of the molecular marker mTRI00055675.
SEQ ID No. 14: nucleotide sequence of the molecular marker mTRI00055678.
SEQ ID No. 15: nucleotide sequence of the 7B homeologous APO1 gene coding sequence (Chinese Spring).
SEQ ID No. 16: nucleotide sequence of the 7D homeologous APO1 gene coding sequence (Chinese Spring).
SEQ ID No. 17: amino acid sequence of protein APO1-7B (Chinese Spring).
SEQ ID No. 18: amino acid sequence of protein APO1-7D (Chinese Spring).
SEQ ID No. 19: nucleotide sequence of the 5′ upstream sequence of Apo1-7A from Chinese Spring.
SEQ ID No. 20: 1242 nucleotide sequence of the coding DNA of Apo1-7B from Chinese Spring.
SEQ ID No. 21: nucleotide sequence of the genomic DNA of Apo1-7B from Chinese Spring.
SEQ ID No. 22: nucleotide sequence of the 5′ upstream sequence of Apo1-7B from Chinese Spring.
SEQ ID No. 23: nucleotide sequence of marker CAP7_c2350_105.
SEQ ID No. 24: nucleotide sequence of marker wsnp_Ku_rep_c104159_90704469.
SEQ ID No. 25: nucleotide sequence of marker BS00021657_51.
SEQ ID No. 26: nucleotide sequence of marker BS00066288_51.
SEQ ID No. 27: nucleotide sequence of marker BS00039502_51.
SEQ ID No. 28: nucleotide sequence of the coding DNA of Apo1-7A from Chinese Spring (shorter version).
SEQ ID No. 29: amino acid sequence of the protein APO1-7A from Chinese Spring (shorter version).
SEQ ID No. 30: nucleotide sequence of the coding DNA of Apo1-7B from Chinese Spring (shorter version).
SEQ ID No. 31: amino acid sequence of the protein APO1-7B from Chinese Spring (shorter version).

### EXAMPLES

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany. Standard procedures for AFLP analysis are described in Vos et al. (1995, NAR 23:4407-4414) and in published EP patent application EP 534858.

The Examples show results obtained using 2 different wheat populations, one based on analysis of a group of spring wheat plants (section A below) and one based on the analysis of a group of winter wheat plants (section B below), showing that the identified SPS phenotype (SPS− or SPS+) linked to the type of APO1 allele present is applicable across all wheat populations/genotypes.

### A. APO1 Analysis in Spring Wheat Lines

### Example 1: Mapping of a QTL on Chromosome 7A Controlling the Number of Spikelets Per Spike

A 4-way MAGIC spring wheat population (Huang et al. 2012 Plant Biotechnology Journal 10:826-839) was phenotyped by counting the number of spikelets per spike on the different plant lines.

Using a genetic map of several SNP, QTL analysis was carried out to test the effect of variation in spikelet number per spike across all markers. Significant marker-trait associations are distinguished by—log-transformed p-values higher than 3. In this way, an interval of significantly associated markers was delineated, including flanking markers (SEQ ID NO. 10 and SEQ ID NO. 11). An interval of significantly associated markers was delineated using the following criteria: significance threshold at 2.5, significance drop at 1.5 and significance drop between peaks at 2. This delimited the interval to 2.1 cM for 7A by the left and right flanking markers.

Heterogeneous inbred families (HIFs) with contrasting presence of the 7A SPS QTL (Fam1_A_1, Fam1_B_1, Fam2_B_1, Fam2_C_1, Fam2_H_1, Fam3_E_1, Fam3_I_1, Fam_4_A, Fam4_G, Fam5_C_1 and Fam5_F_1) have been generated and were subsequently used for fine mapping and the expression analysis below of the 7A QTL.

The HIFs with contrasting presence of the high and low contributing alleles for the 7A SPS QTL were phenotyped as described above. Additional SNP assays were developed to increase the marker density in the QTL interval. The SPS locus could be further delimited to a region of about 2.1 cM on 7A (from 58.7 to 60.8 cM along chromosome 7A) delimited by flanking markers (SEQ ID NO: 12 and SEQ ID NO: 13 or SEQ ID NO: 14).

Sequence of fine-mapped markers was used for BLASTs to contigs and scaffolds of genome sequence of Chinese Spring. Stringent BLAST and parsing criteria were applied to position the SNPs in the partial genome sequence, such as >98% sequence identity, alignment length of >158 bp, hit in 7A sequence, and additional criteria for non-aligning overhang. Scaffolds were ordered to the fine map (and additional genetic maps). 16 annotated genes within the interval defined by the fine mapping, were subjected to expression analysis as described in Example 2.

### Example 2: Expression Analyses and Identification of APO1

Expression analysis was performed using whole transcriptome shotgun sequencing of RNA samples prepared from the contrasting HIF families, essentially as described by Wang et al. (2009) Nature Review Genetics 10, 57-63. Expression was quantified by counting the normalized number of reads that mapped to the QTL interval defined in Example 1.

The expression level of the 16 genes annotated in the interval defined by the fine mapping has been quantified in the different parents of the mapping population as well as in 11 HIFs. Of these, only one candidate, the ortholog of the rice APO1, is significantly higher expressed (with an average of 1.8 fold increase) in the lines displaying the phenotype of high number of spikelets per spike (abbreviated herein at times as SPS+ (phenotype)) compared to lines having a low number of spikelets per spikes (abbreviated herein at times as SPS− (phenotype”). This gene was consequently identified as the gene underlying the number of spikelets per spike QTL on the chromosome 7A.

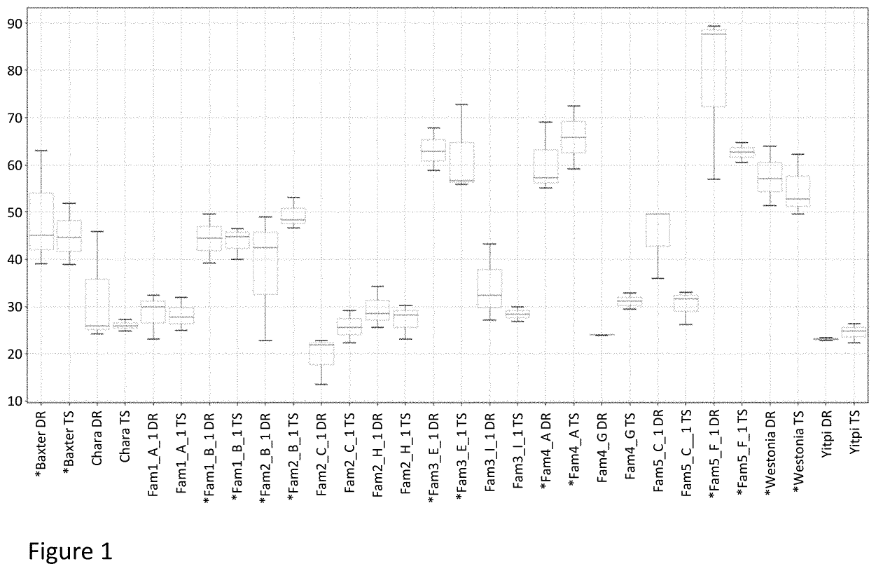

FIG. 1 shows the detailed results of the expression level by RNAseq transcription analysis of APO1 gene in the analyzed spring wheat genotypes. The contrasting lines have a minimum of 1.5 fold and up to a 2.75 fold difference in APO1 transcript abundance. The parents Chara and Yitpi have a low number of spikelets per spike and a low expression level of APO1, while the parents Westonia and Baxter have a high number of spikelets per spike and have a higher expression level of APO1 (1.6 to 2.6 fold higher). Similarly the HIFs lines having a low number of spikelets per spike have a low expression level of APO1 while the HIFs lines having a high number of spikelets per spike have a higher expression level of APO1.

The sequence of the APO1 gene was obtained from the reference wheat line Chinese Spring as well as from the four MAGIC parent varieties. APO1 is very well conserved with more than 99% sequence identity between the sequence of the allele from the low spikelets per spike varieties and between the sequence of the allele from the high spikelets per spike varieties. Table 1 shows the 3 single nucleotide polymorphisms found between the APO1 coding sequences analyzed. The corresponding amino acid sequences also share 99% of sequence identity. The SNP at position 140 on SEQ ID NOs: 2 or 7 results in the the Yitpi and Chara protein sequence (SEQ ID NO: 8) having a cysteine at position 47, while the Baxter, Westonia and Chinese Spring protein sequences (SEQ ID NO: 3) have a phenylalanine at position 47. The SNP at position 842 on SEQ ID NOs: 2 or 7 does not result in any difference in the amino acid sequences as it is in an intron. The SNP at position 1284 on SEQ ID Nos: 2 or 7 results in the Yitpi and Chara protein sequence (SEQ ID NO: 8) having an asparagine at position 384, while the Baxter, Westonia and Chinese Spring protein sequences (SEQ ID NO: 3) have an aspartic acid at position 384. These differences in the protein sequences of the high and the low spikelets per spike genotypes are not expected or predicted to significantly alter the function of the APO1 protein.

TABLE 1
Single nucleotide polymorphisms (SNPs) identified between the
APO1 gene sequences of the varieties having low number of spikelets
per spike (Yitpi and Chara) and the varieties having high number
of spikelets per spike (Baxter and Westonia).

| Position | | | |
|---|---|---|---|
| SEQ ID NO: | Yitpi/ | Baxter/ | |
| 2 or 7 | Chara | Westonia | Type |
| 140 | G | T | SNP |
| 842* | T | C | SNP |
| 1284 | A | G | SNP |

*refers to a SNP in an intron sequence.

The about 5 kb nucleotide sequence upstream of the APO1 gene was also obtained and compared from the four parent varieties. Table 2 lists single nucleotide polymorphisms and the insertion/deletions found between the sequences from the low spikelets per spike genotypes and the sequences from the high spikelets per spike genotypes. Strikingly, the sequences from the genotypes of the varieties having a low number of spikelets per spike are missing about 115 bp compared to the sequences from the genotypes of the varieties having a high number of spikelets per spike at about 500 bp upstream of the translation start site (corresponding to the translation start site in the reference sequence of SEQ ID NO: 1). This deletion is expected to explain the lower expression level measured in those lines.

TABLE 2
Single nucleotide polymorphisms (SNPs) and insertion/deletions
(Indel) identified between the about 5 kb upstream sequences
of APO1 of the varieties having low number of spikelets per
spike (Yitpi and Chara) and the varieties having high number
of spikelets per spike (Baxter and Westonia).

| Position | | | Position | Position | |
|---|---|---|---|---|---|
| SEQ ID | Yitpi/ | Baxter/ | SEQ ID | SEQ ID | |
| NO: 9 | Chara | Westonia | NO: 4 | NO: 5 | Type |
| 32 | G | A | 32 | 32 | SNP |
| 33 | G | A | 33 | 33 | SNP |
| 520 | G | T | 520 | 520 | SNP |
| — | — | T | 551 | 551 | indel |
| 651 | A | G | 652 | 652 | SNP |
| 1063 | A | — | — | — | indel |
| 1482 | T | C | 1482 | 1482 | SNP |
| 1639 | C | T | 1639 | 1639 | SNP |
| 2093 | T | — | — | — | indel |
| 2094 | C | — | — | — | indel |
| 2095 | T | — | — | — | indel |
| 2096 | C | — | — | — | indel |
| 2097 | T | -/T | 2093 | — | indel |
| 2098 | C | -/C | 2094 | — | indel |
| 2660 | A | G | 2656 | 2654 | SNP |
| 2730 | A | C | 2726 | 2724 | SNP |
| 2747 | A | G | 2743 | 2741 | SNP |
| 2759 | T | G | 2755 | 2753 | SNP |
| 2785 | C | T | 2781 | 2779 | SNP |
| 2792 | T | C | 2788 | 2786 | SNP |
| 3000 | T | C | 2996 | 2994 | SNP |
| 3241 | G | A | 3237 | 3235 | SNP |
| 3456 | C | T | 3452 | 3450 | SNP |
| 3493 | C | T | 3489 | 3487 | SNP |
| 3603 | G | A | 3599 | 3597 | SNP |
| — | — | G | 4108 | 4106 | indel |
| — | — | C | 4109 | 4107 | indel |
| — | — | CAATTTACTCTAGTT | 4401-4515 | 4399-4513 | indel |
| | | GCATCCCAACATCG | | | |
| | | TGCCCCTACCTCGC | | | |
| | | CTCCGGCTAGGTCA | | | |
| | | TTCCAAGCCCTAGTC | | | |
| | | GCCGACGTCGCAAC | | | |
| | | CCTGTCTCATGCTC | | | |
| | | GGCGGCTATCTAATT | | | |
| 4403 | A | C | 4516 | 4514 | SNP |
| 4427 | G | A | 4540 | 4538 | SNP |
| 4643 | A | G | 4756 | 4754 | SNP |
| 4753 | G | T | 4866 | 4864 | SNP |

The SNPs and indels identified between the high and the low spikelets per spike genotypes may also be used as markers to determine which allele of the APO1 gene is comprised with any particular wheat genotype.

Growing 2 Spring wheat NIL Lines (NILs) contrasting at the APO1-7A locus in different environments showed that the APO1-7A allele causing a reduced number of spikelets per spike (SPS−) was linked to a significant yield increase in field trials (between 3 and 6 replicates for each line under testing) when grown in Australia, compared to the contrasting NILs carrying the APO1-7A allele causing increased number of spikelets per spike (SPS+) in the same genetic backgrounds (grown in the same trials). This association was reversed when the same NILs were grown in field trials in France (between 3 and 6 replicates for each line under testing), where the lines having the APO1 allele causing increased number of spikelets per spike (SPS+) showed a significant yield increase, compared to the sibling lines having the APO1 SPS-allele in the same genetic backgrounds (grown in the same trials). Whilst the yield effects were reversed, the effects of each of the 2 APO1-7A alleles for SPS phenotype were consistent across environments.

### Example 3: Validation of APO1 as the Spikelets Per Spike Determining Gene in Wheat Plants Having Initial Low Spikelets Per Spike Number (GM Approach)

Using standard recombinant DNA techniques, the following DNA regions were operably linked:

a. a CaMV35S promoter region (P35S)
b. A DNA region encoding TaAPO1
c. A DNA region representing the 3′ untranslated sequence OCS terminator
The recombinant gene was introduced into a T-DNA vector which contains a selectable marker cassette to result in the T-DNA vector P35S::APO1.

Using standard recombinant DNA techniques, the following DNA regions were operably linked:

a. a Ubiquitin promoter region (PUbi)
b. A DNA region encoding TaAPO1
c. A DNA region representing the 3′ untranslated sequence OCS terminator
The recombinant gene was introduced into a T-DNA vector which contains a selectable marker cassette to result in the T-DNA PUbi::APO1.

Using standard recombinant DNA techniques, the following DNA regions were operably linked:

a. the about 5 kb promoter region of APO1 from the wheat variety Westonia (SEQ ID NO: 4)
b. A DNA region encoding TaAPO1
c. A DNA region representing the 3′ untranslated sequence OCS terminator
The recombinant gene was introduced into a T-DNA vector which contains a selectable marker cassette to result in the T-DNA Papo1::APO1.

The three T-DNA vectors were introduced into Agrobacterium comprising helper Ti-plasmids using standard techniques and are used in wheat transformation essentially as described in Ishida et al. 2015 Agrobacterium protocols: Volume 1, Methods in Molecular Biology, vol. 1223: 189-198. Either directly Chara or Yitpi is transformed, or any other variety is transformed and then used as donor to introduce the recombinant gene in Chara or Yitpi variety by crossing and selecting. The wheat variety Fielder is used as control for the transformation efficiency. The Fielder transformants are also phenotyped to assess the effect of the APO1 gene over-expression on spikelets per spike. The Fielder transformants can be used for introgressing the recombinant gene into Chara or Yipti.

Independent events are obtained from each transformation and are phenotyped according to the method described in Example 1.

### Example 4: Identification of APO1 Homeologs in Wheat

Using the nucleotide sequence of the APO1 encoding gene located on chromosome 7A, homeologous nucleotide sequences could be detected which are located on chromosome 7B and 7D respectively in the Chinese Spring wheat reference genomes. The nucleotide sequences for the coding regions of these genes are included in sequence listing entries SEQ ID NO: 15 (7B Apo1) and 16 (7D Apo1), respectively. The amino acid sequences are included in Sequence listing entries SEQ ID NO: 17 (7B Apo1) and SEQ ID NO: 18 (7D Apo1). According to a shorter gene model for 7B Apo1, the nucleotide sequence corresponds to SEQ ID NO: 15 from nucleotide 130 to nucleotide 1452 and the amino acid sequence corresponds to SEQ ID NO: 17 from amino acid 45 to amino acid 483.

The respective sequence identities of the nucleotide sequences of the coding sequences are represented in Table 3 while those of the amino acid sequences of the encoded proteins are found in Table 4.

TABLE 3
% sequence identity between Apo1 homoelogous genes.

| | | | Apo1 7B short | |
|---|---|---|---|---|
| | Apo1 7A | Apo1 7B | (SEQ ID | Apo1 7D |
| | (SEQ ID | (SEQ ID | NO: 15 | (SEQ ID |
| | NO: 1) | NO: 15) | from nt 130) | NO: 16) |
| Apo1 7A | 100 | | | |
| (SEQ ID NO: 1) | | | | |
| Apo1 7B | 88 | 100 | | |
| (SEQ ID NO: 15) | | | | |
| Apo1 7B short | 97 | 91 | 100 | |
| (SEQ ID NO: 15 | | | | |
| from nt 130) | | | | |
| Apo1 7D | 96 | 87 | 96 | 100 |
| (SEQ ID NO: 16) | | | | |

TABLE 4
% sequence identity between Apo1 proteins
encoded by the homoelogous genes.

| | | | Apo1 7B short | |
|---|---|---|---|---|
| | Apo1 7A | Apo1 7B | (SEQ ID | Apo1 7D |
| | (SEQ ID | (SEQ ID | NO: 17 | (SEQ ID |
| | NO: 3) | NO: 17) | from aa 45) | NO: 18) |
| Apo1 7A | 100 | | | |
| (SEQ ID NO: 3) | | | | |
| Apo1 7B | 89 | 100 | | |
| (SEQ ID NO: 17) | | | | |
| Apo1 7B short | 97 | 90 | 100 | |
| (SEQ ID NO: 17 | | | | |
| from aa 45) | | | | |
| Apo1 7D | 97 | 88 | 97 | 100 |
| (SEQ ID NO: 18) | | | | |

### B. APO1 Analysis in Winter Wheat Lines

### Example 1: Rough Mapping of a QTL on Chromosome 7A Controlling the Number of Spikelets Per Spike

### Phenotyping

fully replicated trial of 784 F₇ MAGIC lines from the winter wheat MAGIC population of Mackay et al. (2014, G3-Genes Genomes Genetics, 4(9): 1603-1610) and their eight founders was grown during the 2013/2014 field season. Ten representative wheat ears were collected from 1000 of the 1600 plots in the field, and dried at room temperature. Collection was done in a partially replicated design with 200 RILs and the MAGIC parents collected in duplicate. The wheat ears were screened for the morphology trait of total spikelet number per spike (abbreviated as “SPS”).

In 2014/2015 a nursery of 1091 F₈ MAGIC lines and the founders was screened for the same spike traits using a sample of six representative wheat ears per plot.

Asreml-R 3.0 (Gilmour et al. 1997, Journal of Agricultural Biological and Environmental Statistics Vol 2(3), 269-293) was used to minimize or remove spatial effects in phenotype data due to field variation. While the mpwgaim QTL analysis package allows for a one stage fitting of QTLs, the other QTL analysis packages used in this research required prior calculation of trait BLUPS (Best Linear Unbiased Predictions).

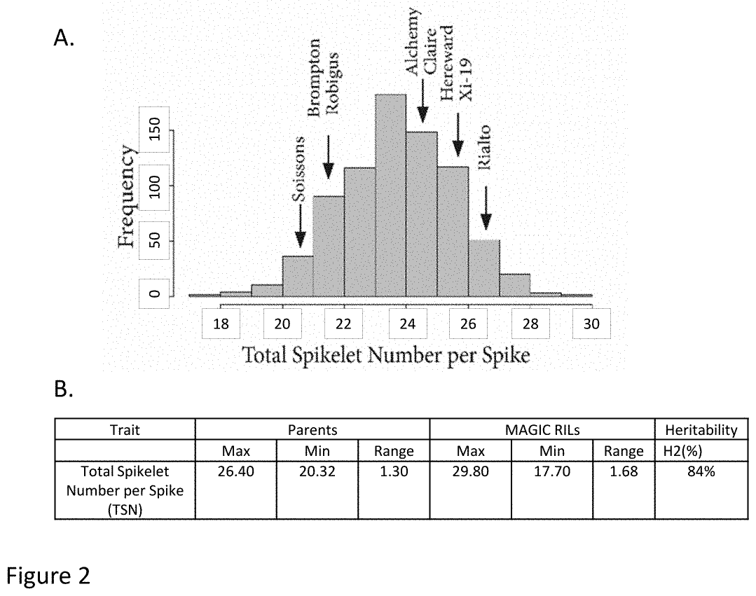

Total spikelet number varied between 18 and 30 spikelets per spike in the RILs. The MAGIC parents can broadly be divided into a high and low phenotype group, with Soissons, Robigus and Brompton having a reduced number of spikelets compared to the other five MAGIC parents (FIG. 2). The Soissons mean phenotype is even lower than Robigus and Brompton and only 2.6 spikelets greater than the recorded minimum phenotype in the RILs (Recombinant Inbred Lines). The reduced total spikelet number in Soissons is related to the fact that unlike the other varieties, it possesses the photoperiod insensitive Ppd-D1 allele which confers both earlier flowering and also reduced spikelet number (Gonzalez et al, 2005, Euphytica 146(3):253-269). The other 7 MAGIC parents do not carry this allele and thus the basis for reduced spikelet number in Robigus and Brompton was not related to that Ppd-D1 allele.

### Genetic Mapping

QTL analyses were conducted using three different methodologies: (i) using simple regression of line means with marker scores while accounting for the MAGIC crossing funnel structure (Mackay et al. 2014) using the R package Asreml-R (Gilmour, 1997), (ii) Bayesian network analysis using the R package bnlearn (Scutari et al., 2014, Genetics, 198(1):129-137)) and (iii) Whole genome average interval mapping using the R package mpwgaim (Verbyla et al., 2014, G3, 4(9):1569-1584).

Marker genotypes and their respective chromosomal groupings from Gardner et al., 2016 (2016, Plant Biotechnol J, 14(6):1406-1417) were used.

All three methods identified a major QTL on chromosome 7A between 257.05 cM and 257.21 cM on the MAGICmapv14.4, hereafter termed QTsn.jbl-7A (Table 5).

TABLE 5
Summary of significant QTLs identified for total spikelet number (SPS) using Regression [17], Bayesian Network
analysis [23] or Genome wide interval mapping [22]. The peak marker in regression analysis is the marker
with the lowest or joint lowest p-value. Significant markers may extend further away from the Peak marker shown. Mpwgaim
reports p values < 0.0005 as 0. Regression q values of 0 are <2.2E−16. Abbreviations: Chromosome (chr) and centiMorgan (cM).

| | | | | q-value | p-value | | | |
|---|---|---|---|---|---|---|---|---|
| Method | Peak Marker/Left Marker | Chr | cM | Regession | mpwgaim | Right Marker | cM | % var |
| Regression | wsnp_Ku_rep_c104159_90704469 | 7A | 257.21 | 0 | | | | |
| mpwgaim | CAP7_c2350_105 | 7A | 257.05 | | 0 | BS00021657_51 | 257.21 | 35 |
| Bnlearn | wsnp_Ku_rep_c104159_90704469 | 7A | 257.21 | | | | | |
| mpwgaim | BS00066288_51 | 7B | 144.34 | | 1.00E−03 | BS00039502_51 | 144.50 | 1.9 |

### Marker Info

CAP7_c2350_105
(https://triticeaetoolbox.org/wheat/view.php?table=markers&name=CAP7_c2350_105)
(SEQ ID NO: 23)
TAGTAAGCTCTTCAACGAGGATGGATGTTGTGTAATTTGGACAAGTGCGA[C/T]GTATGTCAC
ATCTTTTTTTTAATGATCCTAATCTATGATCGAAGTTCGTT.
wsnp_Ku_rep_c104159_90704469
https://triticeaetoolbox.org/wheat//view.php?table=markers&name=IWA7409
(SEQ ID NO: 24)
TGCCGGCCTGCAAGCCGATCCTTACTCCAAARTGGGTTGTCTCGGTGTTTTTCCTTGTCGGCGTCGTCTTTGTCCCAG
TTGGTGTCGTTTCGCTACTAGC[C/T]gcacaagatgttgttgagatcattgatcggtatgatcatgcatgtgtccca
cctaacatgactgataacaagcttgcgtacatccagaatgagactatac.
Marker BS00021657_51
https://triticeaetoolbox.org/wheat//view.php?table=markers&name=BS00021657_51
(SEQ ID NO: 25)
TCCACAAGAAAAGAGCAAGACACTCCGGCCGTTGTAGAGCTGATGGTGCG[C/T]GGTGATTT
CACCATAGACATGGTAGACGGCGCCCGTCCTCGTGGCATCAT.
Marker BS00066288_51
https://triticeaetoolbox.org/wheat///view.php?table=markers&name=BS00066288_51
(SEQ ID NO: 26)
GGCACGTACTCCCTTTCAGGACCCGACGAACAACGGCAATTCAGGTAAAT[A/G]CATACATC
ACGTACTCTTACATACTTCAATCTTGTAAATCCATAATATAT.
Marker BS00039502_51
https://triticeaetoolbox.org/wheat//view.php?table=markers&name=BS00039502_51
(SEQ ID NO: 27)
ATCCCAGGGGGCGAGATTCAGAGCTTCTCGGCCATCCTGCGCAGCAGCGC[A/G]GCCCCTAG
TGGCTCCTCGGTCGGGTTCTTGGTGAGCCATGCCTGCGCGGC.

QTsn.jbl-7A explains a remarkable 35% of genetic variation in SPS within the MAGIC population at a −log10(p) of 62.64 using mpwgaim. Spikelet phenotypes collected from the MAGIC nursery in 2015 confirmed the presence of the QTL with a −log10(p) of 37.82 for SPS using mpwgaim (Table 6).

TABLE 6
Mpwgaim QTL results for 2015 MAGIC nursery total spikelet number. Abbreviations:
LOGP is −log10(p), % Var is percentage of genetic variation explained.

| | | DIST | | DIST | | | |
|---|---|---|---|---|---|---|---|
| CHROMOSOME | LEFT MARKER | (CM) | RIGHT MARKER | (CM) | PROB | % VAR | LOGP |
| 7A | CAP7_c2350_105 | 257.05 | BS00021657_51 | 257.21 | 0 | 19.2 | 37.82 |

The Brompton and Robigus haplotypes cause a relative reduction of the progenies' SPS by more than 1.5 spikelets in both 2014 and 2015 (Table 7).

TABLE 7
Total number of spikelets per spike QTL summary for QTsn.jbl-7A. 2014 NIAB MAGIC yield trial
phenotype data used. Estimated parental haplotype effects on RIL BLUPs from mpwgaim analysis. Abbreviations:
LOGP is −log₁₀(p). 2 and 0 are allele codes for the respective markers shown.

| | Founder | Founder | Founder | % var | | | |
|---|---|---|---|---|---|---|---|
| Founder | effects | Probability | LOGP | explained | LOGP | CAP7_c2350_105 | wsnp_Ku_rep_c104159_90704469 |
| Alchemy | 0.634 | 0.083 | 1.08 | 35 | 62.64 | 2 (GG) | 0 (AA) |
| Brompton | −1.607 | 0 | 5.31 | | | 0 (AA) | 2 (GG) |
| Claire | 0.566 | 0.102 | 0.99 | | | 2 (GG) | 0 (AA) |
| Hereward | 0.559 | 0.065 | 1.19 | | | 2 (GG) | 0 (AA) |
| Rialto | 0.242 | 0.273 | 0.56 | | | 2 (GG) | 0 (AA) |
| Robigus | −1.766 | 0 | 6.14 | | | 0 (AA) | 2 (GG) |
| Soissons | 0.01 | 0.489 | 0.31 | | | 2 (GG | 0 (AA) |
| Xi-19 | 1.007 | 0.005 | 2.31 | | | 2 (GG) | 0 (AA) |

The 0.16 cM genetic mapping interval corresponds to a predicted physical length of ca. 2.3Mb and the flanking markers CAP7_c2350_105 (SEQ ID NO: 23) and wsnp_Ku_rep_c104159_90704469 (SEQ ID NO: 24). Increased total spikelet number most closely co-segregates with the wsnp_Ku_rep_c104159_90704469 marker.

In addition to the QTsn.jbl-7A, QTL analysis with mpwgaim confirmed another QTL on 7B (QTsn.jbl-7B) for total spikelet number in 2014 (LOGP 3.07) between the flanking markers BS00066288_51 (144.34 cM; SEQ ID NO: 26) and B500039502_51 (144.50 cM; SEQ ID NO: 27), which define a 5 Mb interval directly homoeologous to the 7A QTL (see Table 8).

TABLE 8
Total number of spikelets per spike QTL summary for QTsn.jbl-7B. 2014 MAGIC yield trial phenotype
data used. Estimated parental haplotype effects on RIL BLUPs from mpwgaim analysis. Abbreviations:
LOGP is −log10(p). 2 and 0 are allele codes for the respective markers shown.

| | Founder | Founder | Founder | % var | | | |
|---|---|---|---|---|---|---|---|
| Founder | effects | Probability | LOGP | explained | LOGP | BS00039502_51 | BS00066288_51 |
| Alchemy | −0.37 | 0.012 | 1.92 | 1.9 | 3.07 | 0 (TT) | 0 (TT) |
| Brompton | −0.198 | 0.12 | 0.92 | | | 0 (TT) | 0 (TT) |
| Claire | 0.283 | 0.048 | 1.32 | | | 2 (CC) | 0 (TT) |
| Hereward | −0.043 | 0.395 | 0.4 | | | 0 (TT) | 0 (TT) |
| Rialto | −0.052 | 0.365 | 0.44 | | | 0 (TT) | 2 (CC) |
| Robigus | −0.12 | 0.214 | 0.67 | | | 0 (TT) | 2 (CC) |
| Soissons | 0.187 | 0.11 | 0.96 | | | 0 (TT) | 0 (TT) |
| Xi19 | 0.289 | 0.044 | 1.36 | | | 2 (CC) | 0 (TT) |

### Example 2: Identification of the Candidate Gene APO1

### 25 Candidate Genes in QTsn.jbl-7A

Within this 2.3 Mb interval 25 genes were annotated. Orthologue identification revealed seven genes with well annotated orthologues and functions: g109255 (AtFTT/AtDTX35), g109235 (AtRAN1), g109240 (AtCHLI), g109250 (AtAAH), g109253 (AtSYP132), g109256 (AtALIS4) and g109251 (AtUFO). AtUFO is the orthologue of rice APO1 (ABERRANT PANICLE ORGANIZATION 1). A further ten genes had redundant annotations as At5g07610 related F-box proteins. Each contained an F-box domain and shows considerable DNA sequence conservation of up to 72.5% between themselves.

### Synteny Analysis of QTsn.jbl-7A Reveals APO1 as a Candidate Gene

In addition to the QTsn.jbl-7A (Table 8), QTL analysis with mpwgaim confirmed another QTL on 7B (QTsn.jbl-7B) for total spikelet number in 2014 (LOGP 3.07) between the flanking markers BS00066288_51 (144.34 cM) and BS00039502_51 (144.50 cM), which define a 5 Mb interval directly homoeologous to the 7A QTL.

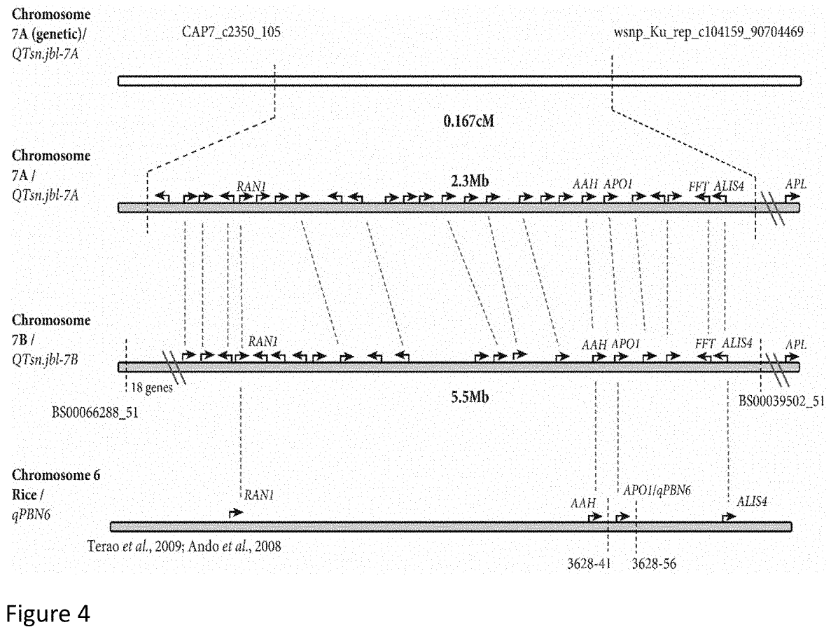

Within the 5 Mb interval of the QTL on 7B (QTsn.jbl-7B) 39 genes were identified, of which 15 were homoeologous to 7A (FIG. 4). Of the 15 homoeologous genes, none has an identifiable deleterious coding sequence mutation as predicted with PROVEAN which could explain both the QTL.

QTsn.jbl-7A and QTsn.jbl-7B are syntenic to rice chromosome 6, which contains four positionally conserved orthologues chr7A.g109235 (AtRAN1), g109250 (AtAAH), g109251 (APO1/AtUFO) and g109256 (AtALIS4).

### Sequence Polymorphisms of TaAPO1-7A Segregate Together with QTsn.jbl-7A

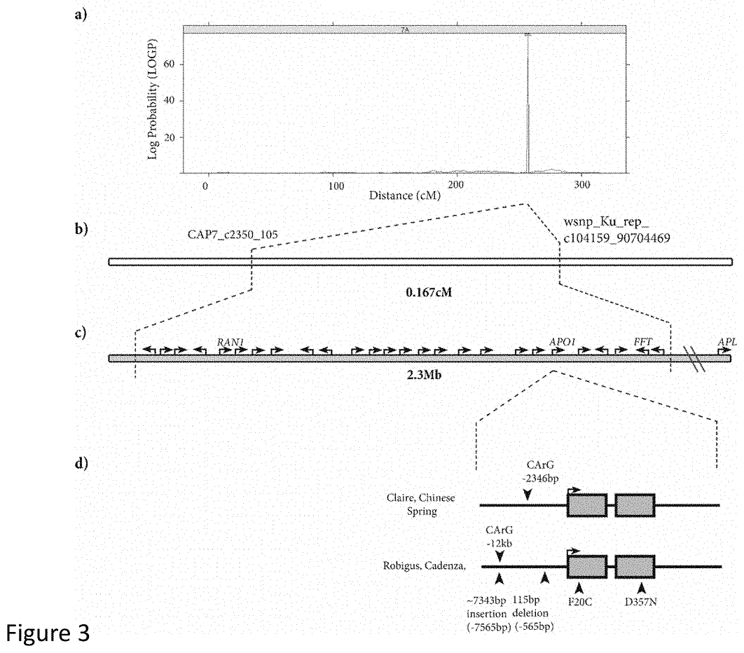

TaAPO1-7A has two large InDels upstream of the predicted transcription start site in Robigus compared to Claire and Chinese spring: a 115 bp deletion 565 bp upstream and an about 5-7.5 Kb insertion (7343 bp, but 4970 bp excluding N/X-runs, size varies based on quality of reference sequence used) about 7 Kb (7565bp upstream of the transcription start site, 7513 bp upstream of the start codon by reference to the sequence of SEQ ID NO: 1 (CS ref sequence) upstream of the transcription start site (TSS). The 115 bp deletion is also present in the wheat varieties Cadenza and Paragon, segregating together with BA00589872 in the 35 k Breeders array. The long insertion in Robigus, Cadenza and Paragon about 7 Kb upstream of the TSS is more difficult to characterize due to some missing base calls in the Robigus, Cadenza and Paragon TGAC assemblies, but a similar large (>5 Kb) insertion is also present in the varieties Yitpi and Chara. The Claire promoter carries one CArG box (CC(A/T)₆GG) 2346 bp upstream, which is absent in Robigus. The about 5-7.5 kb insertion also carries a CArG box (FIG. 3). In addition Robigus has the same SNPs and indels as varieties Yipti/Chara in Table 2, while Claire has the same SNPs and indels as Westonia in Table 2.

When comparing the about 5 kb nucleotide sequence upstream of the APO1 gene in other wheat lines, the variations shown in Table 2 were also found in Robigus, Claire, Cadenza, Paragon and Fielder. Claire has the same SNP and indel alleles as Westonia, Fielder has the same SNP and indel alleles as Baxter. Robigus, Cadenza and Paragon have the same SNP and indel alleles as Yitpi/Chara. This confirms that the sequences from the genotypes of the winter wheat varieties having a low number of spikelets per spike are missing about 115 bp compared to the sequences from the genotypes of the winter wheat varieties having a high number of spikelets per spike at about 500 bp upstream of the translation start site (by reference to the SEQ ID NO: 1 translation start site). This promoter deletion is expected to explain the lower expression level measured in those lines. The about 5-7.5 upstream insertion identified in Robigus, Cadenza and Paragon (7565 bp upstream of the TSS) is also common to Yitpi and Chara.

The amino acid changes (F20C, D357N) associated with SNPs in TaAPO1-7A in Robigus are predicted by PROVEAN to be non-deleterious.

### Example 3: TaAPO1-7A Expression Correlated with Total Spikelet Number

Three replicates of whole spike samples were collected from tiller dissections of the 2017 NIAB MAGIC Nursery at growth stage gS32 (Zadoks et al., 1974, Weed Research, 14(6): 415-421) for the MAGIC parents Alchemy, Brompton, Claire, Hereward, Rialto Robigus and Xi-19. At the collection date Soissons had advanced to gS34. Following dissection spikes were immediately frozen in liquid nitrogen. Primers were designed using the Primer3 (Koressaar et al., 2007, Bioinformatics, 23(10): 1289-1291) plugin in Geneious. Samples were twice homogenised whilst frozen with 5 mm stainless steel beads on the TissueLyser II (QIAGEN, UK) at 20 Hz for two minutes. RNA was extracted using the RNeasy Micro extraction kit (QIAGEN, UK) and DNA digestion was carried out on column using the RNase-free DNase set (QIAGEN, UK). RNA was eluted using RNase/DNase free water and concentration determined using the NanoDrop 1000 spectrophotometer (Thermo Scientific, UK). A second DNA digest was performed using ezDNase (Invitrogen, UK) followed by cDNA synthesis from 500 ng RNA using the SuperScript IV Vilo Master Mix cDNA synthesis kit (Invitrogen, UK). RT-qPCR was performed using the Rotor-Gene SYBR Green PCR Kit on a Rotor-Gene Q Real-Time PCR machine fitted with a Rotor-Disc 100 (QIAGEN, UK). All reactions were carried out as technical duplicates at 10 μl final reaction volume, for APO/betaine solution (Sigma-Aldrich) was added at a final concentration of 1M to overcome the amplicons high GC content. Amplification efficiencies of primer pairs were determined by performing an eight point two-fold serial dilution series of cDNA samples. To confirm specificity of RT-qPCR reactions the melt curves for each reaction were checked for the presence of only a single peak. Specificity of the assays was confirmed against genomic nullitetrasomic DNA obtained from Seedstor.ac.uk (WPGS1289-PG-1, WPGS1296-PG-1, WPGS1301-PG-1. Expression levels of APO1 were calculated relative to the expression of the housekeeping genes TaRP15 (Shaw et al., 2012, Plant J, 71(1): 71-84) and Ta2291 (Paolacci et al., 2009, BMC Molecular Biology, 10(1): 11) using the amplification efficiencies calculated for each assay.

Expression of TaAPO1 in Xi-19 was found to be the highest of all MAGIC founders. The Xi-19 haplotype is also statistically significantly associated with a positive founder effect on QTsn.jbl-7A in 2014 (Table 8).

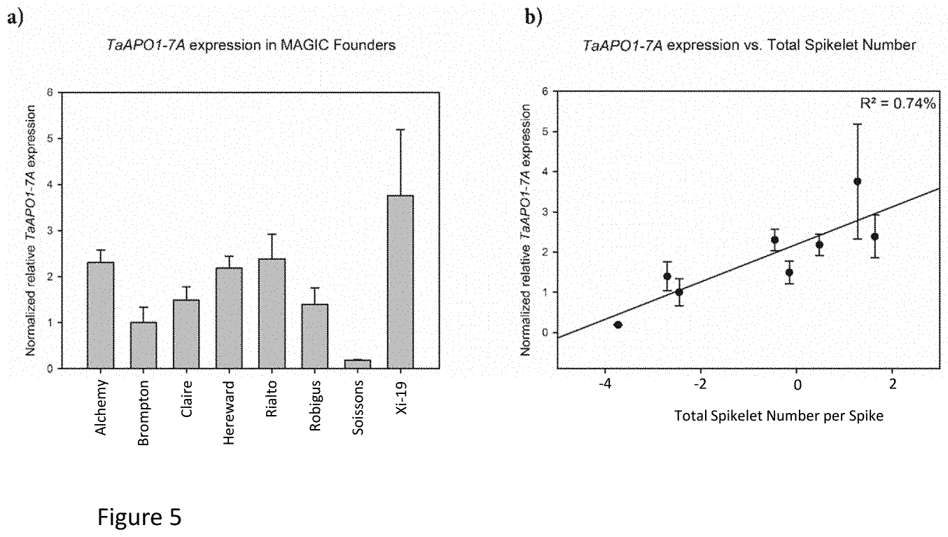

FIG. 5 shows the results of the expression level of APO1 in the studied genotypes. The contrasting lines Brompton and Xi-19 have up to a 3.8 fold difference in APO1 transcript abundance.

1. A protein involved in determining the number of spikelets per spike in wheat which is orthologous to “Aberrant panicle organization 1” (APO1) from rice.

2. The protein according to claim 1 comprising an amino acid sequence selected from:
a. an amino acid sequence of SEQ ID NO: 3, 8, or 29 or a functional variant thereof, or
b. an amino acid sequence having at least 85% sequence identity with the amino acid sequence of SEQ ID NO: 3, 8 or 29, or a functional variant thereof.

3. An isolated nucleic acid encoding the protein according to claim 1 or 2.

4. The nucleic acid according to claim 3 comprising a nucleotide sequence selected from:
a. the nucleotide sequence of any one of SEQ ID NO: 1 or SEQ ID NO: 2,
b. a nucleotide sequence having at least 80% identity to the nucleic acid sequence of any one of SEQ ID NO: 1 or SEQ ID NO: 2;
c. the nucleotide sequence of SEQ ID NO: 6, 7 or 28;
d. a nucleic acid having a complementary sequence to any one of the nucleic acids of a) or b).

5. The nucleic acid according to claim 3 or 4 which localizes within an interval on wheat chromosome 7A comprising the nucleotide sequence comprised between the nucleotide at position 674,081,462 and the nucleotide at position 674,082,918 of the Chinese Spring reference genomic sequence.

6. A recombinant gene comprising a plant expressible promoter, such as a heterologous plant expressible promoter, operably linked to a nucleic acid sequence encoding the protein of claim 1 or 2 and optionally, a transcription termination and polyadenylation sequence, preferably a transcription termination and polyadenylation region functional in plants.

7. The recombinant gene of claim 6, wherein said nucleic acid is selected from:
a. a nucleic acid sequence having a nucleotide sequence of any one of SEQ ID NO: 1, 2 or SEQ ID NO: 28,
b. a nucleic acid sequence having at least 80% identity to the nucleic acid sequence of any one of SEQ ID NO: 1, 2 or SEQ ID NO: 28; or
c. a nucleic acid having a complementary sequence to any one of the nucleic acid of a) or b).

8. The recombinant gene of claim 6 or 7, wherein said plant expressible promoter is selected from the group consisting of constitutive promoter, inducible promoter, tissue specific promoter.

9. The recombinant gene of any one of claims 6 to 8, wherein said plant expressible promoter is a CaMV35S promoter or a Ubiquitin promoter.

10. A vector comprising the recombinant gene of any one of claims 6 to 9.

11. A host cell comprising the recombinant gene of any one of claims 6 to 9 or the vector of claim 10.

12. The host cell of claim 11, which is a bacteria or a wheat plant cell.

13. A wheat plant, plant part or seed consisting of the plant cells according to claim 12.

14. A method for producing a wheat plant with altered number of spikelets per spike comprising the step of altering the abundance of the protein according to claim 1 or 2 within said wheat plant.

15. The method according to claim 14, wherein the abundance of said protein is increased and the number of spikelets per spike is increased compared to the number of spikelets per spike of said wheat plant where the abundance of said protein is not altered.

16. The method according to claim 14, wherein the abundance of said protein is decreased and the number of spikelets per spike is decreased compared to the number of spikelets per spike of said wheat plant where the abundance of said protein is not altered.

17. The method according to claim 14 or 15, wherein the abundance of said protein is increased by providing said wheat plant with:
a. the recombinant gene according to any one of claims 6 to 9, or
b. a heterologous gene encoding the protein according to claim 1 or 2, wherein said heterologous gene is higher expressed than the corresponding endogenous gene.

18. The method according to claim 17, wherein said heterologous gene comprises about 500 bp upstream of the translation start, a nucleotide sequence having the nucleotides from position 4399 to position 4513 of SEQ ID NO: 5, or of a nucleotide sequence having at least 90% sequence identity thereto.

19. The method according to claim 14 or 16, wherein the abundance of said protein is decreased by providing said wheat plant with:
a. a heterologous gene encoding the protein according to claim 1 or 2, wherein said heterologous gene is lower expressed than the endogenous gene, or
b. a mutant allele of the endogenous gene encoding the protein according to claim 1 or 2.

20. The method according to claim 19, wherein said heterologous gene is lower expressed due to the absence of the nucleotide sequence from nucleotide position 4399 to nucleotide position 4513 of SEQ ID NO: 5.

21. The method according to claim 19, wherein said mutant allele is a knock out allele.

22. The method according to claims 17 to 21, wherein the step of providing comprises providing by transformation, crossing, backcrossing, introgressing, targeted genome editing or mutagenesis.

23. A wheat product produced from the seed of claim 13, wherein said wheat product comprises or is meal, ground seeds, flour, or flakes.

24. The wheat product of claim 23, wherein said wheat product comprises an artificial nucleic acid that produces an amplicon diagnostic or specific for the nucleotide sequence of any one of SEQ ID NO: 1, 2, 6, 7, or 28 or a sequence at least 80% identical to any of those sequences.

25. A method of producing the wheat product of claim 23, comprising obtaining seeds comprising an artificial nucleic acid derived from the nucleotide sequence of any one of SEQ ID NO: 1, 2, 6, 7, or 28 or a sequence at least 80% identical to any one of those sequences, and producing said wheat product therefrom.

26. A method of producing wheat flour, wholemeal, starch, starch granules or bran, the method comprising obtaining seed of claim 13 comprising an artificial Apo1 nucleic acid and processing the seed to produce the flour, wholemeal, starch, starch granules or bran.

27. Wheat flour, wholemeal, starch, starch granules or bran produced by the method of claim 26, or comprising an artificial nucleic acid derived from the nucleotide sequence of any one of SEQ ID NO: 1, 2, 6, 7, or 28 or a sequence at least 80% identical to any one of those sequences.

28. A method of producing a food product, comprising mixing the seed of claim 13 or the wheat flour, wholemeal, starch, starch granules or bran from claim 27 with at least one other food ingredient to produce the food product.

29. A method for identifying and/or selecting a wheat plant comprising an allele of a gene contributing positively to the number of spikelets per spike, comprising the step of identifying the presence in the genome of said wheat plant of a nucleic acid having the nucleotide sequence of SEQ ID NO: 5 from nucleotide position 4399 to nucleotide position 4513, or a nucleotide sequence having at least 90% sequence identity thereto.

30. A method for identifying and/or selecting a wheat plant comprising an allele of a gene contributing negatively to the number of spikelets per spike, comprising the step of identifying the absence in the genome of said wheat plant of a nucleic acid having the nucleotide sequence of SEQ ID NO: 5 from nucleotide position 4399 to nucleotide position 4513.
